(19) United States
(12) Patent Application Publication
(10) Pub. No.: US 2024/0376189 A1
(43) Pub. Date: Nov. 14, 2024

(54) HUMANIZED ANTI-C5A ANTIBODIES AND USES THEREOF

(71) Applicants: The Trustees of the University of Pennsylvania, Philadelphia, PA (US); Kira Pharmaceuticals (Suzhou) Ltd., Suzhou, Jiangsu (CN)

(72) Inventors: Ping Tsui, North Potomac, MA (US); Jianjun Zhang, Suzhou (CN); Xihua Zhu, Suzhou (CN); Shigang Qi, Suzhou (CN); Wenchao Song, Bryn Mawr, PA (US); TaKashi Miwa, Bala Cynwyd, PA (US); Sayaka Sato, Philadelphia, PA (US); Damodara Rao Gullipalli, Philadelphia, PA (US)

(21) Appl. No.: 18/696,768

(22) PCT Filed: Sep. 28, 2022
(86) PCT No.: PCT/CN2022/122283

§ 371 Date: Mar. 28, 2024

(30) Foreign Application Priority Data
Sep. 29, 2021 (WO) PCT/CN2021/121959

(51) Int. Cl. C07K 16/18; A61K 39/00; A61P 37/06

(52) U.S. Cl. CPC C07K 16/18; A61P 37/06; A61K 2039/505; C07K 2317/14; C07K 2317/24; C07K 2317/33; C07K 2317/52; C07K 2317/565; C07K 2317/92; C07K 2317/94

(57) ABSTRACT

This invention relates to inhibition of the complement signaling using a humanized anti-C5a antibody. Specifically, the invention relates to methods of treating a complement-mediated disease or complement-mediated disorder in an individual by contacting the individual with the humanized anti-C5a antibody.

### CROSS REFERENCE TO RELATED APPLICATIONS

This application claims priority to and the benefit of International Patent Application PCT/CN2021/121959, filed Sep. 29, 2021, the disclosure of which is hereby incorporated by reference herein in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 792252000941SEQLIST.TXT, date recorded: Sep. 26, 2022, size: 88,835 bytes).

### FIELD OF THE INVENTION

This invention relates to humanized anti-C5a antibody and uses thereof.

### BACKGROUND OF THE INVENTION

The complement system is part of innate immunity that plays a key role in host defense. However, activated complement also has the potential to cause significant tissue injury and destruction and dysregulated complement activity has been found to be associated with a number of rare and common diseases such as paroxysmal nocturnal hemoglobinuria (PNH), atypical hemolytic uremic syndrome, rheumatoid arthritis, age-related macular degeneration, etc. Thus, anti-complement therapy is a promising way of treating these human disorders.

Complement C5 is a critical protein in the terminal pathway of complement activation and is the precursor protein for generating the potent pro-inflammatory mediator C5a, as well as the cytolytic membrane attack complex (MAC), C5b-9.

In some complement-mediated diseases, both C5a and MAC-mediated processes may contribute to pathogenesis, while in other diseases only C5a-mediated inflammation or MAC-mediated cellular injury may be involved. Since complement mediators, including C5a and MAC, also play an important role in host defense against pathogen infection, it is desirable that in therapeutic drug development, we develop anti-complement drugs that are selective, i.e. drugs that will block only the detrimental effect of complement in tissue injury while leaving its normal host defense function intact.

The hemolytic disease PNH is caused by MAC. Other anti-C5 mAbs for the treatment of PNH exist. However, those antibodies unnecessarily block C5a production, putting patients at a greater risk for infection than a therapeutic drug that blocks MAC alone. Likewise, there are complement-mediated diseases that may be mediated primarily by C5a-dependent inflammation (e.g., sepsis) and for such conditions, an anti-C5 mAb drug, while expected to be effective, would unnecessarily block MAC as a side effect.

Thus, there is a need in the art for anti-human C5a mAbs that can inhibit C5a-mediate activity but does not block MAC activity. The present invention addresses and meets these and other needs.

All references cited herein, including patent applications, patent publications, and Genbank Accession numbers are herein incorporated by reference, as if each individual reference were specifically and individually indicated to be incorporated by reference in its entirety.

### BRIEF SUMMARY OF THE INVENTION

The present application provides humanized anti-C5a antibodies, including humanized anti-C5a antibodies having pH-dependent binding to C5a and C5 (the precursor of C5a to which the antibodies also bind but do not functionally block).

In some embodiments, there is provided an isolated humanized antibody that specifically binds to human C5a and C5, wherein the antibody comprises a heavy chain variable domain (VH) and a light chain variable domain (VL), wherein the VH comprises I48M, D54E, and N56W mutations, wherein the VL comprises D28E and D30F mutations, wherein the VH mutation is in reference to SEQ ID NO:1 under the Kabat numbering system, and wherein the VL mutation is in reference to SEQ ID NO:2 under the Kabat numbering system. In some embodiments, the antibody comprises i) a heavy chain CDR1 (“H-CDR1”) comprising the amino acid sequence of SEQ ID NO:3 or a variant thereof comprising one, two, or three amino acid substitutions; ii) a heavy chain CDR2 (“H-CDR2”) comprising the amino acid sequence of SEQ ID NO:4 or a variant thereof comprising one, two, or three amino acid substitutions; iii) a heavy chain CDR3 (“H-CDR3”) comprising the amino acid sequence of SEQ ID NO:5 or a variant thereof comprising one, two, or three amino acid substitutions; iv) a light chain CDR1 (“L-CDR1”) comprising the amino acid sequence of SEQ ID NO:6 or a variant thereof comprising one, two, or three amino acid substitutions; v) a light chain CDR2 (“L-CDR2”) comprising the amino acid sequence of SEQ ID NO:7 or a variant thereof comprising one, two, or three amino acid substitutions; and vi) a light chain CDR3 (L-CDR3”) comprising the amino acid sequence of SEQ ID NO:8 or a variant thereof comprising one, two, or three or amino acid substitutions.

In some embodiments, the antibody further comprises an F29H mutation in VH and a Y96H mutation in the VL, wherein the VH mutation is in reference to SEQ ID NO:1 under the Kabat numbering system, and wherein the VL mutation is in reference to SEQ ID NO:2 under the Kabat numbering system.

In some embodiments, the antibody further comprises an additional substitution in the VH or VL. In some embodiments, the mutation is selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system.

In some embodiments, the antibody comprises: i) a VH comprising the amino acid sequence SEQ ID NO:9 or a variant thereof that is at least about 85%, such as at least about any of 90%, 95%, 96%, 97%, 98%, or 99% identical to SEQ ID NO:9; and ii) a VL comprising the amino acid sequence of SEQ ID NO:10 or a variant thereof that is at least about 85%, such as at least about any of 90%, 95%, 96%, 97%, 98%, or 99% identical to SEQ ID NO:10.

In some embodiments, the antibody comprises: i) H-CDR1 comprising the amino acid sequence of SEQ ID NO:11; ii) H-CDR2 comprising the amino acid sequence of SEQ ID NO:12; iii) H-CDR3 comprising the amino acid sequence of SEQ ID NO:13; iv) L-CDR1 comprising the amino acid sequence of SEQ ID NO:14; v) L-CDR2 comprising the amino acid sequence of SEQ ID NO:15; and vi) L-CDR3 comprising the amino acid sequence of SEQ ID NO:16.

In some embodiments, the antibody comprises: i) a VH comprising the amino acid sequence SEQ ID NO:17; and ii) a VL comprising the amino acid sequence of SEQ ID NO:18.

In some embodiments, the antibody comprises: i) H-CDR1 comprising the amino acid sequence of SEQ ID NO:19; ii) H-CDR2 comprising the amino acid sequence of SEQ ID NO:20; iii) H-CDR3 comprising the amino acid sequence of SEQ ID NO:21; iv) L-CDR1 comprising the amino acid sequence of SEQ ID NO:22; v) L-CDR2 comprising the amino acid sequence of SEQ ID NO:23; and vi) L-CDR3 comprising the amino acid sequence of SEQ ID NO:24.

In some embodiments, the antibody comprises: i) a VH comprising the amino acid sequence SEQ ID NO:25; and ii) a VL comprising the amino acid sequence of SEQ ID NO:26.

In some embodiments, the antibody comprises: i) H-CDR1 comprising the amino acid sequence of SEQ ID NO:27; ii) H-CDR2 comprising the amino acid sequence of SEQ ID NO:28; iii) H-CDR3 comprising the amino acid sequence of SEQ ID NO:29; iv) L-CDR1 comprising the amino acid sequence of SEQ ID NO:30; v) L-CDR2 comprising the amino acid sequence of SEQ ID NO:31; and vi) L-CDR3 comprising the amino acid sequence of SEQ ID NO:32.

In some embodiments, the antibody comprises: i) a VH comprising the amino acid sequence SEQ ID NO:33; and ii) a VL comprising the amino acid sequence of SEQ ID NO:34.

In some embodiments according to any one of the antibodies described herein, the antibody is selected from the group consisting of: a full length antibody, Fab, Fab′, F(ab)2, F(ab′)2, and scFv.

In some embodiments, the antibody further comprises an Fc region. In some embodiments, the Fc region comprises an IgG4 sequence. In some embodiments, the Fc region comprises the amino acid sequence of SEQ ID NO:43 or a variant thereof. In some embodiments, the Fc region comprises one or more mutations selected from the group consisting of S228P, M428L and N434A, wherein the mutations are relative to SEQ ID NO:43 under the EU numbering system. In some embodiments, the Fc region comprises mutations S228P, M428L and N434A. In some embodiments, the Fc region comprises amino acid sequence of SEQ ID NO:44.

In some embodiments according to any one of the antibodies described herein, the low-pH dissociation factor of the antibody dissociating from C5 is between about 40% to about 70%. In some embodiments, the neutral-pH dissociation factor of the antibody dissociating from C5 is between about 0% to about 10%. In some embodiments, the ratio of low-pH dissociation to neutral-pH dissociation is 6 or more.

In some embodiments, the antibody inhibits binding between human C5a to C5aR.

In some embodiments, the antibody has a serum half-life in humans that is at least about 25 days.

In some embodiments, the antibody is manufactured in CHO cells.

In some embodiments, there is provided a nucleic acid encoding any one of the antibodies described herein, comprising the sequence of any one of SEQ ID Nos: 46-53.

In some embodiments, there is provided a vector comprising the nucleic acid of any one of SEQ ID Nos: 46-53.

In some embodiments, there is provided a method for producing any one of the antibodies described herein under a condition sufficient to allow expression of the antibody by cell.

In some embodiments, there is provided a pharmaceutical composition comprising any one of the antibodies described herein and a pharmaceutically acceptable carrier.

In some embodiments there is provided a method for treating an individual having a complement-associated disease or condition comprising administering to the individual an effective amount off the pharmaceutical composition described herein. In some embodiments, the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD), bullous pemphigoid, hidradenitis suppurativa, dermatitis herpetiformis, sweets syndrome, pyoderma gangrenosum, palmo-plantar pustulosis & pustular psoriasis, rheumatoid neutrophilic dermatoses, subcorneal pustular dermatosis, bowel-associated dermatosis-arthritis syndrome, neutrophilic eccrine hidradenitis, linear IgA disease, or any combinations thereof.

In some embodiments, there is provided a method for reducing the activity of a complement system in an individual, comprising administering to the individual an effective amount of the pharmaceutical composition described herein.

In some embodiments according to any one of the antibodies described herein, the antibody cross-reacts with a cyno-monkey C5a or C5.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIGS. 1A-1B shows the results of affinity-ranking ELISA with combination mutant scFv of the anti-C5a constructs using cynomolgus antigen.

FIGS. 2A-2B shows the results of affinity-ranking ELISA with combination mutant scFv of the anti-C5a constructs using human antigen.

FIG. 3 shows the results of C5a binding characterization of anti-C5a antibodies. 4 humanized anti-C5a constructs (16D10, E54, N92 and N97) were serial diluted and added to C5a pre-coated plate, OD450 was measured by microplate reader at 450 nm. Each data point was the mean of 2 replicates.

FIG. 4 shows the results of in vitro activity of anti-C5a antibodies in sheep RBC lysis assay. Antibody-sensitized sheep RBC were incubated with 5% normal human serum in the presence of different concentrations of anti-C5a antibodies. OD405 was measured by microplate reader at 405 nm.

FIG. 5 shows the in vitro activity of anti-C5a antibodies in ligand blocking assay. High control: fluorescence signal of biotinylated C5a binding without anti-C5a antibody; low control: fluorescence signal of cells alone without biotinylated C5a.

FIG. 6 shows the in vitro activity of the anti-C5a antibodies in human C5a induced migration assay using C5aR-expressing U937 cells.

FIG. 7 shows the in vitro activity of the anti-C5a antibodies in C5a-dependent intracellular calcium mobilization assay on a fluorescent imaging plate reader (FLIPR).

FIG. 8 shows the human C5 binding characterization of anti-C5a antibodies.

FIG. 9 depicts a Gator tracing of human C5 association and dissociation of the recombinant E54, N97, N92, and WT(16D10) at pH 5.8 and pH 7.4.

FIG. 10 depicts the % of C5 dissociated from peak value of the recombinant E54, N97, N92, and WT(16D10) at pH 5.8 and pH 7.4.

FIG. 11 shows the pharmacokinetic profiles of anti-C5a antibodies in C5 and FcRn humanized SCID mice.

FIG. 12 shows the Western blot of human C5 protein in in C5/FcRn-humanized SCID mice serum at various time points after singe I.V. dosing of the anti-C5a antibodies.

FIG. 13 shows the result of sandwich ELISA to detect human C5 protein in C5/FcRn-humanized SCID mice serum after single I.V. injection of the anti-C5a antibodies at 25 mg/kg.

FIG. 14 shows the results of C5 titration in sheep RBC lysis assay using C5 depleted normal human serum.

FIG. 15 shows the results of C5 titration in rabbit RBC lysis assay using C5 depleted normal human serum.

FIG. 16 shows a single dose of N92H at 5, 10, 30 mg/kg that was administered intravenously in cynomolgus monkeys (1 male and 1 female per dose group). Concentration was determined using an ELISA method.

FIG. 17 shows a single dose of N92H at 5 mg/kg that was administered intravenously in cynomolgus monkeys (1 male and 1 female). CD11b expression was measured ex-vivo by stimulating whole blood samples with 10 and 30 nM C5a.

FIG. 18 shows the intravenous administration of C5a (10 μg/kg) at 6 hours (hr), 2, 7, and 14 days after N92H infusion. Blood samples were collected 1 minute before and 1 minute after C5a injection for neutrophils counting.

FIG. 19 shows the percent change of neutrophils number in the blood collected at 1 min after C5a injection, relative to the sample collected at 1 min prior to C5a injection, is shown. Each bar represents the mean±standard error from 2 individual monkeys. Cynomolgus monkeys pre-dosed with N92H showed complete inhibition of C5a-induced neutropenia, and this rescue effect can last at least for 14 days.

### DETAILED DESCRIPTION OF THE INVENTION

This invention relates to the inhibition of complement signaling using a humanized anti-C5a antibody. In one embodiment, the invention is directed to inhibiting the complement signaling cascade by specifically inhibiting the function of C5a protein, while leaving C5-b mediated complement pathways minimally disturbed. In one embodiment, the invention is directed to methods of treating and preventing inflammation and autoimmune diseases and disorders mediated by unwanted, uncontrolled, or excessive complement activation. In one embodiment, the invention is directed towards the treatment of a complement-mediated disease or a complement-mediated disorder in an individual by contacting the individual with a humanized anti-C5a antibody.

In various embodiments, the invention is directed to compositions and methods for treating a complement-mediated disease or complement-mediated disorder in an individual by contacting the individual with an anti-C5a antibody. The complement-mediated diseases and disorders that can be treated with the compositions and methods of the invention include, but are not limited to, macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD) or any combinations thereof.

The present application provides novel humanized anti-human C5a antibodies that specifically binds to and inhibit the function of human C5a and comprises a heavy chain variable domain (VH) and a light chain variable domain (VL). In some aspects, the anti-C5a antibodies contain mutations in its VH and/or VL domains. In one aspect, the mutations in the VH and the VL render the antibodies pH sensitive in antigen binding.

In another aspect, there are provided methods of inhibiting complement activation and/or methods of treating diseases (such as complement associated diseases) by administering any one or more of the anti-C5a antibodies or constructs thereof.

In another aspect, there are provided exemplary nucleic acids encoding any one or more of the anti-C5a antibodies or constructs thereof, as well as vectors or host cells comprising such nucleic acids. Methods of making the anti-C5a antibodies are also described.

### I. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “effective amount” and “pharmaceutically effective amount” as used herein refer to a sufficient amount of an agent to provide the desired biological result. That result can be reduction and/or alleviation of the signs, symptoms, or causes of a disease or disorder, or any other desired alteration of a biological system.

As used herein, the terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, mice and humans. In some embodiments, the individual is a human.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody” may refer an immunoglobulin molecule or a fragment thereof which is able to specifically bind to a specific epitope of an antigen (including the basic 4-chain antibody unit). Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), antigen-binding fragments (such as Fv, Fab, Fab′, F(ab)2 and F(ab′)2), as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the VH and V_{L} antibody domains connected into a single polypeptide chain. In some embodiments, the scFv polypeptide further comprises a polypeptide linker between the VH and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (C_{H}) for each of the α and γ chains and four C_{H} domains for and F isotypes. Each L chain has at the N-terminus, a variable domain (V_{L}) followed by a constant domain at its other end. The V_{L} is aligned with the VH and the CL is aligned with the first constant domain of the heavy chain (C_{H}1). Particular amino acid residues are believed to form an interface between the light chain and heavy chain variable domains. The pairing of a VH and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

An “isolated” antibody is one that has been identified, separated and/or recovered from a component of its production environment (E.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelidae species have a single heavy chain variable region, which is referred to as “VHH”. VHH is thus a special type of VH.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present application may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody,” “intact antibody” or “whole antibody” are used interchangeably to refer to an antibody in its substantially intact form, as opposed to an antibody fragment. Specifically full-length 4-chain antibodies include those with heavy and light chains including an Fc region. The constant domains may be native sequence constant domains (e.g., human native sequence constant domains) or amino acid sequence variants thereof. In some cases, the intact antibody may have one or more effector functions.

The term “diabodies” refers to small antibody fragments prepared by constructing sFv fragments (see preceding paragraph) with short linkers (about 5-10) residues) between the VH and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the VH and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATTZFD® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. A suitable human acceptor antibody may be one selected from a conventional database, e.g., the KABAT database, Los Alamos database, the AbM, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see, for example, EP-A-0239400 and EP-A-054951). For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “attach” or “attached” as used herein, refers to connecting or uniting by a bond, link, force or tie in order to keep two or more components together, which encompasses either direct or indirect attachment such that, for example, where a first polypeptide is directly bound to a second polypeptide or material, and, for example, where one or more intermediate compounds (e.g., amino acids, peptides, polypeptides, etc.) are disposed between the first polypeptide and the second polypeptide or material.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, an “immunoassay” refers to any binding assay that uses an antibody capable of binding specifically to a target molecule to detect and quantify the target molecule.

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991)

As use herein, the term “specifically binds” or is “specific for” refers to measurable and reproducible interactions such as binding between a target and an antibody, which is determinative of the presence of the target in the presence of a heterogeneous population of molecules including biological molecules. For example, an antibody that specifically binds a target (which can be an epitope) is an antibody that binds this target with greater affinity, avidity, more readily, and/or with greater duration than it binds other targets. In certain embodiments, an antibody that specifically binds a target has a dissociation constant (Kd) of ≤1 μM, ≤100 nM, ≤10 nM, ≤1 nM, or ≤0.1 nM. In certain embodiments, an antibody specifically binds an epitope on a protein that is conserved among the protein from different species. In another embodiment, specific binding can include, but does not require exclusive binding.

The term “specificity” refers to selective recognition of an antigen binding protein or antibody for a particular epitope of an antigen. Natural antibodies, for example, are monospecific. The term “multispecific” as used herein denotes that an antigen binding protein or an antibody has two or more antigen-binding sites of which at least two bind a different antigen or a different epitope of the same antigen. “Bispecific” as used herein denotes that an antigen binding protein or an antibody has two different antigen-binding specificities. The term “monospecific” antibody as used herein denotes an antibody that has one or more binding sites each of which bind the same epitope of the same antigen.

“Effector cells” are leukocytes which express one or more FcRs and perform effector functions. In one aspect, the effector cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood. Effector cells generally are lymphocytes associated with the effector phase, and function to produce cytokines (helper T cells), killing cells in infected with pathogens (cytotoxic T cells) or secreting antibodies (differentiated B cells).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

An “on-rate,” “rate of association,” “association rate,” or “kₒₙ” as used herein can also be determined as described above using methods such as biolayer interferometry and surface plasmon resonance.

A “low-pH dissociation factor” as used herein is defined as the percentage of antibody dissociated at pH 5.8 from the antigen at 25° C., wherein the antibody is pre-bound to the antigen at pH 7.4. The low-pH dissociation factor may be measured by associating an antibody and an antigen (e.g. the humanized anti-C5a antibody and human C5) at pH 7.4 for 600 seconds, followed by a dissociation period of 600 seconds in a buffer at pH 5.8, and calculation of the percentage of antibody dissociated at pH 5.8 from the antigen. A “neutral-pH dissociation factor” is defined as the percentage of antibody dissociated at pH 7.4 from the antigen at 25° C., wherein the antibody is pre-bound to the antigen at pH 7.4. The neutral-pH dissociation factor may be measured by associating antibody and antigen (e.g. the humanized anti-C5a antibody and human C5) at pH 7.4 for 600 seconds, followed by a dissociation period of 600 seconds in a buffer at pH 7.4, and calculation of the percentage of antibody dissociated at pH 7.4 from the antigen. The antibody-antigen association and dissociation may be measured in various ways that are with the skill in the art, for instance, using biolayer interferometry.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “hybridoma,” as used herein refers to a cell resulting from the fusion of a B-lymphocyte and a fusion partner such as a myeloma cell. A hybridoma can be cloned and maintained indefinitely in cell culture and is able to produce monoclonal antibodies. A hybridoma can also be considered to be a hybrid cell.

The terms “nucleic acid molecule”, “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide. An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts which produce the proteins or errors due to PCR amplification.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Reference to “about” a value or parameter herein includes (and describes) variations that are directed to that value or parameter per se. For example, description referring to “about X” includes description of “X”. As used herein, reference to “not” a value or parameter generally means and describes “other than” a value or parameter. For example, the method is not used to treat cancer of type X means the method is used to treat cancer of types other than X. The term “about X-Y” used herein has the same meaning as “about X to about Y.”

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “sterile” formulation is aseptic or essentially free from living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present application can be hypertonic as a result of the addition of salt and/or buffer.

It is understood that embodiments described herein include “consisting” and/or “consisting essentially of” embodiments.

As used herein and in the appended claims, the singular forms “a,” “or,” and “the” include plural referents unless the context clearly dictates otherwise.

### II. Anti-C5a Antibodies

The present application provides novel, humanized anti-human C5a antibodies and constructs. In some embodiments, the antibody has mutations in its VH and/or VL domains. In some embodiments, the mutations render the antibodies pH sensitive in antigen binding. In some embodiments, the anti-C5a antibodies harbor certain mutations that reduce off-target binding to C5 by rendering binding of the antibody to C5 stronger at a neutral pH (e.g., about pH 7.4; such as that found in the blood) than at a more acidic pH (e.g., about pH 5.8; such as that found in the endosome). In some embodiments, the mutations are histidine mutations, for example the F29H mutation in the VH domain and the Y96H mutation in the VL domain.

The humanized anti-C5a antibodies of the present application comprise at least one antigen binding portion comprising a heavy chain variable domain (VH) and a light chain variable domain (VL). Exemplary antigen binding fragments contemplated herein include, but are not limited to, Fab, Fab′, F(ab′)₂, and Fv fragments; diabodies; linear antibodies; single-chain antibody molecules (such as scFv); and multispecific antibodies formed from antibody fragments. Such antigen binding portion can be a full-length conventional antibody consisting of two heavy chains and two light chains, or an antigen binding fragment derived therefrom.

In some embodiments, the humanized anti-C5a antibody comprises 148M, D54E, and N56W mutations in the VH, wherein the mutations are in reference to SEQ ID NO:1 under the Kabat numbering system. In some embodiments, the humanized anti-C5a antibody comprises D28E and D30F mutations in the VL, wherein the mutations are in reference to SEQ ID NO:2 under the Kabat numbering system. In some embodiments, the humanized anti-C5a antibody comprises a VH comprising I48M, D54E, and N56W mutations in reference to SEQ ID NO:1 and a VL comprising D28E and D30F mutations in reference to SEQ ID NO:2, under the Kabat numbering system. In some embodiments, the humanized anti-C5a antibody comprising I48M, D54E, and N56W mutations in the VH has an improved affinity to human C5a more than 10 folds higher than an anti-C5a antibody comprising SEQ ID NO: 1. In some embodiments, the humanized anti-C5a antibody comprising I48M, D54E, and N56W mutations in the VH has cross-species reactivity with cynomolgus monkey C5a.

In some embodiment, the humanized anti-C5a antibody further comprises one or more additional mutations (such as histidine mutations) in the variable domain (VL) of light chain. For example, in some embodiments, the humanized anti-C5a antibody comprises an F29H mutation in the VH and a Y96H mutation in the VL.

In some embodiments, the humanized anti-C5a antibody further comprises an E54H or an N97H mutation in VH, wherein the VH mutation is in reference to SEQ ID NO:1 under the Kabat numbering system: QVQLQQSDAELVKPGASVKISCKVSGYTFTDHIIHWMNQRPEQGLEWIGYIYPRDGNTN YNENFKGKATLTADKSSSTAYMQLNSLTSEDSAVYFCARERNLEYFDYWGQGTTLTVS S (SEQ ID NO:1)

In some embodiment, the humanized anti-C5a antibody further comprises one or more additional mutations (such as histidine mutations) in the variable domain (VL) of light chain. For example, in some embodiments, the humanized anti-C5a antibody comprises a N92H mutation in VL, wherein the VL mutation is in reference to SEQ ID NO:2 under the Kabat numbering system: DIVLTQSPASLAVSLGQRATISCKASQSVDYDGDNYMNWYQQKPGQPPKLLIYAASNL DSGIPARFSGSGSGTDFTLNIHPVEEEDAATYYCQQSNEDPYTFGGGTKLEIK (SEQ ID NO:2)

In some embodiments, the humanized anti-C5a antibody comprises a mutation selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system.

Thus, in some embodiments, the humanized anti-C5a antibody comprises a VH comprising I48M, D54E, and N56W mutations in reference to SEQ ID NO:1 and a VL comprising D28E and D30F mutations in reference to SEQ ID NO:2. In some embodiments, the humanized anti-C5a antibody further comprises an F29H mutation in the VH in reference to SEQ ID NO:1 and a Y96H mutation in the VL in reference to SEQ ID NO:2. In some embodiments, the humanized anti-C5a antibody further comprises a mutation selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system.

In some embodiments, the humanized anti-C5a antibody comprises an Fc region, such as a human Fc region. In some embodiments, the Fc region is derived from an IgG molecule, such as any one of the IgG1, IgG2, IgG3, or IgG4 subclass. In some embodiments, the Fc region is capable of mediating an antibody effector function, such as ADCC (antibody-dependent cell-mediated cytotoxicity) and/or CDC (complement-dependent cytotoxicity). For example, antibodies of subclass IgG1, IgG2, and IgG3 with wild-type Fc sequences usually show complement activation including CIq and C3 binding, whereas IgG4 does not activate the complement system and does not bind CIq and/or C3. In some embodiments, the Fc region comprises a modification that reduces binding affinity of the Fc region to an Fc receptor. In some embodiments, the Fc region is an IgG4 Fc. In some embodiments, the IgG4 Fc region comprises the amino acid sequence of SEQ ID NO: 43. In some embodiments, the IgG4 Fc comprises mutations. See, for example, Armour K L et al., Eur J. Immunol. 1999; 29: 2613; and Shields R L et al., J. Biol. Chem. 2001; 276: 6591. In some embodiments, the Fc region comprises one or more mutations selected from the group consisting of S228P, M428L, and N434A, wherein the mutations are relative to SEQ ID NO:43 under the EU numbering system. In some embodiments, the Fc region comprises mutations S228P, M428L, and N434A. In some embodiments, the IgG4 Fc region comprises the amino acid sequence of SEQ ID NO:44.

In some embodiments, the humanized anti-C5a antibody (for example, any one of the humanized anti-C5a antibodies described herein comprising a VH comprising I48M, D54E, and N56W mutations in reference to SEQ ID NO:1 and a VL comprising D28E and D30F mutations in reference to SEQ ID NO:2) comprises a heavy chain CDR1 (“H-CDR1”) comprising the amino acid sequence of SEQ ID NO:3 or a variant thereof comprising one, two, or three amino acid substitutions; a heavy chain CDR2 (“H-CDR2”) comprising the amino acid sequence of SEQ ID NO:4 or a variant thereof comprising one, two, or three amino acid substitutions; a heavy chain CDR3 (“H-CDR3”) comprising the amino acid sequence of SEQ ID NO:5 or a variant thereof comprising one, two, or three amino acid substitutions; a light chain CDR1 (“L-CDR1”) comprising the amino acid sequence of SEQ ID NO:6 or a variant thereof comprising one, two, or three amino acid substitutions; a light chain CDR2 (“L-CDR2”) comprising the amino acid sequence of SEQ ID NO:7 or a variant thereof comprising one, two, or three amino acid substitutions; and a light chain CDR3 (“L-CDR3”) comprising the amino acid sequence of SEQ ID NO:8 or a variant thereof comprising one, two, or three amino acid substitutions. In some embodiments, the humanized anti-C5a antibody comprises a heavy chain CDR1 (“H-CDR1”) comprising the amino acid sequence of SEQ ID NO:3; a heavy chain CDR2 (“H-CDR2”) comprising the amino acid sequence of SEQ ID NO:4; a heavy chain CDR3 (“H-CDR3”) comprising the amino acid sequence of SEQ ID NO:5; a light chain CDR1 (“L-CDR1”) comprising the amino acid sequence of SEQ ID NO:6; a light chain CDR2 (“L-CDR2”) comprising the amino acid sequence of SEQ ID NO:7; and a light chain CDR3 (“L-CDR3”) comprising the amino acid sequence of SEQ ID NO: 8.

In some embodiments (independent of or in addition to the CDR sequences described here), the humanized anti-C5a antibody comprises a VH comprising the amino acid sequence SEQ ID NO:9 or a variant thereof that is at least about any one of 60%, 70%, 80%, 85%, 90%, 95%, 96%, 97%, 98%, 99% or more of amino acid homology to SEQ ID NO:9; and a VL comprising the amino acid sequence of SEQ ID NO:10 or a variant thereof that is at least about any one of 60%, 70%, 80%, 85%, 90%, 95%, 96%, 97%, 98%, 99% or more of amino acid homology to SEQ ID NO:10. In some embodiments, the humanized anti-C5a antibody comprises a VH comprising the amino acid sequence SEQ ID NO:9 and a VL comprising the amino acid sequence of SEQ ID NO:10. In some embodiments, the humanized anti-C5a antibody further comprises an Fc region (such as the Fc region of an IgG4).

In some embodiments, the humanized anti-C5a antibody (for example, any one of the humanized anti-C5a antibodies described herein comprising a VH comprising I48M, D54E, and N56W mutations in reference to SEQ ID NO:1 and a VL comprising D28E and D30F mutations in reference to SEQ ID NO:2) comprises a heavy chain CDR1 (“H-CDR1”) comprising the amino acid sequence of SEQ ID NO:11 or a variant thereof comprising one, two, or three amino acid substitutions; a heavy chain CDR2 (“H-CDR2”) comprising the amino acid sequence of SEQ ID NO:12 or a variant thereof comprising one, two, or three amino acid substitutions; a heavy chain CDR3 (“H-CDR3”) comprising the amino acid sequence of SEQ ID NO:13 or a variant thereof comprising one, two, or three amino acid substitutions; a light chain CDR1 (“L-CDR1”) comprising the amino acid sequence of SEQ ID NO:14 or a variant thereof comprising one, two, or three amino acid substitutions; a light chain CDR2 (“L-CDR2”) comprising the amino acid sequence of SEQ ID NO:15 or a variant thereof comprising one, two, or three amino acid substitutions; and a light chain CDR3 (“L-CDR3”) comprising the amino acid sequence of SEQ ID NO:16 or a variant thereof comprising one, two, or three amino acid substitutions. In some embodiments, the humanized anti-C5a antibody comprises a heavy chain CDR1 (“H-CDR1”) comprising the amino acid sequence of SEQ ID NO:11; a heavy chain CDR2 (“H-CDR2”) comprising the amino acid sequence of SEQ ID NO:12; a heavy chain CDR3 (“H-CDR3”) comprising the amino acid sequence of SEQ ID NO:13; a light chain CDR1 (“L-CDR1”) comprising the amino acid sequence of SEQ ID NO:14; a light chain CDR2 (“L-CDR2”) comprising the amino acid sequence of SEQ ID NO:15; and a light chain CDR3 (“L-CDR3”) comprising the amino acid sequence of SEQ ID NO:16.

In some embodiments (independent of or in addition to the CDR sequences described here), the humanized anti-C5a antibody comprises a VH comprising the amino acid sequence SEQ ID NO:17 or a variant thereof that is at least about any one of 60%, 70%, 80%, 85%, 90%, 95%, 96%, 97%, 98%, 99% or more of amino acid homology to SEQ ID NO:17; and a V_{L} comprising the amino acid sequence of SEQ ID NO: 18 or a variant thereof that is at least about any one of 60%, 70%, 80%, 85%, 90%, 95%, 96%, 97%, 98%, 99% or more of amino acid homology to SEQ ID NO:18. In some embodiments, the humanized anti-C5a antibody comprises a VH comprising the amino acid sequence SEQ ID NO:17 and a VL comprising the amino acid sequence of SEQ ID NO:18. In some embodiments, the humanized anti-C5a antibody further comprises an Fc region (such as the Fc region of an IgG4).

In some embodiments, the humanized anti-C5a antibody (for example, any one of the humanized anti-C5a antibodies described herein comprising a VH comprising I48M, D54E, and N56W mutations in reference to SEQ ID NO:1 and a VL comprising D28E and D30F mutations in reference to SEQ ID NO:2) comprises a heavy chain CDR1 (“H-CDR1”) comprising the amino acid sequence of SEQ ID NO:19 or a variant thereof comprising one, two, or three amino acid substitutions; a heavy chain CDR2 (“H-CDR2”) comprising the amino acid sequence of SEQ ID NO:20 or a variant thereof comprising one, two, or three amino acid substitutions; a heavy chain CDR3 (“H-CDR3”) comprising the amino acid sequence of SEQ ID NO:21 or a variant thereof comprising one, two, or three amino acid substitutions; a light chain CDR1 (“L-CDR1”) comprising the amino acid sequence of SEQ ID NO:22 or a variant thereof comprising one, two, or three amino acid substitutions; a light chain CDR2 (“L-CDR2”) comprising the amino acid sequence of SEQ ID NO:23 or a variant thereof comprising one, two, or three amino acid substitutions; and a light chain CDR3 (“L-CDR3”) comprising the amino acid sequence of SEQ ID NO:24 or a variant thereof comprising one, two, or three amino acid substitutions. In some embodiments, the humanized anti-C5a antibody comprises a heavy chain CDR1 (“H-CDR1”) comprising the amino acid sequence of SEQ ID NO:19; a heavy chain CDR2 (“H-CDR2”) comprising the amino acid sequence of SEQ ID NO:20; a heavy chain CDR3 (“H-CDR3”) comprising the amino acid sequence of SEQ ID NO:21; a light chain CDR1 (“L-CDR1”) comprising the amino acid sequence of SEQ ID NO:22; a light chain CDR2 (“L-CDR2”) comprising the amino acid sequence of SEQ ID NO:23; and a light chain CDR3 (“L-CDR3”) comprising the amino acid sequence of SEQ ID NO:24.

In some embodiments (independent of or in addition to the CDR sequences described here), the humanized anti-C5a antibody comprises a VH comprising the amino acid sequence SEQ ID NO:25 or a variant thereof that is at least about any one of 60%, 70%, 80%, 85%, 90%, 95%, 96%, 97%, 98%, 99% or more of amino acid homology to SEQ ID NO:25; and a VL comprising the amino acid sequence of SEQ ID NO:26 or a variant thereof that is at least about any one of 60%, 70%, 80%, 85%, 90%, 95%, 96%, 97%, 98%, 99% or more of amino acid homology to SEQ ID NO:26. In some embodiments, the humanized anti-C5a antibody comprises a VH comprising the amino acid sequence SEQ ID NO:25 and a VL comprising the amino acid sequence of SEQ ID NO:26. In some embodiments, the humanized anti-C5a antibody further comprises an Fc region (such as the Fc region of an IgG4).

In some embodiments, the humanized anti-C5a antibody (for example, any one of the humanized anti-C5a antibodies described herein comprising a VH comprising I48M, D54E, and N56W mutations in reference to SEQ ID NO:1 and a VL comprising D28E and D30F mutations in reference to SEQ ID NO:2) comprises a heavy chain CDR1 (“H-CDR1”) comprising the amino acid sequence of SEQ ID NO:27 or a variant thereof comprising one, two, or three amino acid substitutions; a heavy chain CDR2 (“H-CDR2”) comprising the amino acid sequence of SEQ ID NO:28 or a variant thereof comprising one, two, or three amino acid substitutions; a heavy chain CDR3 (“H-CDR3”) comprising the amino acid sequence of SEQ ID NO:29 or a variant thereof comprising one, two, or three amino acid substitutions; a light chain CDR1 (“L-CDR1”) comprising the amino acid sequence of SEQ ID NO:30 or a variant thereof comprising one, two, or three amino acid substitutions; a light chain CDR2 (“L-CDR2”) comprising the amino acid sequence of SEQ ID NO:31 or a variant thereof comprising one, two, or three amino acid substitutions; and a light chain CDR3 (“L-CDR3”) comprising the amino acid sequence of SEQ ID NO:32 or a variant thereof comprising one, two, or three amino acid substitutions. In some embodiments, the humanized anti-C5a antibody comprises a heavy chain CDR1 (“H-CDR1”) comprising the amino acid sequence of SEQ ID NO:27; a heavy chain CDR2 (“H-CDR2”) comprising the amino acid sequence of SEQ ID NO:28; a heavy chain CDR3 (“H-CDR3”) comprising the amino acid sequence of SEQ ID NO:29; a light chain CDR1 (“L-CDR1”) comprising the amino acid sequence of SEQ ID NO:30; a light chain CDR2 (“L-CDR2”) comprising the amino acid sequence of SEQ ID NO:31; and a light chain CDR3 (“L-CDR3”) comprising the amino acid sequence of SEQ ID NO:32.

In some embodiments (independent of or in addition to the CDR sequences described here), the humanized anti-C5a antibody comprises a VH comprising the amino acid sequence SEQ ID NO:33 or a variant thereof that is at least about any one of 60%, 70%, 80%, 85%, 90%, 95%, 96%, 97%, 98%, 99% or more of amino acid homology to SEQ ID NO:33; and a VL comprising the amino acid sequence of SEQ ID NO:34 or a variant thereof that is at least about any one of 60%, 70%, 80%, 85%, 90%, 95%, 96%, 97%, 98%, 99% or more of amino acid homology to SEQ ID NO:34. In some embodiments, the humanized anti-C5a antibody comprises a VH comprising the amino acid sequence SEQ ID NO:33 and a VL comprising the amino acid sequence of SEQ ID NO:34. In some embodiments, the humanized anti-C5a antibody further comprises an Fc region (such as the Fc region of an IgG4).

In some embodiments, the humanized anti-C5a antibody comprises H-CDR1, H-CDR2, and H-CDR3 sequences of an antibody having a VH domain of SEQ ID NO: 54 and L-CDR1, L-CDR2, and L-CDR3 sequences of an antibody having a VL domain of SEQ ID NO: 55.

In some embodiments, the humanized anti-C5a antibody comprises H-CDR1, H-CDR2, and H-CDR3 sequences of an antibody having a VH domain of SEQ ID NO: 56 and L-CDR1, L-CDR2, and L-CDR3 sequences of an antibody having a VL domain of SEQ ID NO: 57.

In some embodiments, the humanized anti-C5a antibody comprises H-CDR1, H-CDR2, and H-CDR3 sequences of an antibody having a VH domain of SEQ ID NO: 58 and L-CDR1, L-CDR2, and L-CDR3 sequences of an antibody having a VL domain of SEQ ID NO: 59.

In some embodiments, the humanized anti-C5a antibody comprises H-CDR1, H-CDR2, and H-CDR3 sequences of an antibody having a VH domain of SEQ ID NO: 60 and L-CDR1, L-CDR2, and L-CDR3 sequences of an antibody having a VL domain of SEQ ID NO: 61.

In some embodiments, the humanized anti-C5a antibody comprises H-CDR1, H-CDR2, and H-CDR3 sequences of an antibody having a VH domain of SEQ ID NO: 62 and L-CDR1, L-CDR2, and L-CDR3 sequences of an antibody having a VL domain of SEQ ID NO: 63.

In some embodiments, the humanized anti-C5a antibody comprises H-CDR1, H-CDR2, and H-CDR3 sequences of an antibody having a VH domain of SEQ ID NO: 64 and L-CDR1, L-CDR2, and L-CDR3 sequences of an antibody having a VL domain of SEQ ID NO: 65.

In some embodiments, the humanized anti-C5a antibody comprises H-CDR1, H-CDR2, and H-CDR3 sequences of an antibody having a VH domain of SEQ ID NO: 66 and L-CDR1, L-CDR2, and L-CDR3 sequences of an antibody having a VL domain of SEQ ID NO: 67.

In some embodiments, the humanized anti-C5a antibody comprises H-CDR1, H-CDR2, and H-CDR3 sequences of an antibody having a VH domain of SEQ ID NO: 68 and L-CDR1, L-CDR2, and L-CDR3 sequences of an antibody having a VL domain of SEQ ID NO: 69.

In some embodiments, the humanized anti-C5a antibody comprises H-CDR1, H-CDR2, and H-CDR3 sequences of an antibody having a VH domain of SEQ ID NO: 70 and L-CDR1, L-CDR2, and L-CDR3 sequences of an antibody having a VL domain of SEQ ID NO: 71.

In some embodiments, the humanized anti-C5a antibody comprises H-CDR1, H-CDR2, and H-CDR3 sequences of an antibody having a VH domain of SEQ ID NO: 72 and L-CDR1, L-CDR2, and L-CDR3 sequences of an antibody having a VL domain of SEQ ID NO: 73.

In some embodiments, the humanized anti-C5a antibody comprises H-CDR1, H-CDR2, and H-CDR3 sequences of an antibody having a VH domain of SEQ ID NO: 74 and L-CDR1, L-CDR2, and L-CDR3 sequences of an antibody having a VL domain of SEQ ID NO: 75.

In some embodiments, the humanized anti-C5a antibody comprises H-CDR1, H-CDR2, and H-CDR3 sequences of an antibody having a VH domain of SEQ ID NO: 76 and L-CDR1, L-CDR2, and L-CDR3 sequences of an antibody having a VL domain of SEQ ID NO: 77.

In some embodiments, the humanized anti-C5a antibody comprises H-CDR1, H-CDR2, and H-CDR3 sequences of an antibody having a VH domain of SEQ ID NO: 78 and L-CDR1, L-CDR2, and L-CDR3 sequences of an antibody having a VL domain of SEQ ID NO: 79.

In some embodiments, the humanized anti-C5a antibody comprises H-CDR1, H-CDR2, and H-CDR3 sequences of an antibody having a VH domain of SEQ ID NO: 80 and L-CDR1, L-CDR2, and L-CDR3 sequences of an antibody having a VL domain of SEQ ID NO: 81.

In some embodiments, the humanized anti-C5a antibody comprises H-CDR1, H-CDR2, and H-CDR3 sequences of an antibody having a VH domain of SEQ ID NO: 82 and L-CDR1, L-CDR2, and L-CDR3 sequences of an antibody having a VL domain of SEQ ID NO: 83.

It is to be understood that although a Kabat numbering system is used for referencing mutations, the positions of mutations may be denoted under different numbering systems as shown in Table 1 below using exemplary sequences.

TABLE 1
Amino Acid Substitutions under different numbering systems

| | | | Position |
|---|---|---|---|
| | | | in the |
| | | | VH/VL |
| Amino acid substitution (in bold) | Kabat | IMGT | sequence |
| QVQLVQSGAEVKKPGSSVKVSCKASGYTFTDHIIHWMRQA | I48M | I53M | I48M |
| PGQGLEWMGYIYPREGWTNYNENFKGRVTITADKSTSTAY | | | |
| MELSSLRSEDTAVYYCARERNLEYFDYWGQGTTVTVSS | | | |
| (VH, SEQ ID NO: 9) | | | |
| QVQLVQSGAEVKKPGSSVKVSCKASGYTFTDHIIHWMRQA | D54E | D62E | D55E |
| PGQGLEWMGYIYPREGWTNYNENFKGRVTITADKSTSTAY | | | |
| MELSSLRSEDTAVYYCARERNLEYFDYWGQGTTVTVSS | | | |
| (VH, SEQ ID NO: 9) | | | |
| QVQLVQSGAEVKKPGSSVKVSCKASGYTFTDHIIHWMRQA | N56W | N64W | N57W |
| PGQGLEWMGYIYPREGWTNYNENFKGRVTITADKSTSTAY | | | |
| MELSSLRSEDTAVYYCARERNLEYFDYWGQGTTVTVSS | | | |
| (VH, SEQ ID NO: 9) | | | |
| QVQLVQSGAEVKKPGSSVKVSCKASGYTHTDHIIHWMRQ | F29H | F30H | F29H |
| APGQGLEWMGYIYPRHGWTNYNENFKGRVTITADKSTST | | | |
| AYMELSSLRSEDTAVYYCARERNLEYFDYWGQGTTVTVSS | | | |
| (VH, SEQ ID NO: 17) | | | |
| QVQLVQSGAEVKKPGSSVKVSCKASGYTHTDHIIHWMRQ | E54H | E62H | E55H |
| APGQGLEWMGYIYPRHGWTNYNENFKGRVTITADKSTST | | | |
| AYMELSSLRSEDTAVYYCARERNLEYFDYWGQGTTVTVSS | | | |
| (VH, SEQ ID NO: 17) | | | |
| QVQLVQSGAEVKKPGSSVKVSCKASGYTHTDHIIHWMRQ | N97H | N109H | N101H |
| APGQGLEWMGYIYPREGWTNYNENFKGRVTITADKSTSTA | | | |
| YMELSSLRSEDTAVYYCARERHLEYFDYWGQGTTVTVSS | | | |
| (VH, SEQ ID NO: 33) | | | |
| DIVLTQSPASLAVSPGQRATITCKASQSVDYEGFNYMNWY | D28E | D34E | D32E |
| QQKPGQPPKLLIYAASNLDSGVPARFSGSGSGTDFTLTINPV | | | |
| EAEDTANYYCQQSNEDPYTFGGGTKVEIK | | | |
| (VL, SEQ ID NO: 10) | | | |
| DIVLTQSPASLAVSPGQRATITCKASQSVDYEGFNYMNWY | D30F | D36F | D34F |
| QQKPGQPPKLLIYAASNLDSGVPARFSGSGSGTDFTLTINPV | | | |
| EAEDTANYYCQQSNEDPYTFGGGTKVEIK | | | |
| (VL, SEQ ID NO: 10) | | | |
| DIVLTQSPASLAVSPGQRATITCKASQSVDYEGFNYMNWY | N92H | N108H | N96H |
| QQKPGQPPKLLIYAASNLDSGVPARFSGSGSGTDFTLTINPV | | | |
| EAEDTANYYCQQSHEDPHTFGGGTKVEIK | | | |
| (VL, SEQ ID NO: 26) | | | |
| DIVLTQSPASLAVSPGQRATITCKASQSVDYEGFNYMNWY | Y96H | Y116H | Y100H |
| QQKPGQPPKLLIYAASNLDSGVPARFSGSGSGTDFTLTINPV | | | |
| EAEDTANYYCQQSHEDPHTFGGGTKVEIK | | | |
| (VL, SEQ ID NO: 26) | | | |

### C5a Proteins and C5a Binding Analysis

The complement system plays an important role in the pathology of many autoimmune, inflammatory and ischemic diseases. Inappropriate complement activation and its deposition on host cells can lead to complement-mediated lysis and/or injury of cells and target tissues, as well as tissue destruction due to the generation of powerful mediators of inflammation. The complement system, also known as complement cascade, is a part of the immune system that enhances (complements) the ability of antibodies and phagocytic cells to clear microbes and damaged cells from an organism, promote inflammation, and attack the pathogen's cell membrane. It is part of the innate immune system, which is not adaptable and does not change during an individual's lifetime. The complement system can, however, be recruited and brought into action by antibodies generated by the adaptive immune system.

Without being bound by any theory or hypothesis, there are three known complement pathways: the alternative complement pathway (AP), the classical pathway (CP), and the lectin pathway (LP). Generally, the CP is initiated by antigen-antibody complexes, the LP is activated by binding of lectins to sugar molecules on microbial surfaces, while the AP is constitutively active at a low level but can be quickly amplified on bacterial, viral, and parasitic cell surfaces due to the lack of regulatory proteins. Host cells are usually protected from AP complement activation by regulatory proteins. But in some situations, such as when the regulatory proteins are defective or missing, the AP can also be activated uncontrollably on host cells, leading to complement-mediated disease or disorder. The CP consists of components C1, C2, C4 and converges with the AP at the C3 activation step. The LP consists of mannose-binding lectins (MBLs) and MBL-associated serine proteases (MASPs) and shares with the CP the components C4 and C2. The AP consists of components C3 and several factors, such as factor B, factor D, properdin, and the fluid phase regulator factor H. Complement activation consists of three stages: (a) recognition, (b) enzymatic activation, and (c) membrane attack leading to cell death. The first phase of CP complement activation begins with C1. C1 is made up of three distinct proteins: a recognition subunit, C1q, and the serine protease subcomponents, C1r and CIs, which are bound together in a calcium-dependent tetrameric complex, C1r2 s2. An intact C1 complex is necessary for physiological activation of C1 to result. Activation occurs when the intact C1 complex binds to immunoglobulin complexed with antigen. This binding activates C1s which then cleaves both the C4 and C2 proteins to generate C4a and C4b, as well as C2a and C2b. The C4b and C2a fragments combine to form the C3 convertase, C4b2a, which in turn cleaves C3 to form C3a and C3b. Activation of the LP is initiated by MBL binding to certain sugars on the target surface and this triggers the activation of MBL-associated serine proteases (MASPs) which then cleave C4 and C2 in a manner analogous to the activity of CIs of the CP, resulting in the generation of the C3 convertase, C4b2a. Thus, the CP and LP are activated by different mechanisms but they share the same components C4 and C2 and both pathways lead to the generation of the same C3 convertase, C4b2a. The cleavage of C3 by C4b2a into C3b and C3a is a central event of the complement pathway for two reasons. It initiates the AP amplification loop because surface deposited C3b is a central intermediate of the AP C3 convertase C3bBb. Both C3a and C3b are biologically important. C3a is proinflammatory and together with C5a are referred to as anaphylatoxins. C3b and its further cleavage products also bind to complement receptors present on neutrophils, eosinophils, monocytes and macrophages, thereby facilitating phagocytosis and clearance of C3b-opsonized particles. Finally, C3b can associate with C4b2a or C3bBb to form the C5 convertase of the CP and LP, and AP, respectively, to activate the terminal complement sequence, leading to the production of C5a, a potent proinflammatory mediator, and the assembly of the lytic membrane attack complex (MAC), C5-C9.

Defective complement action is a cause of several human glomerular diseases including atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, renal ischemic reperfusion injury, lupus nephritis, membranous nephropathy, and chronic transplant mediated glomerulopathy. Aberrant complement component activation has been proposed as markers in various types of cancers and their clinical outcomes. Lung cancer patients show significantly higher plasma levels of complement proteins and activation fragments than do control donors, and elevated complement levels are correlated with lung tumor size. Complement-related proteins are also elevated in biological fluids from patients with other types of tumor. See, for example, Pio et al. Semin Immunol. 2013 February; 25(1): 54-64. Inhibition of the complement cascade has been proposed for glomerular diseases and cancer treatment.

C5a is a 74 AA anaphylatoxin (SEQ ID NO: 45), generated from cleavage of complement component C5. Mature C5 is cleaved into the C5a and C5b fragments during activation of the complement pathways. C5a is cleaved from the alpha chain of C5 by C5 convertase as an amino terminal fragment comprising the first 74 amino acids of the alpha chain. The remaining portion of mature C5 is fragment C5b, which contains the rest of the alpha chain disulfide bonded to the beta chain. Approximately 20% of the 11 kDa mass of C5a is attributed to carbohydrate. C5a acts as a highly inflammatory peptide, encouraging complement activation, formation of the MAC, attraction of innate immune cells, and histamine release involved in allergic responses. C5a is an anaphylatoxin, causing increased expression of adhesion molecules on endothelium, contraction of smooth muscle, and increased vascular permeability. C5a des-Arg is a much less potent anaphylatoxin. Both C5a and C5a des-Arg can trigger mast cell degranulation, releasing proinflammatory molecules histamine and TNF-α. C5a is also an effective chemoattractant, initiating accumulation of complement and phagocytic cells at sites of infection or recruitment of antigen-presenting cells to lymph nodes. C5a plays a key role in increasing migration and adherence of neutrophils and monocytes to vessel walls. White blood cells are activated by upregulation of integrin avidity, the lipoxygenase pathway and arachidonic acid metabolism. C5a also modulates the balance between activating versus inhibitory IgG Fc receptors on leukocytes, thereby enhancing the autoimmune response. See, for example, Manthey H D, Woodruff™, Taylor S M, Monk P N (November 2009). “Complement component 5a (C5a)”. The International Journal of Biochemistry & Cell Biology. 41 (11): 2114-7.

C5a binds to C5aR (also known as CD88) on the surface of target cells such as macrophages, neutrophils and endothelial cells, and triggers signal transduction to promote inflammatory responses. C5a/C5aR signal favors MDSC proliferation and thus restricting T cell immunity. Inhibition of the C5a/C5aR pathways has been proposed to ameliorate inflammatory diseases and have potentials in immune-oncology.

Binding affinity and specificity of the humanized anti-C5a antibody described herein can be determined experimentally by methods known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), Bio-Layer Interferometry (BLI) (e.g. Octet system, ForteBio), RIA, ECL, IRMA, EIA, peptide scans, and enzyme-linked immunosorbent assay (ELISA). See, e.g., Benny K. C. Lo (2004) “Antibody Engineering: Methods and Protocols.” Humana Press (ISBN: 1588290921); Borrebaek (1992) “Antibody Engineering, A Practical Guide.” W.H. Freeman and Co., N.Y.; Borrebaek (1995) “Antibody Engineering.” 2nd Edition, Oxford University Press, N.Y., Oxford; Johne et al. (1993). J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants by BLI) are set forth in the working examples.

Methods for determining whether a particular antibody described herein inhibits C5a are known in the art. Inhibition of C5a can reduce chemotactic migration of cells, as shown in Schraufstatter et al. J Immunol Mar. 15, 2009, 182 (6) 3827-3836. C5a function may thus be determined by, for example, cell migration assays. See, for example, Rousseau, Simon, et al. Cellular signalling 18.11 (2006): 1897-1905. C5a has been shown to induce calcium influx into cells (Moller et al., J Neurosci. 1997 Jan. 15; 17(2): 615-624). Calcium influx assays can be used to measure C5a activity, for example, by using calcium staining methods as described in Farkas et al., Neuroscience Volume 86, Issue 3, 1998, Pages 903-911, or radiometric techniques (Monk et al., Biochem J (1993) 295 (3): 679-684).

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay in chicken erythrocyte hemolysis method as described in, e.g., Hillmen et al. (2004) N Engl. J Med 350(6):552. Methods for determining whether a candidate compound inhibits the cleavage of human C5 into forms C5a and C5b are known in the art and described in, e.g., Thomas et al. (1996) Mol Immunol 33(17-18): 1389-401; and Evans et al. (1995) Mol Immunol 32(16): 1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Wurzner et al. (1991) Complement Inflamm 8:328-340). For C5b, hemolytic assays or assays for soluble C5b-9 as discussed herein can be used. Other assays known in the art can also be used. Using assays of these or other suitable types, candidate agents capable of inhibiting human complement component C5 can be screened.

Hemolytic assays can be used to determine the inhibitory activity of an anti-C5a antibody on C5-mediated complement activation, and are thus useful for determining potential off-target binding of anti-C5a antibodies. In order to determine the effect of the humanized anti-C5a antibody on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. To determine the effect of the humanized anti-C5a antibody on alternative pathway-mediated hemolysis, unsensitized rabbit or guinea pig erythrocytes are used as the target cells. The percentage of lysis is normalized by considering 100% lysis equal to the lysis occurring in the absence of the inhibitor.

### “TMDD” or “Sink Effect” and pH Dependent Anti-C5a Antibodies

Most of the therapeutic monoclonal antibodies show non-linear dose dependent clearance due to the Target Mediated Drug Disposition or TMDD, also nick named “sink effect”.

For instances, when antibody was administered at a low or a sub-therapeutic dose, it mainly binds to target, very little free antibody. When the dose increases, the percent of free antibody increases over antibody/antigen binding complex. When the dose increases to a very high level, the antigen is fully saturated or bound, and majority of total antibody is unbound or free. Free mAb has a longer half-life (due to the FcRn mechanism) than to the mAb:Ag binding complex which is cleared by phagocytic process. Therefore, at the low doses, the half-life of total antibody (mostly in bound state) is shorter, and the half-life of total antibody increases as dose increases until reaching to a plateau where the total antibody is dominated by unbound or free antibody. Clearance of antibody that binds to membrane antigen follows the same paradigm. At low dose, clearance is faster as the unbound targets will “sop up” antibody, serving as a sink (also referred to as the “antigen sink”, Keizer et al., Clin Pharmacokinet. 2010 August; 49(8):493-507, Eser et al., Curr Opin Gastroenterol. 2013 July; 29(4):391-6).

As C5a is a part of C5, anti-C5a antibodies can bind to C5 well. The high capability C5 binding by anti-C5a antibody and the large excess of serum C5 concentration than C5a make C5 a binding sink to the anti-C5a antibodies, leading to fast clearance in vivo, or the C5 “sink effect” on anti-C5a antibodies.

In some embodiments, the humanized anti-C5a antibodies described herein bind to C5 but does not inhibit C5 cleavage by C5 convertase. In some embodiments, the humanized anti-C5a antibodies bind to and reduce C5 level via an unknown mechanism of action. In some embodiments, the reduction of C5 level concurrently result in reduced C5a level and/or reduced sensitivity of C5 mediated drug disposition. In some embodiments, reduced C5a level and/or reduced sensitivity of C5 mediated drug disposition leading to sustained drug effects.

In some embodiments, the humanized anti-C5a antibodies described herein possess pH-dependent dissociation from C5. Such pH-dependent binding provides for greater persistence of administered antibody or antibody fusion protein molecules, because immune complexes (i.e., the humanized anti-C5a antibody bound to C5) taken up by cells will dissociate in the acidic environment of the endosome and allow the freed antibody or antibody fusion protein to be recycled back out of the cell through the neonatal Fc receptor (FcRn) where it is available to bind to a new C5a molecule.

In some embodiments, the humanized anti-C5a antibody described possesses pH-dependent binding to C5. As used herein, the expression “pH-dependent binding” means that the antibody exhibits reduced binding to C5 at acidic pH (e.g. about pH 5.8; such as in the endosome) as compared to its binding at neutral pH (e.g., about pH 7.4; such as in the blood).

pH-dependency of the humanized anti-C5a antibody described herein can be determined experimentally by methods known in the art, such as in U.S. Pat. No. 9,079,949, and WO2016/098356. pH-dependency may be reflected in the differences in binding properties such as binding affinity (e.g. dissociation constant), kinetic parameters (e.g. association rate and dissociation rate), and percentage dissociation, at different pH levels. In some embodiments, the pH-dependency of the humanized anti-C5a antibody described herein may be expressed in terms of the ratio of the percentage dissociation. In some embodiments, the percentage dissociation may be expressed in terms of the low-pH dissociation factor and the neutral-pH dissociation factor.

The pH dependence of the humanized anti-C5a antibody can be assessed based on the dissociation of a C5-bound antibody at an acidic pH (e.g., pH 5.8) or at a neutral pH (e.g., pH 7.4). Low-pH dissociation factor, namely, the percentage of antibody dissociated at pH 5.8 from the antigen at 25° C., wherein the antibody is pre-bound to the antigen at pH 7.4, can be used to determine the dissociation of a C5-bound antibody at an acidic pH. The low-pH dissociation factor may be measured by associating an antibody and an antigen (e.g. the humanized anti-C5a antibody and human C5) at pH 7.4 for 600 seconds, followed by a dissociation period of 600 seconds in a buffer at pH 5.8, and calculation of the percentage of antibody dissociated at pH 5.8 from the antigen. In some embodiments, the low-pH dissociation factor of the humanized anti-C5a antibody of the present invention is in the range of any one of about 5% to about 95%, about 10% to about 90%, about 15% to about 85%, about 20% to about 80%, about 20% to about 75%, about 20% to about 70%, about 20% to about 65%, about 20% to about 60%, about 25% to about 75%, about 25% to about 70%, about 25% to about 65%, about 25% to about 60%, about 30% to about 75%, about 30% to about 70%, about 30% to about 65%, about 30% to about 60%, about 35% to about 75%, about 35% to about 70%, about 35% to about 65%, about 35% to about 60%, about 40% to about 75%, about 40% to about 70%, about 40% to about 65%, about 40% to about 60%. In some embodiments, the low-pH dissociation factor of the humanized anti-C5a antibody is no less than about any of 95%, 90%, 85%, 80%, 75%, 70%, 65%, 60%, 55%, 50%, 45%, 40%, 35%, 30%, 25%, 20%, 15%, 10%, or 5%.

Neutral-pH dissociation factor, namely, the percentage of antibody dissociated at pH 7.4 from the antigen at 25° C., wherein the antibody is pre-bound to the antigen at pH 7.4 and can be used to determine the dissociation of a C5-bound antibody at a neutral pH. The neutral-pH dissociation factor may be measured by associating an antibody and an antigen (e.g. the humanized anti-C5a antibody and human C5) at pH 7.4 for 600 seconds, followed by a dissociation period of 600 seconds in a buffer at pH 7.4, and calculation of the percentage of antibody dissociated at pH 7.4 from the antigen. In some embodiments, the neutral-pH dissociation factor of the humanized anti-C5a antibody of the present invention is no more than about any of 20%, 8%1, 6%1, 4%1, 2%1, 10%, 9%, 8%, 7%, 6%, 5%, 4%, 3%, 2%, or 1%.

In some embodiments, the ratio of the low-pH dissociation factor over the neutral-pH dissociation factor of the humanized anti-C5a antibody of the present invention is any one of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the antibody for C5 at pH 5.8 over the percentage of dissociation of the antibody for C5 at pH 7.4 is at least 4, at least 5, or at least 6.

In some embodiments, the humanized anti-C5a antibody binds more strongly at a neutral pH (such as pH 7.4) than it does at an acidic pH (such as pH 5.8). In some embodiments, the low-pH dissociation factor of the humanized anti-C5a antibody is no less than about any of 95%, 90%, 85%, 80%, 75%, 70%, 65%, 60%, 55%, 50%4, 45%, 40%, 35%, 30%, 25%, 20%, 15%, 10%, or 5%. In some embodiments, the neutral-pH dissociation factor of the humanized anti-C5a antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the humanized anti-C5a antibody is about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the humanized anti-C5a antibody is no less than about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the humanized anti-C5a antibody is no less than 4. In some embodiments, the ratio of the low-pH dissociation factor over the neutral-pH dissociation factor of the humanized anti-C5a antibody is no less than 5. In some embodiments, the ratio of the low-pH dissociation factor over the neutral-pH dissociation factor of the humanized anti-C5a antibody is no less than 6.

Properties of the pH-Dependent Anti-C5a Antibodies

The pH-dependent humanized anti-C5a antibodies described herein are amenable for development and use as a pharmaceutical composition.

The pH-dependent humanized anti-C5a antibody described herein in some embodiments exhibit prolonged serum half-life in vivo. In some embodiments, the humanized anti-C5a antibody exhibits prolonged serum half-life in mice (including transgenic mice). In some embodiments, the humanized anti-C5a antibody exhibits prolonged serum half-life in other test animals. Exemplary test animals include but are not limited to, rats, chickens, rabbits, sheep, and cyno monkeys. In some embodiments, the humanized anti-C5a antibody exhibits prolonged serum half-life in humans. In some embodiments, the humanized anti-C5a antibody has a serum half-life in humans of any one of at least about 2 hours, about 3 days, about 5 days, about 7 days, about 9 days, about 11 days, about 13 days, about 15 days, about 17 days, about 19 days, about 21 days, about 23 days, about 25 days. In some embodiments, the humanized anti-C5a antibody has a serum half-life in humans that is at least about 25 days.

In some embodiments, the pH-dependent humanized anti-C5a antibody has comparable binding affinity to human C5 to benchmark anti-C5a antibodies.

### III. Pharmaceutical Compositions

Further provided by the present application are pharmaceutical compositions comprising any one of the humanized anti-C5a antibodies s and a pharmaceutically acceptable carrier. Pharmaceutical compositions can be prepared by mixing the humanized anti-C5a antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

In some embodiments, the pharmaceutical composition further comprises additional ingredients. Additional ingredients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

Additional excipients include agents which can serve as one or more of the following: (1) bulking agents, (2) solubility enhancers, (3) stabilizers and (4) and agents preventing denaturation or adherence to the container wall.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Compositions for sustained release or implantation may comprise pharmaceutically acceptable polymeric or hydrophobic materials such as an emulsion, an ion exchange resin, a sparingly soluble polymer, or a sparingly soluble salt. Suitable examples of sustained-release preparations include semi-permeable matrices of solid hydrophobic polymers containing the antagonist, which matrices are in the form of shaped articles, e.g. films, or microcapsules.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

The formulations of the pharmaceutical compositions may be prepared by any method known or hereafter developed in the art of pharmacology. Preparations include but are not limited to, bringing the active ingredient into association with a carrier or one or more other accessory ingredients, and then, if necessary or desirable, shaping or packaging the product into a desired single- or multi-dose unit. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

### IV. Methods of Use

Also provided herein are methods of inhibiting complement activation and treating diseases (such as complement-mediated diseases or disorders) in an individual by administering an effective amount of the humanized anti-C5a antibody to the individual. In some embodiments, the individual is a human.

The humanized anti-C5a antibody can be used in combination with other treatment modalities, such as, for example anti-inflammatory therapies, and the like. Examples of anti-inflammatory therapies that can be used in combination with the methods of the invention include, for example, therapies that employ steroidal drugs, as well as therapies that employ non-steroidal drugs.

In some embodiments, there is provided a method of inhibiting complement activation in an individual, comprising administering (such as systemically administering, for example by subcutaneous or intravenous administration) to the individual an effective amount of a humanized anti-C5a antibody. In some embodiments, the humanized anti-C5a antibody comprises a VH comprising I48M, D54E, and N56W mutations in reference to SEQ ID NO:1 and a VL comprising D28E and D30F mutations in reference to SEQ ID NO:2. In some embodiments, the humanized anti-C5a antibody further comprises an F29H mutation in the VH in reference to SEQ ID NO:1 and a Y96H mutation in the VL in reference to SEQ ID NO:2. In some embodiments, the humanized anti-C5a antibody further comprises a mutation selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system. In some embodiments, the humanized anti-C5a antibody further comprises an IgG4 Fc region (such as an IgG4 Fc region comprises PLA mutation: S228P, M428L, and N434A). In some embodiments, the humanized anti-C5a antibody binds more strongly at a neutral pH (such as pH 7.4) than it does at an acidic pH (such as pH 5.8). In some embodiments, the low-pH dissociation factor of the humanized anti-C5a antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the humanized anti-C5a antibody is no more than about any of 20%, 8%1, 6%1, 4%1, 12%, 10%, 9%, 8%, 7%, 6%, 5%, 4%, 3%, 2%, or 1%. In some embodiments, the ratio of the low-pH dissociation factor over the neutral-pH dissociation factor of the humanized anti-C5a antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the antibody for C5 at pH 5.8 over the percentage of dissociation of the antibody for C5 at pH 7.4 is 6 or more. In some embodiments, the humanized anti-C5a antibody is administered by subcutaneous administration.

In some embodiments, the humanized anti-C5a antibody inhibits complement activation by at least about any of 10%, 20%, 30%, 40%, 50%, 60%, 70%, 80%, 90%, or more.

In some embodiments, the humanized anti-C5a antibody inhibits C5a binding to C5aR with an IC₅₀ value of about 0.01 nM, about 0.1 nM, about 0.5 nM, about 1 nM, about 2 nM, about 3 nM, about 4 nM, about 5 nM, about 6 nM, about 7 nM, about 8 nM, about 9 nM, about 10 nM, about 20 nM, about 50 nM, about 100 nM, or above 100 nM. In some embodiments, there is provided a humanized anti-C5a antibody, wherein the humanized anti-C5a antibody inhibits C5a binding to C5aR with an IC₅₀ value of about 1 nM to about 10 nM.

In some embodiments, the humanized anti-C5a antibody inhibits C5a-induced calcium influx into the cells with an IC₅₀ value of about 0.001 nM, about 0.005 nM, about 0.01 nM, about 0.05 nM, about 0.1 nM, about 0.15 nM, about 0.2 nM, about 0.25 nM, about 0.3 nM, about 0.4 nM, about 0.5 nM, about 0.6 nM, about 0.7 nM, about 0.8 nM, about 0.9 nM, about 1 nM, or above 1 nM. In some embodiments, there is provided a humanized anti-C5 antibody, wherein the humanized anti-C5a antibody inhibits C5a-induced calcium influx into the cells with an IC₅₀ value of about 0.01 nM to about 0.3 nM.

In some embodiments, the humanized anti-C5a antibody binds to human C5 with an affinity of about 0.001 nM, about 0.005 nM, about 0.01 nM, about 0.05 nM, about 0.1 nM, about 0.15 nM, about 0.2 nM, about 0.25 nM, about 0.3 nM, about 0.4 nM, about 0.5 nM, about 0.6 nM, about 0.7 nM, about 0.8 nM, about 0.9 nM, about 1 nM. In some embodiments, the humanized anti-C5a antibody binds to human C5 with a low-nanomolar affinity. In some embodiments, there is provided a humanized anti-C5 antibody, wherein the humanized anti-C5a antibody binds to human C5 with an affinity of about 0.01 nM to about 1 nM.

In some embodiments, there is provided a humanized anti-C5 antibody, wherein the humanized anti-C5a antibody does not impact C5-mediated complement activities.

In some embodiments, there is provided a humanized anti-C5 antibody, wherein the humanized anti-C5a antibody does not inhibit C5 convertase-mediated activity.

In some embodiments, the humanized anti-C5a antibody reduces serum C5 level in a subject up to 1, 2, 3, 4, 5, 6, 7, 8, 9, 10, 11, 12, 13, 14, 15, 16, 17, 18, 19, or 20 days after administration. In some embodiments, the humanized anti-C5a antibody reduces at least 5%, 10%, 20%, 30%, 40%, 50%, 60%, 70%, 80% or 90% serum C5 level in a subject up to 15 days after administration. In some embodiments, there is provided a humanized anti-C5 antibody, wherein the humanized anti-C5a antibody reduces at least 50% of the serum C5 level in a subject up to 15 days after administration.

In some embodiments, there is provided a humanized anti-C5 antibody, wherein the humanized anti-C5a antibody inhibits C5a-induced cell migration. In some embodiments, the anti-C5a antibody inhibits C5-induced cell migration with a low-nanomolar IC₅₀. In some embodiments, the IC₅₀ value of anti-C5a antibody inhibition of C5a-induced cell migration is about 0.01 nM, about 0.05 nM, 0.1 nM, 0.5 nM, 1 nM, 5 nM, 10 nM, or above 10 nM. In some embodiments, the IC₅₀ anti-C5a antibody inhibition of C5a-induced cell migration is between 0.01 nM to 0.2 nM.

In some embodiments, there is provided a method of inhibiting complement activation in an individual, comprising administering (such as systemically administering, for example by subcutaneous or intravenous administration) to the individual an effective amount of a humanized anti-C5a antibody. In some embodiments, the humanized anti-C5a antibody comprises a VH comprising I48M, D54E, and N56W mutations in reference to SEQ ID NO:1 and a VL comprising D28E and D30F mutations in reference to SEQ ID NO:2. In some embodiments, the humanized anti-C5a antibody further comprises an F29H mutation in the VH in reference to SEQ ID NO:1 and a Y96H mutation in the VL in reference to SEQ ID NO:2. In some embodiments, the humanized anti-C5a antibody further comprises a mutation selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system. In some embodiments, the humanized anti-C5a antibody further comprises an IgG4 Fc region (such as an IgG4 Fc region comprises PLA mutation: S228P, M428L, and N434A). In some embodiments, the humanized anti-C5a antibody binds more strongly at a neutral pH (such as pH 7.4) than it does at an acidic pH (such as pH 5.8). In some embodiments, the low-pH dissociation factor of the humanized anti-C5a antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the humanized anti-C5a antibody is no more than about any of 20%, 8%1, 6%1, 4%1, 2%1, 10%, 9%, 8%, 7%, 6%, 5%, 4%, 3%, 2%, or 1%. In some embodiments, the ratio of the low-pH dissociation factor over the neutral-pH dissociation factor of the humanized anti-C5a antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more.

In some embodiments, the complement-mediated disease or disorder, particularly the disease where neutrophil activation plays pathogenic roles, is at least selected from the group consisting of: macular degeneration (MD), age-related macular degeneration (AMD), ischemia reperfusion injury, arthritis, rheumatoid arthritis, asthma, allergic asthma, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, myasthenia gravis, neuromyelitis optica, (NMO), multiple sclerosis, delayed graft function, antibody-mediated rejection, atypical hemolytic uremic (aHUS) syndrome, central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, antiphospholipid antibody-induced pregnancy loss, COVID-19, bullous pemphigoid, hidradenitis suppurativa, dermatitis herpetiformis, sweets syndrome, pyoderma gangrenosum, palmo-plantar pustulosis & pustular psoriasis, rheumatoid neutrophilic dermatoses, subcorneal pustular dermatosis, bowel-associated dermatosis-arthritis syndrome, neutrophilic eccrine hidradenitis, and linear IgA disease, or any combinations thereof.

### Dosage and Routes of Administration

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 g to about 1 mg per kilogram of body weight of the subject.

In some embodiments, the humanized anti-C5a antibody is administered for a single time. In some embodiments, the humanized anti-C5a antibody is administered for multiple times (such as any of 2, 3, 4, 5, 6, or more times). In some embodiments, the humanized anti-C5a antibody is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly. The frequency of the dose will be readily apparent to the skilled artisan and will depend upon any number of factors, include but not limited to, the type and severity of the disease being treated, the type and age of the subject, etc.

The humanized anti-C5a antibody of the present application, including but not limited to reconstituted and liquid formulations, are administered to an individual in need of treatment with the humanized anti-C5a antibodies, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, ophthalmic, rectal, vaginal, parenteral, pulmonary, buccal, intraocular or inhalation routes. Other contemplated formulations include projected nanoparticles, liposomal preparations, resealed erythrocytes containing the active ingredient, and immunologically-based formulations. Parenteral administration of the humanized anti-C5a antibody includes any route of administration characterized by physical breaching of a tissue of an individual and administration of the pharmaceutical composition through the breach in the tissue. Parental administration can be local, regional or systemic. Parenteral administration thus includes, but is not limited to, administration of the humanized anti-C5a antibody by injection of the composition, by application of the composition through a surgical incision, by application of the humanized anti-C5a antibody through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

The humanized anti-C5a antibody of the invention may be prepared, packaged, or sold in bulk, as a single unit dose, or as a plurality of single unit doses. A unit dose is discrete amount of the humanized anti-C5a antibody comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

### V. Methods of Preparation

The present application also provides isolated nucleic acids encoding the humanized anti-C5a antibodies, vectors and host cells comprising such isolated nucleic acids, and recombinant methods for the production of the humanized anti-C5a antibodies.

### Expression Vectors and Cells Producing Antibodies

In some embodiments, the invention is a cell or cell line (such as host cells) that produces at least one of the humanized anti-C5a antibodies described herein. In one embodiment, the cell or cell line is a genetically modified cell that produces at least one of the humanized anti-C5a antibodies described herein. In one embodiment, the cell or cell line is a hybridoma that produces at least one of the humanized anti-C5a antibodies thereof described herein.

Hybrid cells (hybridomas) are generally produced from mass fusions between murine splenocytes, which are highly enriched for B-lymphocytes, and myeloma “fusion partner cells” (Alberts et al., Molecular Biology of the Cell (Garland Publishing, Inc. 1994); Harlow et al., Antibodies. A Laboratory Manual (Cold Spring Harbor Laboratory, Cold Spring Harbor, 1988). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are referred to as monoclonal antibodies.

Also provided are nucleic acids encoding any of the humanized anti-C5a antibodies thereof disclosed herein, as well as vectors comprising the nucleic acids. Thus, the humanized anti-C5a antibodies of the invention can be generated by expressing the nucleic acid in a cell or a cell line, such as the cell lines typically used for expression of recombinant or humanized immunoglobulins. Thus, the antibodies and fragments of the invention can also be generated by cloning the nucleic acids into one or more expression vectors, and transforming the vector into a cell line such as the cell lines typically used for expression of recombinant or humanized immunoglobulins.

The genes encoding the heavy and light chains of the humanized anti-C5a antibodies thereof can be engineered according to methods, including but not limited to full length chemical gene synthesis, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains, or fragments thereof, can be cloned from an antibody secreting cell's genomic DNA, or cDNA is produced by reverse transcription of the RNA of the cell. Cloning is accomplished by conventional techniques including the use of PCR primers that hybridize to the sequences flanking or overlapping the genes, or segments of genes, to be cloned.

Nucleic acids encoding the humanized anti-C5a antibodies described herein, or the heavy chain or light chain or fragments thereof, can be obtained and used in accordance with recombinant nucleic acid techniques for the production of the specific immunoglobulin, immunoglobulin chain, or a fragment or variant thereof, in a variety of host cells or in an in vitro translation system. For example, the antibody-encoding nucleic acids, or fragments thereof, can be placed into suitable prokaryotic or eukaryotic vectors, e.g., expression vectors, and introduced into a suitable host cell by an appropriate method, e.g., transformation, transfection, electroporation, infection, such that the nucleic acid is operably linked to one or more expression control elements, e.g., in the vector or integrated into the host cell genome.

In some embodiments, the heavy and light chains, or fragments thereof, can be assembled in two different expression vectors that can be used to co-transfect a recipient cell. In some embodiments, each vector can contain two or more selectable genes, one for selection in a bacterial system and one for selection in a eukaryotic system. These vectors allow for the production and amplification of the genes in a bacterial system, and subsequent co-transfection of eukaryotic cells and selection of the co-transfected cells. The selection procedure can be used to select for the expression of antibody nucleic acids introduced on two different DNA vectors into a eukaryotic cell.

Alternatively, the nucleic acids encoding the heavy and light chains, or fragments thereof, may be expressed from one vector. Although the light and heavy chains are coded for by separate genes, they can be joined, using recombinant methods. For example, the two polypeptides can be joined by a synthetic linker that enables them to be made as a single protein chain in which the VL and VH regions pair to form monovalent molecules (known as single chain Fv (scFv); see e.g., Bird et al., 1988, Science 242: 423-426; and Huston et al., 1988, Proc. Natl. Acad. Sci. USA 85:5879-5883).

The invention provides for an isolated nucleic acid molecule comprising a nucleic acid sequence encoding a heavy chain and/or a light chain, as well as fragments thereof. A nucleic acid molecule comprising sequences encoding both the light and heavy chain, or fragments thereof, can be engineered to contain a synthetic signal sequence for secretion of the antibody, or fragment, when produced in a cell. Furthermore, the nucleic acid molecule can contain specific DNA links which allow for the insertion of other antibody sequences and maintain the translational reading frame so to not alter the amino acids normally found in antibody sequences. Exemplary nucleic acids sequences are set for in SEQ ID Nos: 33-62.

In accordance with the present invention, antibody-encoding nucleic acid sequences can be inserted into an appropriate expression vector. In various embodiments, the expression vector comprises the necessary elements for transcription and translation of the inserted antibody-encoding nucleic acid so as to generate recombinant DNA molecules that direct the expression of antibody sequences for the formation of an antibody, or a fragment thereof.

The antibody-encoding nucleic acids, or fragments thereof, can be subjected to various recombinant nucleic acid techniques known to those skilled in the art such as site-directed mutagenesis.

A variety of methods can be used to express nucleic acids in a cell. Nucleic acids can be cloned into a number of types of vectors. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a murine stem cell virus (MSCV) vector is used to express a desired nucleic acid. MSCV vectors have been demonstrated to efficiently express desired nucleic acids in cells. However, the invention should not be limited to only using a MSCV vector, rather any retroviral expression method is included in the invention. Other examples of viral vectors are those based upon Moloney Murine Leukemia Virus (MoMuLV) and HIV. In some embodiments, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193).

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

A promoter and/or enhancer that effectively directs the expression of the DNA segment in the cell type, organelle, and organism chosen for expression may be employed. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation and the like. Methods for producing cells comprising vectors and/or exogenous nucleic acids are well-known in the art. See, for example, Sambrook et al. (2012) and Ausubel et al. (1999).

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### 1. Vector Construction

Polynucleotide sequences encoding polypeptide components of the humanized anti-C5a antibody of the present application can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector described herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the −galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this application should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments, the signal sequences used in both cistrons of the expression system are STII signal sequences or variants thereof.

In some embodiments, the production of the humanized anti-C5a antibodies can occur in the cytoplasm of the host cell, and therefore does not require the presence of secretion signal sequences within each cistron. In some embodiments, polypeptide components, such as the polypeptide encoding the VH domain of the first antigen binding portion optionally fused to the second antigen binding portion, and the polypeptide encoding the V_{L} domain of the first antigen binding portion optionally fused to the second antigen binding portion, are expressed, folded and assembled to form functional humanized anti-C5a antibodies within the cytoplasm. Certain host strains (e.g., the E. coli trxB⁻ strains) provide cytoplasm conditions that are favorable for disulfide bond formation, thereby permitting proper folding and assembly of expressed protein subunits. Proba and Pluckthun Gene, 159:203 (1995).

### 2. Protein Production in Prokaryotic Host Cells.

Prokaryotic host cells suitable for expressing the humanized anti-C5a antibodies of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A (nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the humanized anti-C5a antibodies of the present application are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. In some embodiments, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed humanized anti-C5a antibodies of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermenters use agitator impellers to distribute oxygen and nutrients, especially glucose (the preferred carbon/energy source). Small scale fermentation refers generally to fermentation in a fermenter that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the humanized anti-C5a antibodies of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the humanized anti-C5a antibodies of the present application.

### 3. Protein Production in Eukaryotic Cells

In some embodiments, the humanized anti-C5a antibodies described herein can be expressed in eukaryotic cells. For eukaryotic expression, the vector components generally include, but are not limited to, one or more of the following, a signal sequence, an origin of replication, one or more marker genes, and enhancer element, a promoter, and a transcription termination sequence.

### a) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for the humanized anti-C5a antibodies production and cultured in conventional nutrient media modified as appropriate for inducing promoters, selecting transformants, or amplifying the genes encoding the desired sequences. In some embodiments, the humanized anti-C5a antibodies are expressed in CHO cells. In some embodiments, the humanized anti-C5a antibodies are expressed in Expi-CHO cells.

### b) Culturing the Host Cells

The host cells used to produce the humanized anti-C5a antibodies of the present application may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### c) Protein Purification

The humanized anti-C5a antibodies produced herein may be further purified to obtain preparations that are substantially homogeneous for further assays and uses. Standard protein purification methods known in the art can be employed.

### 4. Antibody Production and Modification

Components of the humanized anti-C5a antibodies can be produced using any known methods in the art, including methods described below.

### a) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g, isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al, Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind the protein used for immunization. Alternatively, lymphocytes may be immunized in vitro, Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pliickthun, Immunol. Revs. 130:151-188 (1992).

In a further embodiment, antibodies can be isolated from antibody phage libraries generated using the techniques described in McCafferty et al., Nature, 348:552-554 (1990). Clackson el al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks el al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### b) Humanized Antibodies

Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domain, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Various forms of the humanized antibody are contemplated. For example, the humanized antibody may be an antibody fragment, such as an Fab, which is optionally conjugated with one or more cytotoxic agent(s) in order to generate an immunoconjugate. Alternatively, the humanized antibody may be an intact antibody, such as an intact IgG4 antibody.

### e) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993), Bruggermann et al., Year in Immuno., 7:33 (1993); U.S. Pat. No. 5,591,669 and WO 97/17852. Transgenic mice or rats capable of producing fully human antibodies are known in the art. See, e.g., US20090307787A1, U.S. Pat. No. 8,754,287, US20150289489A1, US20100122358A1, and WO2004049794.

Alternatively, phage display technology can be used to produce human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human antibodies may also be generated in vitro by activated B cells (see U.S. Pat. Nos. 5,567,610 and 5,229,275).

### d) Antibody Fragments

In certain circumstances there are advantages to using antibody fragments, such as antigen binding fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J Biochem Biophys. Method. 24:107-117 (1992); and Brennan et al., Science 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ with increase in vivo half-life is described in U.S. Pat. No. 5,869,046. In other embodiments, the antibody of choice is a single chain Fv fragment (scFv). See WO 93/16185; U.S. Pat. Nos. 5,571,894 and 5,587,458. The antibody fragment may also be a “linear antibody”, e.g., as described in U.S. Pat. No. 5,641,870. Such linear antibody fragments may be monospecific or bispecific.

### e) Effector Function Engineering

It may be desirable to modify the humanized anti-C5a antibodies of the present application with respect to Fc effector function, e.g., so as to modify (e.g., enhance or eliminate) antigen-dependent cell-mediated cytotoxicity (ADCC) and/or complement dependent cytotoxicity (CDC) of the antibody. In a preferred embodiment, Fc effector function of the humanized anti-C5a antibody is reduced or eliminated. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric humanized anti-C5a antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff el al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half-life of the antibody, one may incorporate a salvage receptor binding epitope into the humanized anti-C5a antibody as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### f) Other Amino Acid Sequence Modifications

Amino acid sequence modification(s) of the antibodies, such as single chain antibodies or antibody components of the humanized anti-C5a antibodies, described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989). Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the table below under the heading of “preferred substitutions”. If such substitutions result in a change in biological activity, then more substantial changes, denominated “exemplary substitutions” in Table 2, or as further described below in reference to amino acid classes, may be introduced and the products screened.

TABLE 2
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants to the humanized anti-C5a antibodies of the present application are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant versions.

### g) Other Modifications

The humanized anti-C5a antibodies of the present application can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

### Kits

The invention also includes a kit comprising a humanized anti-C5a antibody of the invention and an instructional material which describes, for instance, administering the humanized anti-C5a antibody to an individual as a therapeutic treatment or a non-treatment use as described elsewhere herein. In an embodiment, this kit further comprises a (optionally sterile) pharmaceutically acceptable carrier suitable for dissolving or suspending therapeutic composition, comprising a humanized anti-C5a antibody, or combinations thereof, of the invention, for instance, prior to administering the antibody to an individual. Optionally, the kit comprises an applicator for administering the antibody. Also provided are unit dosage forms comprising the humanized anti-C5a antibodies.

### EXAMPLES

The examples below are intended to be purely exemplary of the invention and should therefore not be considered to limit the invention in any way. The following examples and detailed description are offered by way of illustration and not by way of limitation.

### Example 1: Construction and Selection of the Anti-C5a Antibodies

A mouse anti-C5a IgG4 antibody/scFv comprising the VH amino acid sequence of SEQ ID NO: 1 and VL amino acid sequence of SEQ ID NO:2 was used to generate humanized anti-C5a antibodies and mutants thereof. A CDR grafting method was used to generate a humanized anti-C5a construct for further affinity optimization, wherein the CDRs of the mouse scFv was grafted onto the VH and VL framework according to SEQ ID NOs: 9 and 10, respectively. The VH and the VL of the CDR-grafting anti-C5a construct are shown in SEQ ID NOs: 84 and 85.

### 1. Affinity Optimization

Each amino acid of six complementary-determining regions (CDRs) of the anti-C5a scFv clone comprising the VH and VL sequences of SEQ ID NOs: 84 and 85 was individually mutated to all 20 amino acids using a site-directed mutagenesis method. DNA primers containing an NNS codon encoding 20 amino acids were used to introduce mutations at each targeted CDR position. The degenerate primers were used in site-directed mutagenesis reactions. Briefly, each degenerate primer was phosphorylated. The PCR condition is: 94° C. for 2 minutes, (94° C. for 30 seconds, 55° C. for 30 seconds, 72° C. for 5 minutes), 16 cycles, 72° C. for 10 minutes. PCR products were purified and then electroporated into BL21 for colony formation and production of scFv fragments. A construct denoted NM2 comprising a VH and a VL of SEQ ID NOs: 82 and 83 was selected for scFv mutant library screening.

2. Primary scFv Mutant Library Screening

The primary screening consisted of a single point scFv capture ELISA (SPE) was carried out as following: 96-well Maxisorp lmmunoplates were coated with anti-c-myc antibody (Bethyl-A190-204A} in coating buffer PBS at pH 7.4 overnight at 4° C. The next day plates were blocked with casein in PBS at pH 7.4 for 1 hour at 25° C. ScFv containing PEs were then added to the plates and incubated at 25° C. for 1 hour. After washing, biotinylated antigen was added to the well followed by incubation for 1 hour at 25° C. This was followed by incubation with SA-horseradish peroxydase (HRP) (InvitrogenSNN1004) conjugate for 1 hour at 25° C. HRP activity was detected with tetra-methylbenzidine (TMB) (KPL-5120-0047) substrate and the reaction was quenched with 2M HCl. Plates were read at 450 nM. Clones exhibiting an optical density (OD) signal at 450 nm greater than 2 fold of the parental clone were picked and sequenced.

7 sequence unique, binding improved anti-C5a scFv constructs were confirmed by SPR. The SPR binding summary is shown in Table 3.

TABLE 3
Primary screening hits summary (SPR)

| | | | Kon | Koff | KD |
|---|---|---|---|---|---|
| Analyte | Ligand | Mutation | (1/Ms) | (1/s) | (M) |
| Cyno- | NM1 | / | 2.02E+06 | 8.26E−05 | 4.09E−11 |
| molgus | NM2 (SEQ | / | 7.02E+05 | 1.33E−01 | 1.90E−07 |
| antigen | ID Nos: | | | | |
| | 82 and 83) | | | | |
| | 1 | N to F | 1.76E+06 | 1.11E−02 | 6.32E−09 |
| | 2 | N to W | 1.85E+06 | 3.86E−03 | 2.09E−09 |
| | 3 | Y to F | 1.12E+06 | 6.25E−02 | 5.59E−08 |
| | 4 | D to F | 1.30E+06 | 4.80E−02 | 3.69E−08 |
| | 5 | A to T | 1.17E+06 | 6.90E−02 | 5.87E−08 |
| | 6 | N to V | 1.11E+06 | 4.82E−02 | 4.36E−08 |
| | 7 | N to L | 1.14E+06 | 4.55E−02 | 4.00E−08 |

3. Combinatorial Screening of the Anti-C5a scFv Library

The point mutations in VH and VL determined to be beneficial for binding to antigen were further combined to gain additional binding synergy. Briefly, each degenerate primer was phosphorylated, then used in a 10:1 ratio with uridinylated ssDNA. The mixture was heated to 85° C. for 5 minutes then cooled down to 55° C. for over 1 hour. Thereafter, T4 ligase and T4 DNA polymerase were added and mix was incubated for 1.5 hours at 37° C. Typically, 200 ng of the combinatorial library DNA was electroporated into BL21 for colony formation and for production of scFv fragments.

The combinatorial mutants were expressed as a scFv and screened using the capture ELISA. Clones exhibiting an optical density (OD) signal at 450 nanometer greater than two-fold of parental clone were sequenced and further confirmed by capture ELISA and SPR.

### 4. Affinity Ranking ELISA

The affinity ranking ELISA was carried out as following: scFv concentrations in PEs were measured by Quantification ELISA using purified scFv sample as a standard. Briefly, anti-His-antibody (Genscript-A00186) were coated on the plates in coating buffer PBS at pH7.4 overnight at 4° C. The next day, plates were blocked with casein in PBS at pH7.4 for 1 hour at 25° C. ScFv containing PEs were diluted 3 fold serial dilutions and then added to the plates. A purified scFv standard sample with known concentration was used as a standard. After incubation at 25° C. for 1 hour and washing 3 times with PBS-T, anti-c-myc-HRP (Bethyl-A190-104P) was added to the wells followed by incubation for 1 hour at 25° C. HRP activity was detected with TMB substrate and the reaction was quenched with 2M HCl. Plates were read at 450 nM.

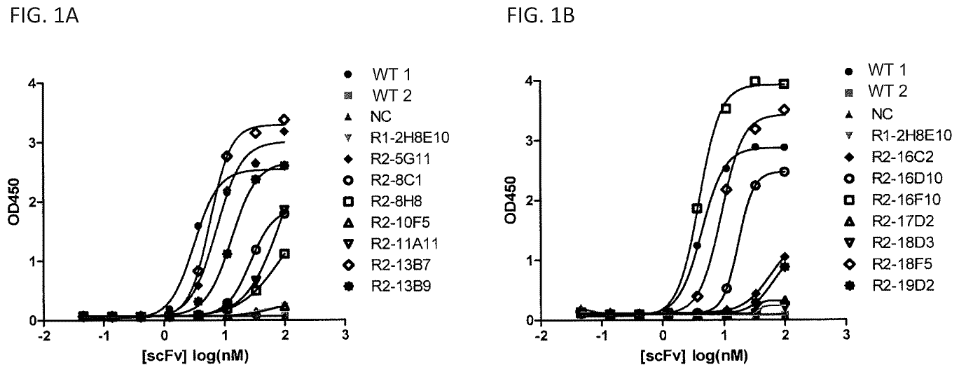

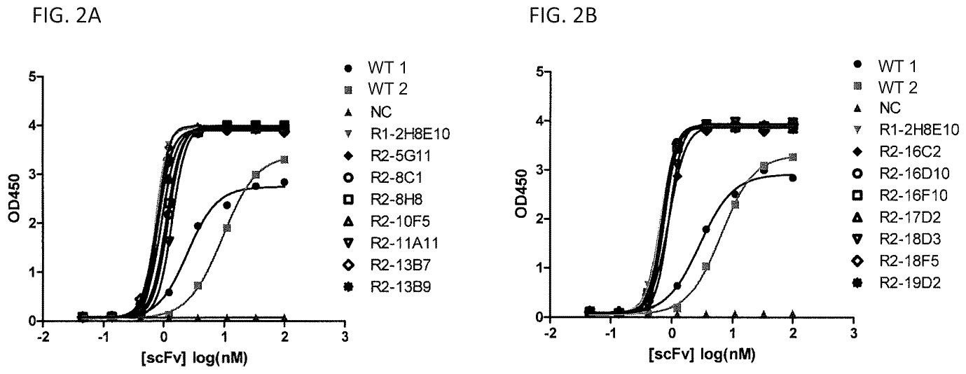

The anti-C5a cyno antigen and human antigen were coated on the plates in PBS at pH 7.4 overnight at 4° C. respectively. The next day, plates were blocked with casein in PBS for 1 hour at 25° C. Normalized scFv containing PEs were diluted using 3 fold serial dilutions starting at 100 nM, added to the plates and incubated at 25° C. for 1 hour. After washing, anti-c-myc-HRP was added to the wells followed by incubation for 1 hour at 25° C. HRP activity was detected with TMB substrate and the reaction was quenched with 2M HCl. Plates were read at 450 nM. Data were fitted into one-site binding equation using GraphPad Prism 5 software. The results of hits binding to cynomolgus antigen are shown in FIGS. 1A-1B. The results of hits binding to human antigen are shown in FIGS. 2A-2B.

### 5. SPR of Anti-C5a Hits

### Immobilization

The activator was prepared by mixing 400 mM EDC (1-ethyl-3-(3-dimethylaminopropyl) carbodiimide) and 100 mM NHS (N-hydroxysuccinimide) (GE) immediately prior to injection. The CMS sensor chip was activated for 600 s with the mixture at a flow rate of 10 μL/min. Mixture of anti-c-myc and anti his antibodies in 10 mM NaAc (pH 4.5) was then injected to Fc1 and Fc2 of channel 1 to channel 8 for 600 seconds at a flow rate of 10 μL/min. The chip was deactivated by 1 M ethanolamine-HCl (GE) at a flow rate of 10 μL/min for 420 seconds.

### Ligand Capture and Running Analyte

The anti-C5a scFv was diluted to 0.5 μg/ml, the other samples were diluted to 2 μg/ml in running buffer 1×HBS-EP+ (0.01 M HEPES, 0.15 M NaCl, 0.003 M EDTA, 0.05% surfactant P20). The same dilution formula as the scFvs was used for NC (negative control) with TES (Tris-EDTA-Sucrose) buffer. Diluted TES buffer was injected into channel Fc1 at a flow rate of 10 μL/min for 40 s, diluted scFv was injected into channel Fc2 at a flow rate of 10 μL/min for 40 s. Then 60 nM of analyte cynomolgus antigen or 5 nM of analyte human antigen and running buffer were injected into Fc1-Fc2 at a flow rate of 100 μL/min for an association phase of 60 s, followed by 1200 s dissociation. 10 mM glycine pH 1.5 as regeneration buffer was injected following every dissociation phase. The chip was regenerated with 10 mM glycine pH 1.5.

### Data Analysis

The sensorgrams for reference channel Fc1 and buffer channel were subtracted from the test sensorgrams. Data were fitted by 1:1 kinetic binding model. Molecular weight of 8.4 kDa was used to calculate the molar concentration of the cyno antigen. A molecular weight of 8.2 kDa was used to calculate the molar concentration of the human antigen. The results of binding to cyno antigen are shown in Table 4. The results of binding to cyno antigen are shown in Table

TABLE 4
Combination mutant scFv hits summary
of SPR on cynomolgus antigen

| | | kd | Capture |
|---|---|---|---|
| Analyte | Ligand | (1/s) | level (RU) |
| Cynomolgus | NM1 | 7.61E−05 | 146 |
| antigen | 16F10 (SEQ ID NOs: 56 and 57) | 1.55E−04 | 414 |
| | 13B7 (SEQ ID NOs: 58 and 59) | 2.52E−04 | 454 |
| | 5G11 (SEQ ID NOs: 60 and 61) | 2.79E−04 | 472 |
| | 18F5 (SEQ ID NOs: 62 and 63) | 3.19E−04 | 470 |
| | 13B9 (SEQ ID NOs: 64 and 65) | 3.27E−04 | 456 |
| | 16D10 (SEQ ID NOs: 9 and | 4.65E−04 | 435 |
| | 10) | | |
| | 8C1 (SEQ ID NOs: 66 and 67) | 4.81E−04 | 362 |
| | 11A11 (SEQ ID NOs: 78 and | 7.86E−04 | 274 |
| | 79) | | |
| | 8H8 (SEQ ID NOs: 76 and 77) | 9.27E−04 | 363 |
| | 19D2 (SEQ ID NOs: 74 and 75) | 1.02E−03 | 430 |
| | 16C2 (SEQ ID NOs: 72 and 73) | 1.76E−03 | 336 |
| | 17D2 (SEQ ID NOs: 68 and 69) | 1.90E−03 | 435 |
| | 10F5 (SEQ ID NOs: 70 and 71) | 2.02E−03 | 404 |
| | 2H8E10 (SEQ ID NOs: 54 and | 2.58E−03 | 420 |
| | 55) | | |
| | 18D3 (SEQ ID NOs: 80 and 81) | 4.18E−03 | 405 |
| | NM2 (SEQ ID NOs: 82 and 83) | 7.83E−02 | 323 |
| | Negative control | No binding | 112 |

TABLE 5
Combination mutant scFv hits summary of SPR on human antigen

| | | kd | Capture |
|---|---|---|---|
| Analyte | Ligand | (1/s) | level (RU) |
| Human | NM1 | 9.35E−05 | 42 |
| antigen | 16F10 (SEQ ID NOs: 56 and 57) | 1.14E−05 | 74 |
| | 13B7 (SEQ ID NOs: 58 and 59) | <1.00E−05 | 75 |
| | 5G11 (SEQ ID NOs: 60 and 61) | <1.00E−05 | 75 |
| | 18F5 (SEQ ID NOs: 62 and 63) | 3.03E−05 | 80 |
| | 13B9 (SEQ ID NOs: 64 and 65) | 2.20E−05 | 79 |
| | 16D10 (SEQ ID NOs: 9 and | 2.34E−05 | 68 |
| | 10) | | |
| | 8C1 (SEQ ID NOs: 66 and 67) | <1.00E−05 | 64 |
| | 11A11 (SEQ ID NOs: 78 and | <1.00E−05 | 67 |
| | 79) | | |
| | 8H8 (SEQ ID NOs: 76 and 77) | 2.69E−05 | 59 |
| | 19D2 (SEQ ID NOs: 74 and 75) | <1.00E−05 | 81 |
| | 16C2 (SEQ ID NOs: 72 and 73) | 2.58E−05 | 56 |
| | 17D2 (SEQ ID NOs: 68 and 69) | 1.89E−05 | 78 |
| | 10F5 (SEQ ID NOs: 70 and 71) | <1.00E−05 | 72 |
| | 2H8E10 (SEQ ID NOs: 54 and | 1.82E−05 | 72 |
| | 55) | | |
| | 18D3 (SEQ ID NOs: 80 and 81) | 2.98E−05 | 65 |
| | NM2 (SEQ ID NOs: 82 and 83) | 7.36E−04 | 60 |
| | Negative control | No binding | 42 |

16D10 comprising the VH amino acid sequence of SEQ ID NO: 9 and VL amino acid sequence of SEQ ID NO: 10 was selected for further optimization of pH-dependent antigen binding properties. Fc domain mutations were introduced into 16D10. The designed mutations targeted positions were introduced by QuikChange® (Agilent) or Q5® Site-Directed Mutagenesis (NEB).

To introduced pH dependent binding, Histidine substitution was introduced to each of positions in the 6 CDR regions of 16D10 by QuikChange® mutagenesis, a method known as Histidine scanning. For each variant, HEK293 cells were transfected with the expression plasmids carrying a mutated 16D10 heavy chain and a wild type light chain transiently, and cultivated with serum-free medium in shaking flasks for 5-7 days. The light chain variants were obtained similar way by co-transfected with a mutated light chain and a wild type heavy chain. The expression supernatant was collected by centrifugation for binding characterization. IgG from the expression supernatant was used to measure C5 binding under pH 7.4 and pH 5.8 respectively using Gator™ (Probe Life, Inc.).

Mutant antibodies that maintained the same binding dissociation constant at pH 7.4 but reduced binding dissociation constant at pH 5.8 compared to 16D10 were selected. The pH differential binding variants with multiple Histidine mutations were created by co-transfection of binding beneficial, single Histidine mutation in heavy or light chains. Variants with multiple binding beneficial Histidine mutations within a chain were created by QuikChange® mutagenesis of the beneficial mutations into same chain. The pH binding differential variants were further purified from the expression supernatant by Protein-A affinity chromatography for affinity determinations. Purified protein was dialyzed into 20 mM histidine buffer with 150 mM NaCl and quantitated by Nanodrop (Thermo Fisher Scientific, Inc., USA) before affinity measurement using Gator™ (Probe Life, Inc.).

Three mutant humanized, binding affinity matured and pH dependent C5a binding constructs were identified for further characterization: E54H (VH: SEQ ID NO: 17; VL: SEQ ID NO: 18), comprising an F29H mutation in VH CDR1, an E54H mutation in VH CDR2, and a Y96H mutation in VL CDR3; N97H (VH: SEQ ID NO: 25; VL: SEQ ID NO: 26), comprising an F29H mutation in VH CDR1, an N97H mutation in VH CDR3, and a Y96H mutation in VL CDR3; N92H (VH: SEQ ID NO: 33; VL: SEQ ID NO: 34), comprising an F29H mutation in VH CDR1, an N92H mutation in VL CDR2, and a Y96H mutation in VL CDR3. The pH-dependent data are described in Example 4.

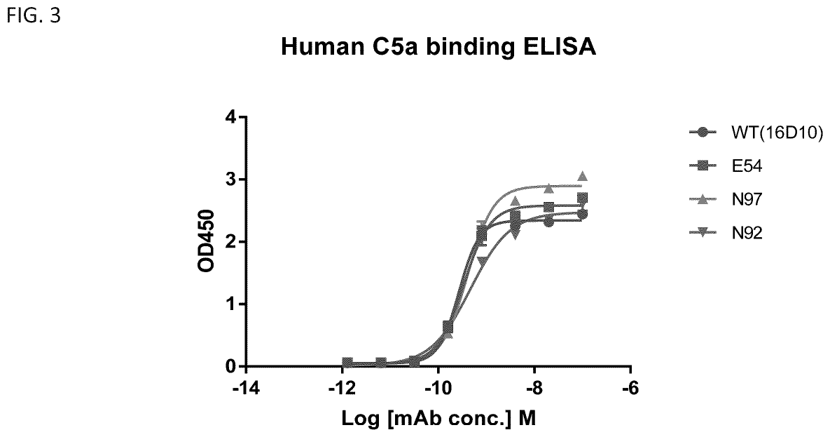

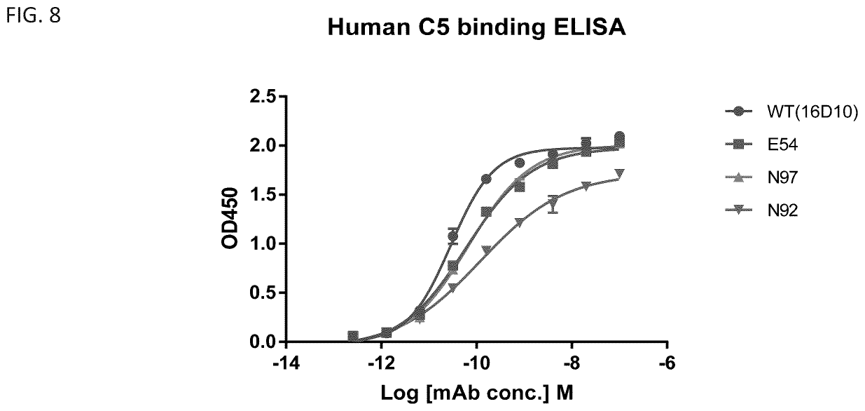

The humanized anti-C5a antibodies were tested for their affinities to human C5a by ELISA. FIG. 3 shows that all humanized anti-C5a constructs (16D10, E54H, N97H, and N92H) bound to human C5a with comparable high affinity. The antibodies bound to C5 with a higher affinity (FIG. 8).

Minimizing disruption of C5-mediated pathways is desired for anti-C5a constructs, as C5 presents a profound sink to C5a-targeting drugs. A classical pathway complement-mediated sheep red blood cell lysis assay was used to assess the C5 inhibitory effect of all anti-C5a antibody constructs.

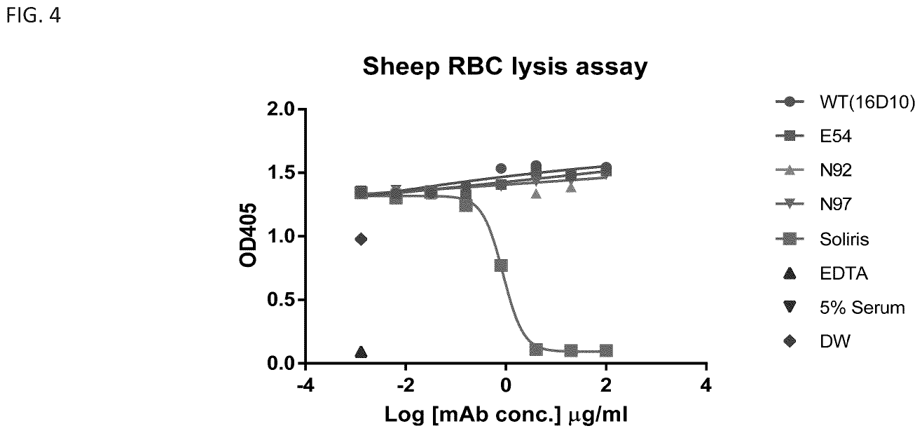

Antibody-sensitized sheep RBCs (2×10⁷ cells/assay, Solarbio) were incubated at 37° C. for 30 min with 5% normal human serum (NHS, from Quidel) in gelatin veronal buffer (GVB2+, Complement Technology nc; total assay volume: 50 μl). Anti-C5a constructs were incubated with NHS for 1 hour at 4° C. before addition into the sheep RBCs at various concentrations (FIG. 4). The lysis reactions were stopped by addition of 40 mM EDTA in ice-cold PBS. The incubation mixtures were centrifuged for 5 min at 1500 rpm. The supernatant from each mixture was collected and measured for OD at 405 nm. EDTA, distilled water (denoted DW), and an anti-C5 benchmark antibody Soliris were used as positive controls for RBC lysis. 5% serum was used as a negative control.

The results of sheep RBC lysis assay are shown in FIG. 4 and Table 6. Positive controls (EDTA, water, and Soliris) lysed sheep RBC; Soliris effectively blocks sheep RBC lysis with an IC50 of 1.23 μg/mL The humanized anti-C5a constructs did not show inhibition in lysis, suggesting minimal disruption of C5-mediated pathways.

TABLE 6
Sheep RBC lysis assay results

| Ab ID | IC50 (μg/ml) |
|---|---|
| E54H | Not active |
| N97H | Not active |
| N92H | Not active |
| 16D10 | Not active |
| Soliris | 1.23 |

### Example 2: In Vitro Functional Assays

### 1. Ligand Blocking Assay in U937 Cells

U937/C5aR cells were washed twice by PBS and suspended in assay buffer (PBS+0.1% BSA) at the density of 3×10⁶ cells/ml. Add 100 μl cells to 96 well microplate, 50 μl compound diluted in assay buffer and 50 μl biotinylated ligand human C5a (final concentration is 20 nM) were added to corresponding wells in order, plate was incubated on ice for 120 min. 1000 rpm centrifuge for 3-5 min at 4° C., remove supernatant and cells were washed by pre-cold PBS twice. Add 100 μl FITC conjugated streptavidin (eBioscience) to cells and incubate on ice for another 30 min. Centrifuge at 4° C., remove supernatant and cells were washed by pre-cold PBS twice. Add 150 μl 0.5% PFA to suspend cells and detect the signal by FACS (Beckman, Cytoflex). IC₅₀ value was calculated by GraphPad Prism software. High control means cells were incubated with biotinylated C5a without adding anti-C5a constructs; Low control means cells were incubated with assay buffer without adding anti-C5a constructs.

The four anti-C5a constructs (16D10, E54H, N97H, and N92H) were evaluated for their C5a receptor (C5aR) blocking capacities in U937 cells.

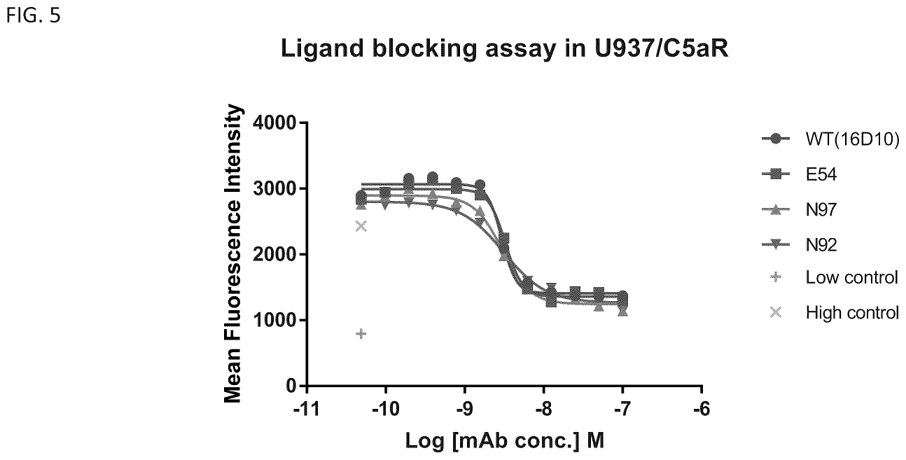

Anti-C5a constructions were titrated into U937 cells expressing C5aR at series of concentrations shown in FIG. 5. Mean fluorescence intensities were measured at each antibody concentration. U937 cells without addition of antibodies was used as the low control. Cells incubated with biotinylated C5a without addition of anti-C5a constructs were used as the high control.

The experiments were repeated. Representative results are shown in FIG. 5 and summarized in Table 7. All four anti-C5a constructs (16D10, E54H, N97H, and N92H) showed potent ligand blocking at nanomolar concentrations.

TABLE 7
Ligand blocking in U937 cells by humanized anti-C5a antibodies

IC50(nM)

| Antibody | Experiment #1 | Experiment #2 |
|---|---|---|
| 16D10 | 2.029 | 5.897 |
| E54H | 2.566 | 6.53 |
| N97H | 4.711 | 5.963 |
| N92H | 3.679 | 6.229 |

### 2. C5aR Antagonist Migration Assay

Blocking of C5aR has been shown to inhibit chemotaxis of lymphatic cells. U937 cells stably transfected with the C5a receptor (U937-C5aR cells) were seeded onto the upper chambers of 96-well Transwell inserts, at 3×10⁵ cells per well in RPMI media with 0.5% BSA. The Transwell inserts had 3.0-μm pore size polycarbonate membrane filters (Corning). The lower Boyden chambers received 10 nM of recombinant human complement C5a pretreated with antibody in RPMI media with 0.5% BSA. After a 3-hour incubation at 37° C., the migrated cells in the lower chambers were lysed by adding 50 μl CellTiter-Glo (Promega) and luminescence intensity (LI) was read by microplate reader (BioTek). Percentage of inhibition was calculated as follows: % inhibition=[1−(LI−LI_{[low control]})/(LI_{[high control]}−LI_{[low control]})]*100. IC₅₀ value was calculated by GraphPad Prism software. High control is adding C5a only in the lower chamber; Low control is adding assay buffer only in the lower chamber.

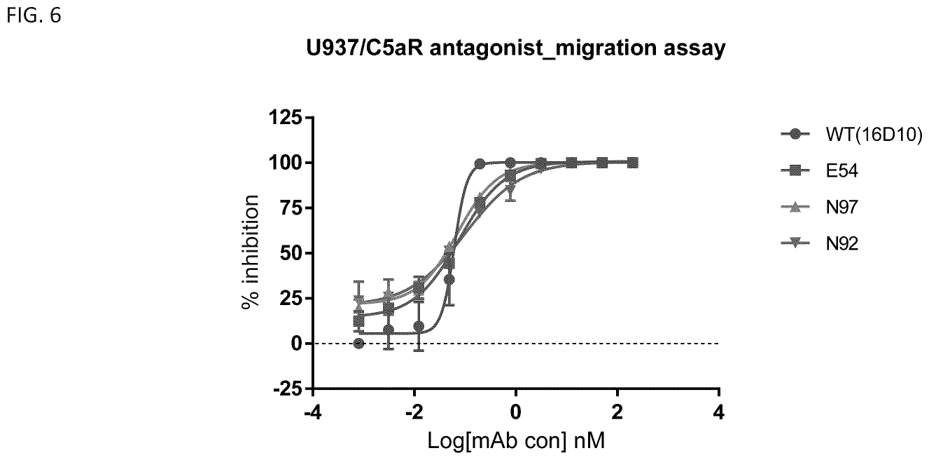

Table 8 and FIG. 6 show that all four anti-C5a constructs (16D10, E54H, N97H, and N92H) effectively inhibit migration of U937 cells at low nanomolar concentrations with 2 independent experiments.

TABLE 8
Inhibition of cell migration

| Ab ID | IC₅₀ (nM) |
|---|---|
| E54H | 0.076 |
| N97H | 0.07 |
| N92H | 0.099 |
| 16D10 | 0.061 |

### 3. C5aR Antagonist FLIPR Assay

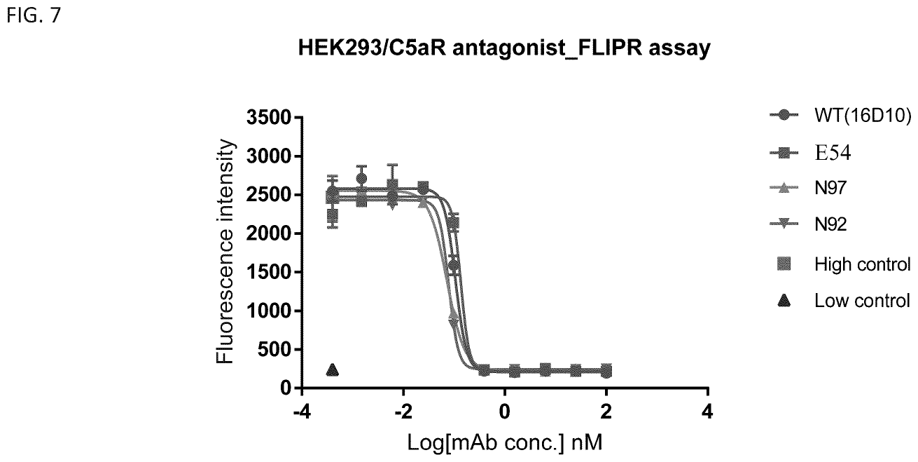

The humanized anti-C5a constructs were tested for their inhibitory activities on C5a-induced calcium influx using FLIPR assays. The anti-C5a constructs were titrated into HEK293 cells prepared according to the instructions of a FLIPR assay kit. Briefly, cells were suspended in growth media at the density of 1×10⁶ cells/ml. Seed 20 μl cell suspension to the 384-well plate and culture for overnight. Transfer 250 nl anti-C5a construct solution to the cell plate using Echo, incubate for 60 min. Cells were added with Fluo-4 Direct™ dye and incubate for 50 min at 37° C. 5% CO2 and followed by 10 min at room temperature. Place the cell plate into FLIPRTETRA (Molecular Devices). Transfer 10 μl of agonist human C5a to the cell plates. Fluorescence was measured at each antibody concentration point shown in FIG. 7. IC50 was calculated by GraphPad Prism software. Results are shown in FIG. 7 and Table 9. A sample with 250 nl assay buffer added instead of anti-C5a construct was used as a high control.

TABLE 9
Inhibition of calcium influx by humanized anti-C5a antibodies

IC50(nM)

| Antibody | Experiment #1 | Experiment #2 |
|---|---|---|
| 16D10 | 0.106 | 0.135 |
| E54H | 0.136 | 0.154 |
| N97H | 0.072 | 0.138 |
| N92H | 0.078 | 0.145 |

All four humanized anti-C5a constructs inhibited calcium influx into HEK293 cells at low nanomolar concentrations.

### Example 3: C5 Binding and the “Sink Effect”

The anti-C5a constructs were further tested for their affinities to human C5 using ELISA. Off-target C5 binding reduces availability of anti-C5a antibodies (C5-mediated “sink effect”). 4 humanized anti-C5a constructs (16D10, E54, N92 and N97) were serial diluted and added to C5 pre-coated plate, OD450 was measured by microplate reader at 450 nm. Each data point was the mean of 2 replicates. Results are shown in FIG. 8 and Table 10.

Despite having shown minimal impact on sheep RBC lysis (Example 1), all anti-C5a constructs (including benchmarks) also bound to human C5 at neutral pH. E54H, N97H, and N92H mutants showed more selective binding to C5a than to C5 than 16D10 (increased C5a/C5 binding ratio comparing to 16D10).

TABLE 10
Affinities to Human C5 determined by ELISA

| Ab ID | huC5 (nM) | huC5a (nM) | Fold affinity (C5/C5a) |
|---|---|---|---|
| E54H | 0.06011 | 0.3364 | 5.6 |
| N97H | 0.0682 | 0.4083 | 6.0 |
| N92H | 0.1203 | 0.4678 | 3.9 |
| WT (16D10) | 0.02883 | 0.2694 | 9.3 |

### Example 4: pH-Dependent C5 Binding and Reduced “Sink Effect”

pH dependency of the humanized anti-C5a constructs were tested using C5 binding assays. A Biolayer interferometry instrument, Gator™ (Probe life Inc., USA) was used to determine pH dependent dissociation of histidine mutants and the benchmark antibodies. Briefly, anti-human IgG Fc (FHC) Probes were equilibrated in the kinetic (K) buffer of Phosphate-buffered saline containing 0.02% bovine serum albumin and 0.002% Tween20 pH 7.4. Antibodies were captured onto the sensors by dipping them into 200 μL of transfection supernatant for 600 s at pH 7.4. The biosensors were then incubated with human C5 prepared in the K buffer pH 7.4 (40 nM) for 600s followed by 600-second dissociation period in K Buffer, pH 7.4 or pH 5.8. The data were processed and analyzed by the Gator evaluation software.

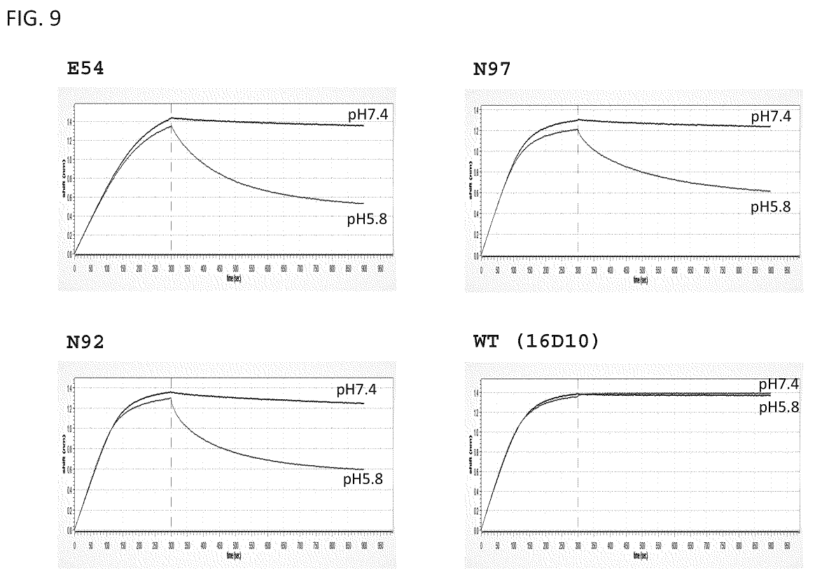

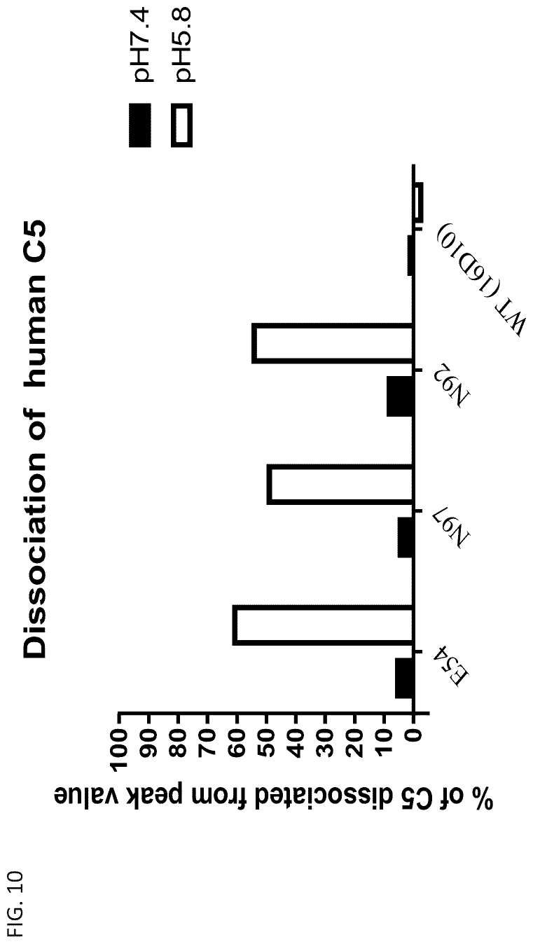

Binding curves of 16D10, E54H, N97H, and N92H are shown in FIG. 9. Percentage dissociation values at the end of 600 s are shown in FIG. 10 and summarized in Table 11 below.

As shown in FIG. 9, FIG. 10 and Table 11, the histidine mutation constructs E54H, N97H, and N92H displayed significant pH-dependent binding to human C5, with 49%-61% dissociation values at pH 5.8. For E54H, N97H, and N92H, the ratios between percentage dissociation at pH 5.8 and pH 7.4 were 11.3, 10.9, and 6.54, respectively. 16D10 and benchmarks did not show pH dependent binding to human C5. These results demonstrated that the histidine mutation constructs could potentially circumvent the “sink effect” and have greater persistence in vivo, because immune complexes (i.e., an anti-C5a antibody bound to C5 and thereby subjected to the “sink effect”) taken up by cells will dissociate in the acidic environment of the endosome and allow the freed antibody to be recycled back out of the cell through the neonatal Fc receptor (FcRn) where it is available to bind to a new C5a molecule.

TABLE 11
pH dependent human C5 binding by Biolayer interferometry

| | Dissociation | Dissociation | Ratio between dissociation |
|---|---|---|---|
| Ab ID | pH 5.8 (%) | pH 7.4 (%) | at pH 5.8 and 7.4 |
| E54H | 60.8 | 5.39 | 11.3 |
| N97H | 49.0 | 4.49 | 10.9 |
| N92H | 54.2 | 8.29 | 6.54 |
| 16D10 | −2.42 | 1.16 | −2.09 |

### Example 5: In Vivo Pharmacokinetics Evaluation of the Anti-C5a Constructs

In vivo pharmacokinetics of the anti-C5a constructs were tested in C5 humanized mice on SCID/human FcRn transgenic background (denoted as HuC5/Scid/FcRn, human FcRn transgenic and mouse FcRn knockout). N97H, N92H, and 16D10 were tested.

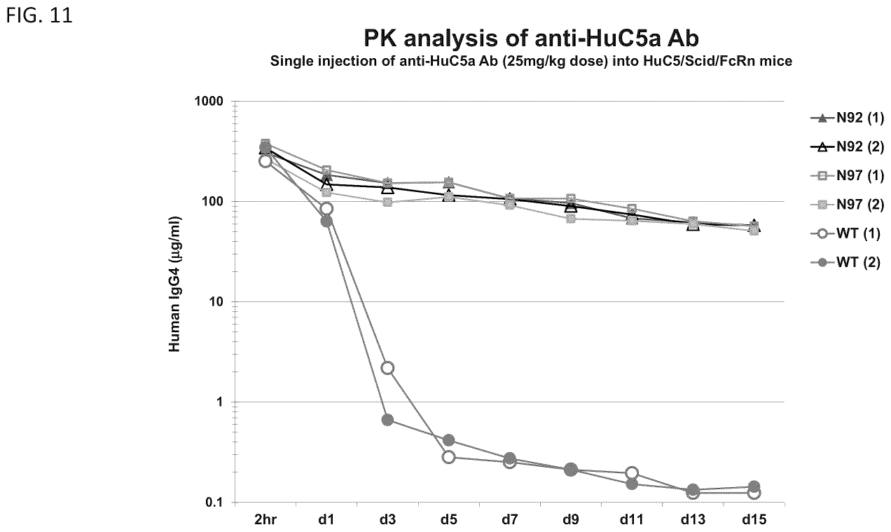

Human IgG4 in mice treated with the anti-C5a antibodies was detected using sandwich ELISA. 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with diluted plasma samples in blocking solution at RT for 1 hr. After washing, the plates were incubated with anti-human IgG4 HRP (1:2000 dilution, Invitrogen, A10654) in blocking solution at RT for 1 hr. After washing, the plates were developed with HRP substrate for 3 min. The reaction was stopped with 2N H₂SO₄ and the plate was read at 450 nm in a micro plate reader. The pharmacokinetics profiles are shown in FIG. 11 (Plasma concentration versus time profiles of 3 anti-C5a humanized antibodies N92, N97 and 16D10 after single I.V. injection at the dosage of 25 mg/kg.

FIG. 11 shows that both N97H and N92H had prolonged persistence in vivo up to 15 days after a single injection. Levels of 16D10 in Human C5/SCID/human FcRn mice decreased by day 3 following a single injection.

### Example 6: Impact of the Anti-C5a Antibodies on C5 Level In Vivo

SDS-PAGE and sandwich ELISA were used for detection of human C5 in FcRn/SCID mice expressing human C5 after hydrodynamic injection of human C5 cDNA plasmid.

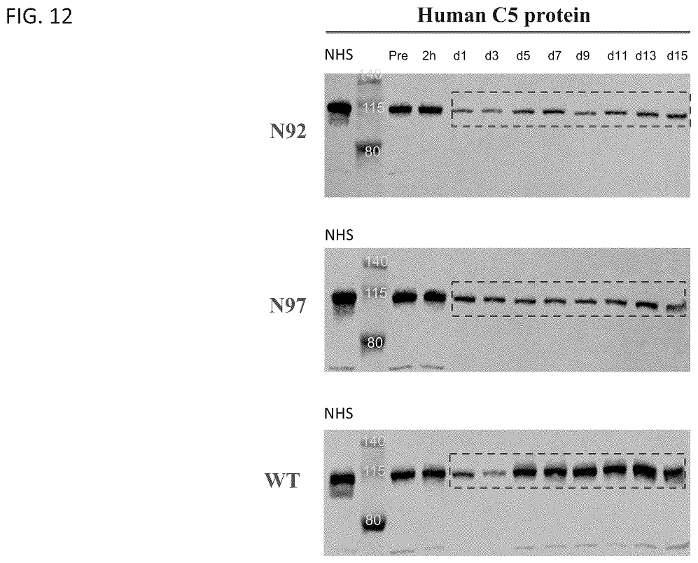

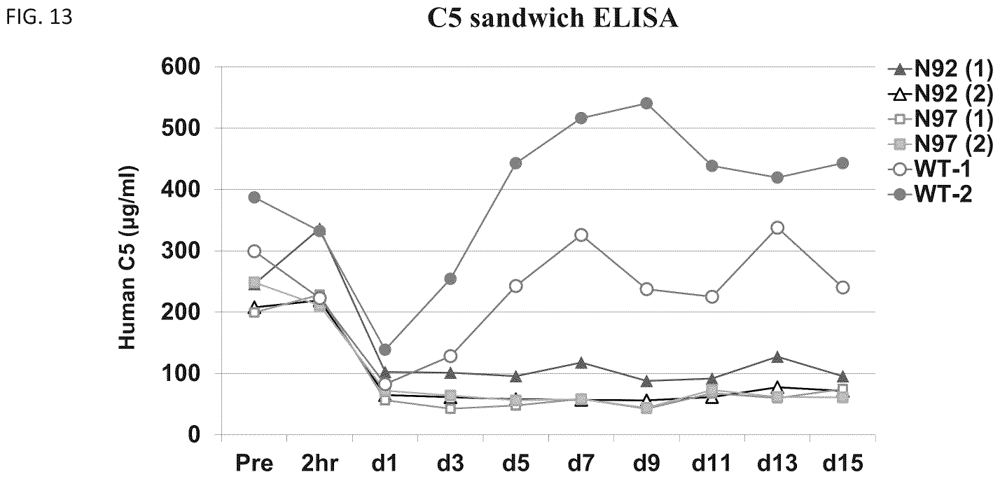

FIG. 12 shows the levels of human C5 protein in serum at various time points following injection of the anti-C5a antibodies N97H, N92H and 16D10 (denoted N97, N92 and WT in FIGS. 12 and 13, respectively). Both histidine mutation constructs lowered the level of C5. 16D10 initially lowered the level of C5 on day 1 and day 3 following injection, while the level of C5 recovered from day 5.

For sandwich ELISA, 96-well plates were coated with an anti-human C5 antibody (Quidel, A217) at a final concentration of 2 μg/mL in bicarbonate buffer at 37° C. for 1 hr. Following washes with PBS containing 0.05% Tween-20, the plates were incubated with diluted plasma samples in blocking solution at RT for 1 hr. The plates were then washed and incubated with biotinylated anti-human C5 mAb 9G6 in blocking solution at RT for 1 hour, washed again and incubated with avidin or streptavidin conjugated to horseradish peroxidase (BD pharmigen) in blocking solution at RT for 1 hr. After final washing, the plates were developed with HRP substrate for 3 min. The reaction was stopped with 2N H₂SO₄ and the plate was read at 450 nm in a micro plate reader. Results are shown in FIG. 13.

FIG. 13 shows that C5 levels after administration of N97H and N92H were lowered to approximately 25% of the pre-treatment levels, and the level of C5 following administration of 16D10 recovered from day 3 onwards, confirming the results from SDS-PAGE.

### Example 7: Level of C5 Required for Full Complement Activities

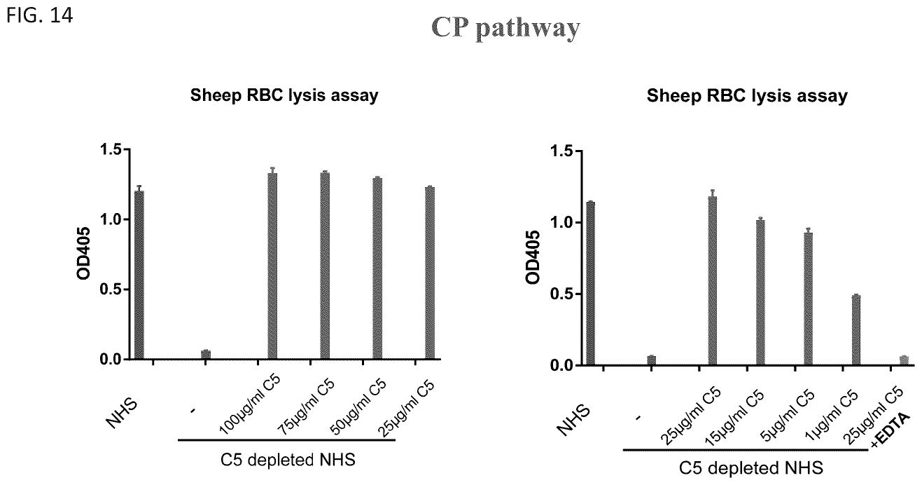

Sheep RBC lysis was performed to determine the level of C5 required for full activities of the classical pathway. A sample with only normal human serum incubation was used as a positive control for full complement activity. Samples without NHS or with EDTA added were used as negative lysis controls. As shown in FIG. 14, approximately 25% of C5 was sufficient to induce full complement activities through the classical pathway.

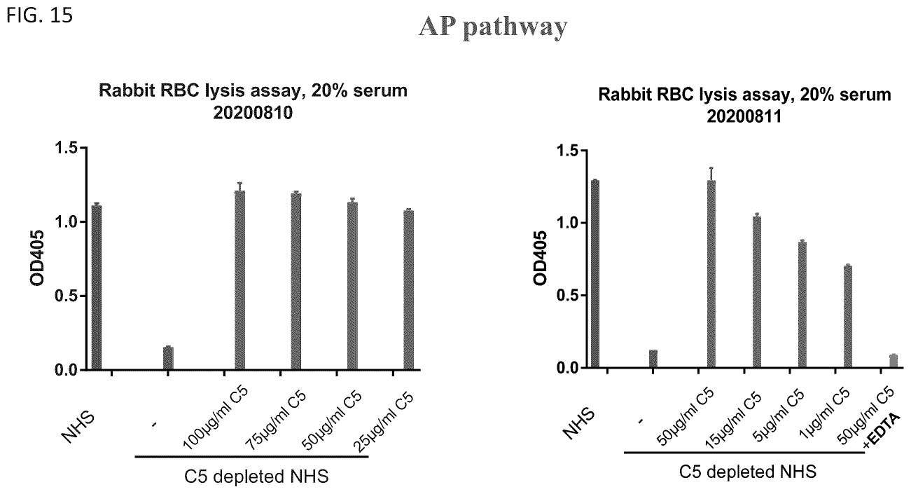

To assess the level of C5 required for alternative pathway complement-mediated hemolysis, a rabbit red blood cell lysis assay was performed. Rabbit RBCs (Rockland Immunochemicals Inc cat #R403-0100) (1×10⁷ cells per assay sample prepared in PBS, Complement Technology Inc.) were incubated at 37° C. for 30 min with 25% normal human serum (NHS, from Complement Technology Inc.) in gelatin veronal buffer (GVB2+EGTA, Sigma; total assay volume: 100 μL). A sample with only NHS incubation was used as a positive control for full complement activity. Samples without NHS or with EDTA added were used as negative lysis controls. Lysis reaction was stopped by addition of 40 mM EDTA dissolved in ice-cold PBS. The incubation mixtures were centrifuged for 5 min at 1500 rpm. The supernatant was collected and measured for OD405 nm. FIG. 15 shows that approximately 25% of C5 was sufficient to induce full complement activities through the alternative pathway. These results suggest that although the anti-C5a antibodies reduced C5 level in serum (Example 7), the remaining C5 was sufficient to induce full complement activities.

### Example 8: In Vivo Pharmacokinetic Evaluation of N92H Antibody in Cynomolgus Monkeys

In vivo pharmacokinetics evaluation of N92H antibody were tested in cynomolgus monkeys via a single intravenous dose of N92H antibody at 5, 30, and 100 mg/kg. Blood samples were collected at the following time points: predose (at most 2 days before the start of dosing), EOI (0-2 minutes prior to the end of infusion), and at 1, 6, 24, 72, 168, 336, 504, and 672 hours post end of infusion (EOI) on Day 1. In addition, blood samples were collected from the 5 mg/kg at 1728 (Week 11), 2184 (Week 14), and 2688 (Week 17) hours post EOI on Day 1.

The concentration of N92H antibody was determined using a qualified ELISA method with a lower limit of quantitation (LLOQ) of 37.3 ng/mL. This quantitative ELISA was based on a sandwich format, where all steps were performed at room temperature except for the coating where 4° C. was applied. A microwell plate was coated with a capture mouse anti-human IgG constant heavy chain 2 (CH2) antibody (Bio-Rad, MCA5748G) diluted in Coating Buffer. In the assay, standards, quality controls and samples were diluted at the minimal required dilution (MRD) in dilution buffer and distributed into pre-coated and blocked microwells. After incubation, the plate was washed to remove unbound material, prior to the well blocking step. Samples were then loaded in the wells and incubated at room temperature under agitation. After incubation, wells were washed prior to addition of a detection mouse anti-human IgG4 fragment crystallizable region (Fc)-horseradish peroxidase (HRP) conjugated antibody (Southern Biotech, 9200-05). The plate was washed, and a 3,3′,5,5′-Tetramethylbenzidine substrate solution (TMB) was added. After incubation, the reaction was stopped by addition of sulfuric acid stop solution. The absorbance was read at 450 nm using a reference wavelength at 650 nm. Color development is proportional to the amount of N92H antibody present in the wells.

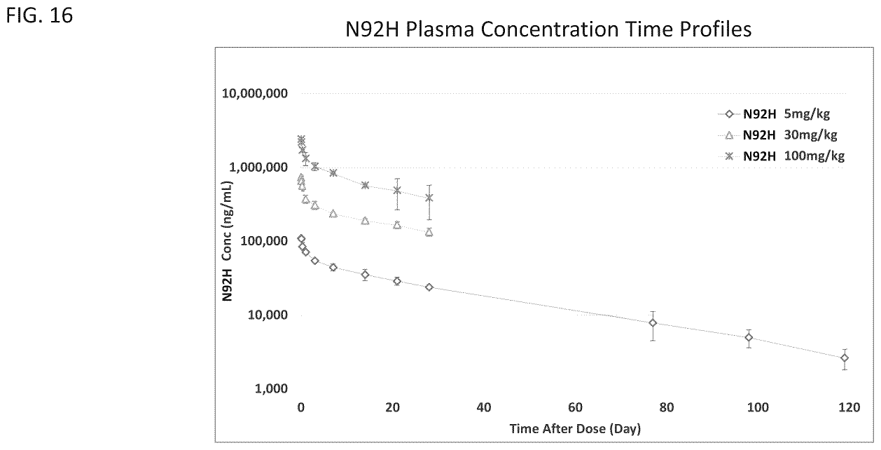

FIG. 16 shows that following a single 30-minute IV infusion of N92H antibody at 5, 30, and 100 mg/kg, plasma concentrations for all treatment groups were quantifiable throughout the 672-hour sampling period, including samples collected at Weeks 11, 14, and 17, at 5 mg/kg. The time to maximum plasma concentrations (tmax) of N92H antibody was observed at 0.5 (0 to 2 minutes prior the EOI) or 1.5 hours post SOI at 5 and 30 mg/kg and at 0.5 hours post SOI at 100 mg/kg. With extended sampling, the terminal phase was well characterized at 5 mg/kg with estimated values of 576 and 595 hours for terminal half-life (T½), 0.0922 and 0.109 mL/hr/kg for clearance (Cl), and 80.9 and 80.0 mL/kg for volume of distribution (Vss), for males and females, respectively.

### Example 9: In Vivo Pharmacodynamic Evaluation of N92H Antibody in Cynomolgus Monkeys

In vivo pharmacodynamic evaluation of N92H antibody were tested in cynomolgus monkeys via a single intravenous dose of N92H antibody at 5, 30, and 100 mg/kg. Blood samples were collected at the following time points: predose (at most 2 days before the start of dosing), EOI (0-2 minutes prior to the end of infusion), and at 1, 6, 24, 72, 168, 336, 504, and 672 hours post end of infusion (EOI) on Day 1. In addition, blood samples were collected from the 5 mg/kg at 1728 (Week 11), 2184 (Week 14), and 2688 (Week 17) hours post EOI on Day 1.

The pharmacodynamics of N92H antibody was determined using a procedure for the quasi-quantification of CD11b expression in Cynomolgus monkey whole blood by flow cytometry following ex-vivo C5a stimulation. Blood samples were collected in K2-EDTA tubes and transferred into in FACS tubes for immediate processing within 1 hour after initial collection time. Subsequently, recombinant cyno C5a (Sino Biological) was added to the samples for simulation followed by incubation with CD11b or isotype control antibodies (BD Biosciences, 557321 and 556650). The supernatant of red blood cells was obtained by mixing with lysis buffer (BD Biosciences, 555899). After fixing the cells using PFA (Alfa Aesar, J61899)/DPBS (Life Technologies, 14190136), the samples were submitted to FACS for the detection of CD11b signals.

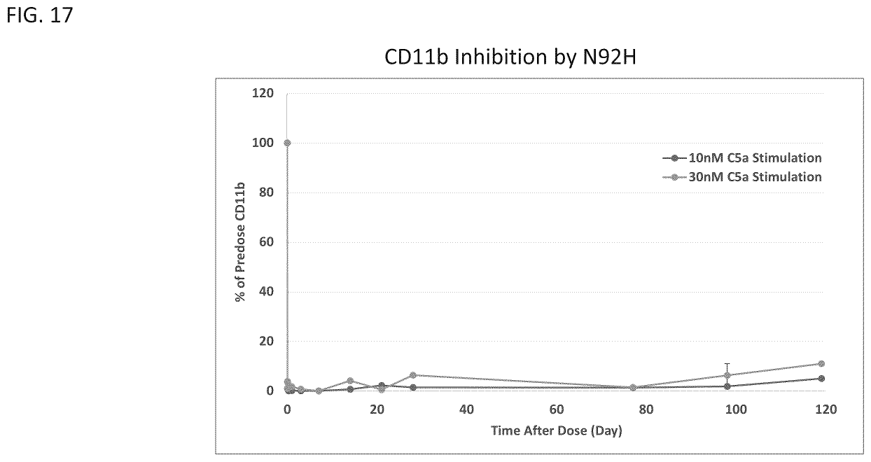

FIG. 17 shows that the C5a protein strongly increased the CD11b expression levels at the pre-dose, and its effects were inhibited in study samples in dosed animals throughout the course of the study. In animals receiving the low dose of N92H antibody (5 mg/kg), the stimulation with the C5a protein induced a strong increase of CD11b expression, both at 10 and 30 nM at the pre-dose. The stimulation of the C5a protein were inhibited and maintained up to Week 17. The inhibition of CD11b expression was relatively variable throughout all occasions but remained below 20%.

### Example 10: In Vivo Pharmacodynamic Evaluation of N92H Antibody in C5a-Induced Neutropenia Model in Cynomolgus Monkeys

C5a is one of the most potent pro-inflammatory mediators that induces expression of adhesion molecules and chemotactic migration of neutrophils. When C5a is generated locally in the bloodstream, C5aR-bearing neutrophils in the vicinity immediately upregulate adhesion molecules and adhere to the inner face of the blood vessel. If C5a is introduced systemically by intravenous injection, neutrophils adherence occurs immediately throughout the vasculature and, as a result, the number of neutrophils still flowing in the bloodstream drops transiently by a substantial amount. This phenomenon is termed neutropenia. We evaluated the in vivo effect of N92H antibody in the neutropenia model.

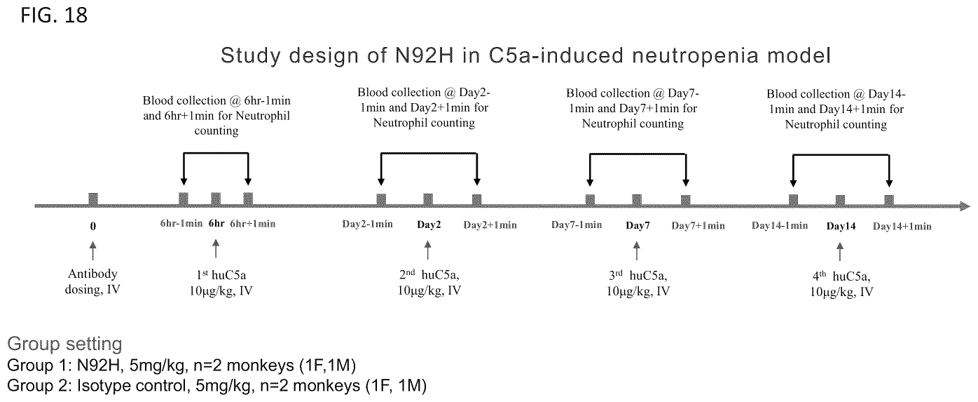

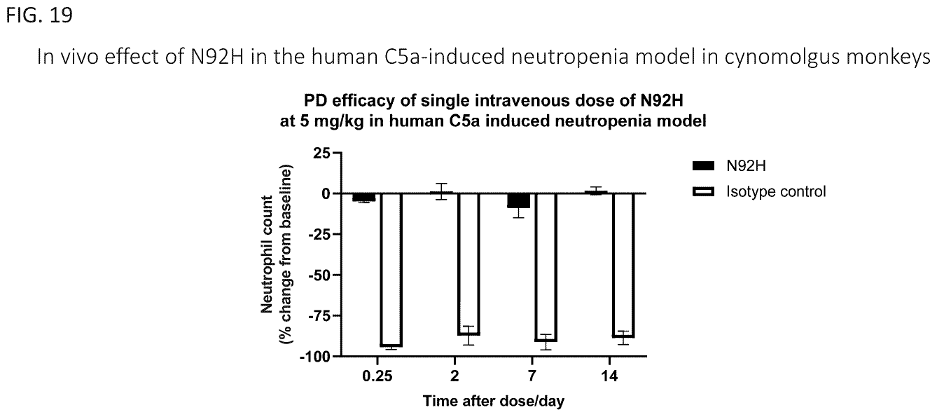

FIG. 18 describes the neutropenia model in monkey. Briefly, human C5a (10 μg/kg) was administrated intravenously at 6 hrs and subsequently at day 2, 7, and day 14 post N92H antibody administration. Blood samples were collected immediately at 1 minute (min) before and 1 min after each C5a injection and the neutrophil counts were analyzed by automated hematology analyzer (Sysmex XT-2000iV) within 2 hours. N92H antibody effect was calculated and expressed as the percent change of neutrophils in the blood samples 1 min before and 1 min after each C5a injection. The average reduction of neutrophils induced by C5a is about 90% at each of the C5a stimulation (data not shown). Data in in FIG. 19 show that monkeys pre-dosed with isotype control antibody has no impact on C5a-induced neutropenia. In contrast, monkeys pre-dosed with 5 mg/kg of N92H antibody showed more than 90% rescue of C5a-induced neutropenia. The rescue effect can last at least for 14 days.

SEQUENCE TABLE

| SEQ | | |
|---|---|---|
| ID NO | Notes | Amino Acid Sequences (mutations highlighted in yellow) |
| 1. | Anti-C5a mouse | QVQLQQSDAELVKPGASVKISCKVSGYTFTDHIIHWMNQRP |
| | mAb VH | EQGLEWIGYIYPRDGNTNYNENFKGKATLTADKSSSTAYMQ |
| | | LNSLTSEDSAVYFCARERNLEYFDYWGQGTTLTVSS |
| 2. | Anti-C5a mouse | DIVLTQSPASLAVSLGQRATISCKASQSVDYDGDNYMNWY |
| | mAb VL | QQKPGQPPKLLIYAASNLDSGIPARFSGSGSGTDFTLNIHPVE |
| | | EEDAATYYCQQSNEDPYTFGGGTKLEIK |
| 3. | 16D10 H-CDR1 | GYTFTDHIIH |
| | (Kabat) | |
| 4. | 16D10 H-CDR2 | YIYPREGWTNYNENFKG |
| | (Kabat) | |
| 5. | 16D10 H-CDR3 | ARERNLEYFDY |
| | (Kabat) | |
| 6. | 16D10 L-CDR1 | KASQSVDYEGFNYMN |
| | (Kabat) | |
| 7. | 16D10 L-CDR2 | AASNLDS |
| | (Kabat) | |
| 8. | 16D10 L-CDR3 | QQSNEDPYT |
| | (Kabat) | |
| 9. | 16D10 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 10. | 16D10 VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPYTFGGGTKVEIK |
| 11. | E54H H-CDR1 | GYTHTDHIIH |
| | (Kabat) | |
| 12. | E54H H-CDR2 | YIYPRHGWTNYNENFKG |
| | (Kabat) | |
| 13. | E54H H-CDR3 | ARERNLEYFDY |
| | (Kabat) | |
| 14. | E54H L- | KASQSVDYEGFNYMN |
| | CDR1(Kabat) | |
| 15. | E54H L-CDR2 | AASNLDS |
| | (Kabat) | |
| 16. | E54H L-CDR3 | QQSNEDPHT |
| | (Kabat) | |
| 17. | E54H VH | QVQLVQSGAEVKKPGSSVKVSCKASGYTHTDHIIHWMRQAP |
| | | GQGLEWMGYIYPRHGWTNYNENFKGRVTITADKSTSTAYM |
| | | ELSSLRSEDTAVYYCARERNLEYFDYWGQGTTVTVSS |
| 18. | E54H VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPHTFGGGTKVEIK |
| 19. | N92H H-CDR1 | GYTHTDHIIH |
| | (Kabat) | |
| 20. | N92H H-CDR2 | YIYPREGWTNYNENFKG |
| | (Kabat) | |
| 21. | N92H H-CDR3 | ARERNLEYFDY |
| | (Kabat) | |
| 22. | N92H L- | KASQSVDYEGFNYMN |
| | CDR1(Kabat) | |
| 23. | N92H L-CDR2 | AASNLDS |
| | (Kabat) | |
| 24. | N92H L-CDR3 | QQSHEDPHT |
| | (Kabat) | |
| 25. | N92H VH | QVQLVQSGAEVKKPGSSVKVSCKASGYTHTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 26. | N92H VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSHEDPHTFGGGTKVEIK |
| 27. | N97H H-CDR1 | GYTHTDHIIH |
| | (Kabat) | |
| 28. | N97H H-CDR2 | YIYPREGWTNYNENFKG |
| | (Kabat) | |
| 29. | N97H H-CDR3 | ARERHLEYFDY |
| | (Kabat) | |
| 30. | N97H L- | KASQSVDYEGFNYMN |
| | CDR1(Kabat) | |
| 31. | N97H L-CDR2 | AASNLDS |
| | (Kabat) | |
| 32. | N97H L-CDR3 | QQSNEDPHT |
| | (Kabat) | |
| 33. | N97H VH | QVQLVQSGAEVKKPGSSVKVSCKASGYTHTDHIIHWMRQAP |
| | | GQGLEWMGYIYPREGWTNYNENFKGRVTITADKSTSTAYM |
| | | ELSSLRSEDTAVYYCARERHLEYFDYWGQGTTVTVSS |
| 34. | N97H VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPHTFGGGTKVEIK |
| 35. | 16D10 HC | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVLHEALHAHYTQKSLSLSLGK |
| 36. | 16D10 LC | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPYTFGGGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 37. | E54H HC | QVQLVQSGAEVKKPGSSVKVSCKASGYTHTDHIIHWMRQAP |
| | | GQGLEWMGYIYPRHGWTNYNENFKGRVTITADKSTSTAYM |
| | | ELSSLRSEDTAVYYCARERNLEYFDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVLHEALHAHYTQKSLSLSLGK |
| 38. | E54H LC | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPHTFGGGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 39. | N92H HC | QVQLVQSGAEVKKPGSSVKVSCKASGYTHTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVLHEALHAHYTQKSLSLSLGK |
| 40. | N92H LC | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSHEDPHTFGGGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 41 | N97H HC | QVQLVQSGAEVKKPGSSVKVSCKASGYTHTDHIIHWMRQAP |
| | | GQGLEWMGYIYPREGWTNYNENFKGRVTITADKSTSTAYM |
| | | ELSSLRSEDTAVYYCARERHLEYFDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVLHEALHAHYTQKSLSLSLGK |
| 42. | N97H LC | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSNEDPHTFGGGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 43. | WT IgG4 | ASTKGPSVFPLAPCSRSTSESTAALGCLVKDYFPEPVTVSWN |
| | gamma Fc | SGALTSGVHTFPAVLQSSGLYSLSSVVTVPSSSLGTKTYTCN |
| | | VDHKPSNTKVDKRVESKYGPPCPSCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| 44 | LA mutation | ASTKGPSVFPLAPCSRSTSESTAALGCLVKDYFPEPVTVSWN |
| | IgG4 Fc | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHAHYTQKSLSLSLGK |
| 45. | Human C5a | TLQKKIEEIAAKYKHSVVKKCCYDGACVNNDETCEQRAARI |
| | | SLGPRCIKAFTECCVVASQLRANISHKDMQLGR |
| 46. | 16D10 | CAAGTGCAGCTGGTGCAGTCCGGAGCTGAAGTGAAGAAG |
| | | CCCGGCAGCAGCGTGAAGGTGAGCTGTAAGGCCTCCGGCT |
| | | ACACATTCACTGACCACATCATCCACTGGATGAGGCAAGC |
| | | CCCCGGCCAAGGACTGGAGTGGATGGGCTACATCTACCCA |
| | | AGGGAGGGATGGACTAACTACAACGAGAACTTCAAGGGA |
| | | AGGGTGACAATCACAGCCGACAAGTCCACAAGCACAGCC |
| | | TACATGGAACTCAGCTCTCTGAGAAGCGAGGATACTGCCG |
| | | TGTACTACTGCGCTAGGGAGAGGAATCTGGAGTACTTCGA |
| | | CTACTGGGGCCAAGGCACAACAGTGACTGTGAGCAGCGC |
| | | TAGCACCAAGGGACCTAGCGTGTTTCCTCTGGCCCCTTGT |
| | | AGCAGAAGCACCAGCGAAAGCACAGCCGCTCTGGGCTGT |
| | | CTGGTGAAAGACTACTTTCCCGAGCCCGTGACCGTGTCTT |
| | | GGAACAGCGGAGCCCTGACCAGCGGAGTGCACACATTTC |
| | | CAGCCGTGCTCCAGAGCAGCGGACTGTATAGCCTGAGCAG |
| | | CGTGGTGACCGTGCCTTCTTCTAGCCTGGGCACCAAGACC |
| | | TACACCTGCAACGTGGACCACAAGCCCAGCAACACCAAG |
| | | GTGGACAAGCGGGTGGAGAGCAAATACGGCCCTCCTTGC |
| | | CCTCCTTGCCCAGCTCCAGAGTTTCTGGGAGGACCTAGCG |
| | | TGTTCCTGTTCCCTCCCAAGCCCAAGGACACCCTGATGAT |
| | | CAGCCGGACCCCAGAAGTCACCTGCGTGGTGGTGGACGTG |
| | | TCTCAGGAAGACCCCGAGGTGCAGTTCAACTGGTACGTGG |
| | | ACGGCGTGGAGGTGCACAACGCTAAGACCAAGCCCAGGG |
| | | AGGAGCAGTTCAACAGCACCTACAGGGTGGTGTCCGTGCT |
| | | GACAGTGCTGCATCAGGATTGGCTGAACGGCAAGGAGTA |
| | | CAAGTGCAAGGTGTCCAACAAGGGCCTGCCTAGCAGCATC |
| | | GAGAAGACCATCAGCAAGGCCAAGGGCCAGCCTAGAGAG |
| | | CCTCAGGTGTACACACTGCCCCCTTCTCAGGAGGAGATGA |
| | | CCAAGAACCAGGTGTCCCTGACTTGCCTCGTGAAGGGCTT |
| | | CTACCCCAGCGATATTGCCGTGGAGTGGGAGTCTAACGGC |
| | | CAGCCCGAGAACAACTACAAGACCACCCCTCCCGTGCTGG |
| | | ATAGCGACGGCTCTTTCTTCCTGTACAGCCGGCTGACAGT |
| | | GGACAAAAGTCGCTGGCAGGAGGGCAACGTGTTCAGTTG |
| | | CAGCGTGCTGCACGAGGCTCTGCACGCCCACTATACCCAG |
| | | AAGAGCCTGAGCCTGAGCCTGGGAAAG |
| 47 | 16D10 VL | GATATTGTGCTGACACAGTCTCCAGCTTCTCTGGCAGTGTC |
| | | TCCAGGACAGAGAGCTACAATTACTTGTAAGGCCTCTCAG |
| | | TCCGTGGATTACGAGGGCTTCAACTACATGAACTGGTATC |
| | | AGCAGAAACCAGGACAGCCTCCTAAACTGCTGATCTACGC |
| | | CGCTTCTAATCTGGATTCCGGAGTGCCAGCAAGATTTTCC |
| | | GGCTCCGGCTCTGGCACCGATTTTACCCTGACCATCAATC |
| | | CAGTGGAAGCCGAGGATACCGCTAACTACTATTGCCAGCA |
| | | GTCTAACGAGGACCCTTATACATTTGGCGGCGGAACAAAG |
| | | GTGGAGATTAAGCGTACGGTGGCCGCTCCTAGCGTGTTCA |
| | | TCTTCCCTCCCAGCGACGAGCAGCTGAAAAGCGGAACAGC |
| | | CAGCGTCGTCTGCCTGCTGAATAACTTCTACCCCAGAGAG |
| | | GCCAAAGTCCAGTGGAAAGTGGACAACGCCCTCCAGAGC |
| | | GGAAACTCTCAGGAGAGCGTGACCGAGCAGGACAGCAAG |
| | | GACAGCACCTACAGCCTGAGCAGCACACTGACCCTGAGC |
| | | AAGGCCGACTACGAGAAGCACAAGGTGTACGCTTGCGAG |
| | | GTCACACACCAGGGACTGTCTAGCCCAGTGACCAAGAGCT |
| | | TCAACCGCGGCGAGTGT |
| 48. | E54H VH | CAAGTGCAGCTGGTGCAGTCCGGAGCTGAAGTGAAGAAG |
| | | CCCGGCAGCAGCGTGAAGGTGAGCTGTAAGGCCTCCGGCT |
| | | ACACACACACTGACCACATCATCCACTGGATGAGGCAAGC |
| | | CCCCGGCCAAGGACTGGAGTGGATGGGCTACATCTACCCA |
| | | AGGCACGGATGGACTAACTACAACGAGAACTTCAAGGGA |
| | | AGGGTGACAATCACAGCCGACAAGTCCACAAGCACAGCC |
| | | TACATGGAACTCAGCTCTCTGAGAAGCGAGGATACTGCCG |
| | | TGTACTACTGCGCTAGGGAGAGGAATCTGGAGTACTTCGA |
| | | CTACTGGGGCCAAGGCACAACAGTGACTGTGAGCAGCGC |
| | | TAGCACCAAGGGACCTAGCGTGTTTCCTCTGGCCCCTTGT |
| | | AGCAGAAGCACCAGCGAAAGCACAGCCGCTCTGGGCTGT |
| | | CTGGTGAAAGACTACTTTCCCGAGCCCGTGACCGTGTCTT |
| | | GGAACAGCGGAGCCCTGACCAGCGGAGTGCACACATTTC |
| | | CAGCCGTGCTCCAGAGCAGCGGACTGTATAGCCTGAGCAG |
| | | CGTGGTGACCGTGCCTTCTTCTAGCCTGGGCACCAAGACC |
| | | TACACCTGCAACGTGGACCACAAGCCCAGCAACACCAAG |
| | | GTGGACAAGCGGGTGGAGAGCAAATACGGCCCTCCTTGC |
| | | CCTCCTTGCCCAGCTCCAGAGTTTCTGGGAGGACCTAGCG |
| | | TGTTCCTGTTCCCTCCCAAGCCCAAGGACACCCTGATGAT |
| | | CAGCCGGACCCCAGAAGTCACCTGCGTGGTGGTGGACGTG |
| | | TCTCAGGAAGACCCCGAGGTGCAGTTCAACTGGTACGTGG |
| | | ACGGCGTGGAGGTGCACAACGCTAAGACCAAGCCCAGGG |
| | | AGGAGCAGTTCAACAGCACCTACAGGGTGGTGTCCGTGCT |
| | | GACAGTGCTGCATCAGGATTGGCTGAACGGCAAGGAGTA |
| | | CAAGTGCAAGGTGTCCAACAAGGGCCTGCCTAGCAGCATC |
| | | GAGAAGACCATCAGCAAGGCCAAGGGCCAGCCTAGAGAG |
| | | CCTCAGGTGTACACACTGCCCCCTTCTCAGGAGGAGATGA |
| | | CCAAGAACCAGGTGTCCCTGACTTGCCTCGTGAAGGGCTT |
| | | CTACCCCAGCGATATTGCCGTGGAGTGGGAGTCTAACGGC |
| | | CAGCCCGAGAACAACTACAAGACCACCCCTCCCGTGCTGG |
| | | ATAGCGACGGCTCTTTCTTCCTGTACAGCCGGCTGACAGT |
| | | GGACAAAAGTCGCTGGCAGGAGGGCAACGTGTTCAGTTG |
| | | CAGCGTGCTGCACGAGGCTCTGCACGCCCACTATACCCAG |
| | | AAGAGCCTGAGCCTGAGCCTGGGAAAG |
| 49. | E54H VL | GATATTGTGCTGACACAGTCTCCAGCTTCTCTGGCAGTGTC |
| | | TCCAGGACAGAGAGCTACAATTACTTGTAAGGCCTCTCAG |
| | | TCCGTGGATTACGAGGGCTTCAACTACATGAACTGGTATC |
| | | AGCAGAAACCAGGACAGCCTCCTAAACTGCTGATCTACGC |
| | | CGCTTCTAATCTGGATTCCGGAGTGCCAGCAAGATTTTCC |
| | | GGCTCCGGCTCTGGCACCGATTTTACCCTGACCATCAATC |
| | | CAGTGGAAGCCGAGGATACCGCTAACTACTATTGCCAGCA |
| | | GTCTAACGAGGACCCTCACACATTTGGCGGCGGAACAAA |
| | | GGTGGAGATTAAGCGTACGGTGGCCGCTCCTAGCGTGTTC |
| | | ATCTTCCCTCCCAGCGACGAGCAGCTGAAAAGCGGAACA |
| | | GCCAGCGTCGTCTGCCTGCTGAATAACTTCTACCCCAGAG |
| | | AGGCCAAAGTCCAGTGGAAAGTGGACAACGCCCTCCAGA |
| | | GCGGAAACTCTCAGGAGAGCGTGACCGAGCAGGACAGCA |
| | | AGGACAGCACCTACAGCCTGAGCAGCACACTGACCCTGA |
| | | GCAAGGCCGACTACGAGAAGCACAAGGTGTACGCTTGCG |
| | | AGGTCACACACCAGGGACTGTCTAGCCCAGTGACCAAGA |
| | | GCTTCAACCGCGGCGAGTGT |
| 50. | N97H VH | CAAGTGCAGCTGGTGCAGTCCGGAGCTGAAGTGAAGAAG |
| | | CCCGGCAGCAGCGTGAAGGTGAGCTGTAAGGCCTCCGGCT |
| | | ACACACACACTGACCACATCATCCACTGGATGAGGCAAGC |
| | | CCCCGGCCAAGGACTGGAGTGGATGGGCTACATCTACCCA |
| | | AGGGAGGGATGGACTAACTACAACGAGAACTTCAAGGGA |
| | | AGGGTGACAATCACAGCCGACAAGTCCACAAGCACAGCC |
| | | TACATGGAACTCAGCTCTCTGAGAAGCGAGGATACTGCCG |
| | | TGTACTACTGCGCTAGGGAGAGGCACCTGGAGTACTTCGA |
| | | CTACTGGGGCCAAGGCACAACAGTGACTGTGAGCAGCGC |
| | | TAGCACCAAGGGACCTAGCGTGTTTCCTCTGGCCCCTTGT |
| | | AGCAGAAGCACCAGCGAAAGCACAGCCGCTCTGGGCTGT |
| | | CTGGTGAAAGACTACTTTCCCGAGCCCGTGACCGTGTCTT |
| | | GGAACAGCGGAGCCCTGACCAGCGGAGTGCACACATTTC |
| | | CAGCCGTGCTCCAGAGCAGCGGACTGTATAGCCTGAGCAG |
| | | CGTGGTGACCGTGCCTTCTTCTAGCCTGGGCACCAAGACC |
| | | TACACCTGCAACGTGGACCACAAGCCCAGCAACACCAAG |
| | | GTGGACAAGCGGGTGGAGAGCAAATACGGCCCTCCTTGC |
| | | CCTCCTTGCCCAGCTCCAGAGTTTCTGGGAGGACCTAGCG |
| | | TGTTCCTGTTCCCTCCCAAGCCCAAGGACACCCTGATGAT |
| | | CAGCCGGACCCCAGAAGTCACCTGCGTGGTGGTGGACGTG |
| | | TCTCAGGAAGACCCCGAGGTGCAGTTCAACTGGTACGTGG |
| | | ACGGCGTGGAGGTGCACAACGCTAAGACCAAGCCCAGGG |
| | | AGGAGCAGTTCAACAGCACCTACAGGGTGGTGTCCGTGCT |
| | | GACAGTGCTGCATCAGGATTGGCTGAACGGCAAGGAGTA |
| | | CAAGTGCAAGGTGTCCAACAAGGGCCTGCCTAGCAGCATC |
| | | GAGAAGACCATCAGCAAGGCCAAGGGCCAGCCTAGAGAG |
| | | CCTCAGGTGTACACACTGCCCCCTTCTCAGGAGGAGATGA |
| | | CCAAGAACCAGGTGTCCCTGACTTGCCTCGTGAAGGGCTT |
| | | CTACCCCAGCGATATTGCCGTGGAGTGGGAGTCTAACGGC |
| | | CAGCCCGAGAACAACTACAAGACCACCCCTCCCGTGCTGG |
| | | ATAGCGACGGCTCTTTCTTCCTGTACAGCCGGCTGACAGT |
| | | GGACAAAAGTCGCTGGCAGGAGGGCAACGTGTTCAGTTG |
| | | CAGCGTGCTGCACGAGGCTCTGCACGCCCACTATACCCAG |
| | | AAGAGCCTGAGCCTGAGCCTGGGAAAG |
| 51. | N97H VL | GATATTGTGCTGACACAGTCTCCAGCTTCTCTGGCAGTGTC |
| | | TCCAGGACAGAGAGCTACAATTACTTGTAAGGCCTCTCAG |
| | | TCCGTGGATTACGAGGGCTTCAACTACATGAACTGGTATC |
| | | AGCAGAAACCAGGACAGCCTCCTAAACTGCTGATCTACGC |
| | | CGCTTCTAATCTGGATTCCGGAGTGCCAGCAAGATTTTCC |
| | | GGCTCCGGCTCTGGCACCGATTTTACCCTGACCATCAATC |
| | | CAGTGGAAGCCGAGGATACCGCTAACTACTATTGCCAGCA |
| | | GTCTAACGAGGACCCTCACACATTTGGCGGCGGAACAAA |
| | | GGTGGAGATTAAGCGTACGGTGGCCGCTCCTAGCGTGTTC |
| | | ATCTTCCCTCCCAGCGACGAGCAGCTGAAAAGCGGAACA |
| | | GCCAGCGTCGTCTGCCTGCTGAATAACTTCTACCCCAGAG |
| | | AGGCCAAAGTCCAGTGGAAAGTGGACAACGCCCTCCAGA |
| | | GCGGAAACTCTCAGGAGAGCGTGACCGAGCAGGACAGCA |
| | | AGGACAGCACCTACAGCCTGAGCAGCACACTGACCCTGA |
| | | GCAAGGCCGACTACGAGAAGCACAAGGTGTACGCTTGCG |
| | | AGGTCACACACCAGGGACTGTCTAGCCCAGTGACCAAGA |
| | | GCTTCAACCGCGGCGAGTGT |
| 52. | N92H VH | CAAGTGCAGCTGGTGCAGTCCGGAGCTGAAGTGAAGAAG |
| | | CCCGGCAGCAGCGTGAAGGTGAGCTGTAAGGCCTCCGGCT |
| | | ACACACACACTGACCACATCATCCACTGGATGAGGCAAGC |
| | | CCCCGGCCAAGGACTGGAGTGGATGGGCTACATCTACCCA |
| | | AGGGAGGGATGGACTAACTACAACGAGAACTTCAAGGGA |
| | | AGGGTGACAATCACAGCCGACAAGTCCACAAGCACAGCC |
| | | TACATGGAACTCAGCTCTCTGAGAAGCGAGGATACTGCCG |
| | | TGTACTACTGCGCTAGGGAGAGGAATCTGGAGTACTTCGA |
| | | CTACTGGGGCCAAGGCACAACAGTGACTGTGAGCAGCGC |
| | | TAGCACCAAGGGACCTAGCGTGTTTCCTCTGGCCCCTTGT |
| | | AGCAGAAGCACCAGCGAAAGCACAGCCGCTCTGGGCTGT |
| | | CTGGTGAAAGACTACTTTCCCGAGCCCGTGACCGTGTCTT |
| | | GGAACAGCGGAGCCCTGACCAGCGGAGTGCACACATTTC |
| | | CAGCCGTGCTCCAGAGCAGCGGACTGTATAGCCTGAGCAG |
| | | CGTGGTGACCGTGCCTTCTTCTAGCCTGGGCACCAAGACC |
| | | TACACCTGCAACGTGGACCACAAGCCCAGCAACACCAAG |
| | | GTGGACAAGCGGGTGGAGAGCAAATACGGCCCTCCTTGC |
| | | CCTCCTTGCCCAGCTCCAGAGTTTCTGGGAGGACCTAGCG |
| | | TGTTCCTGTTCCCTCCCAAGCCCAAGGACACCCTGATGAT |
| | | CAGCCGGACCCCAGAAGTCACCTGCGTGGTGGTGGACGTG |
| | | TCTCAGGAAGACCCCGAGGTGCAGTTCAACTGGTACGTGG |
| | | ACGGCGTGGAGGTGCACAACGCTAAGACCAAGCCCAGGG |
| | | AGGAGCAGTTCAACAGCACCTACAGGGTGGTGTCCGTGCT |
| | | GACAGTGCTGCATCAGGATTGGCTGAACGGCAAGGAGTA |
| | | CAAGTGCAAGGTGTCCAACAAGGGCCTGCCTAGCAGCATC |
| | | GAGAAGACCATCAGCAAGGCCAAGGGCCAGCCTAGAGAG |
| | | CCTCAGGTGTACACACTGCCCCCTTCTCAGGAGGAGATGA |
| | | CCAAGAACCAGGTGTCCCTGACTTGCCTCGTGAAGGGCTT |
| | | CTACCCCAGCGATATTGCCGTGGAGTGGGAGTCTAACGGC |
| | | CAGCCCGAGAACAACTACAAGACCACCCCTCCCGTGCTGG |
| | | ATAGCGACGGCTCTTTCTTCCTGTACAGCCGGCTGACAGT |
| | | GGACAAAAGTCGCTGGCAGGAGGGCAACGTGTTCAGTTG |
| | | CAGCGTGCTGCACGAGGCTCTGCACGCCCACTATACCCAG |
| | | AAGAGCCTGAGCCTGAGCCTGGGAAAG |
| 53. | N92H VL | GATATTGTGCTGACACAGTCTCCAGCTTCTCTGGCAGTGTC |
| | | TCCAGGACAGAGAGCTACAATTACTTGTAAGGCCTCTCAG |
| | | TCCGTGGATTACGAGGGCTTCAACTACATGAACTGGTATC |
| | | AGCAGAAACCAGGACAGCCTCCTAAACTGCTGATCTACGC |
| | | CGCTTCTAATCTGGATTCCGGAGTGCCAGCAAGATTTTCC |
| | | GGCTCCGGCTCTGGCACCGATTTTACCCTGACCATCAATC |
| | | CAGTGGAAGCCGAGGATACCGCTAACTACTATTGCCAGCA |
| | | GTCTCACGAGGACCCTCACACATTTGGCGGCGGAACAAAG |
| | | GTGGAGATTAAGCGTACGGTGGCCGCTCCTAGCGTGTTCA |
| | | TCTTCCCTCCCAGCGACGAGCAGCTGAAAAGCGGAACAGC |
| | | CAGCGTCGTCTGCCTGCTGAATAACTTCTACCCCAGAGAG |
| | | GCCAAAGTCCAGTGGAAAGTGGACAACGCCCTCCAGAGC |
| | | GGAAACTCTCAGGAGAGCGTGACCGAGCAGGACAGCAAG |
| | | GACAGCACCTACAGCCTGAGCAGCACACTGACCCTGAGC |
| | | AAGGCCGACTACGAGAAGCACAAGGTGTACGCTTGCGAG |
| | | GTCACACACCAGGGACTGTCTAGCCCAGTGACCAAGAGCT |
| | | TCAACCGCGGCGAGTGT |
| 54. | 2H8E10 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 55. | 2H8E10 VL | DIVLTQSPASLAVSPGQRATITCKASQSVDYEGDNYMNWYQ |
| | | QKPGQPPKLLIYAASNLDSGVPARFSGSGSGTDFTLTINPVEA |
| | | EDTANYYCQQSNEDPHTFGGGTKVEIK |
| 56. | 16F10 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 57. | 16F10 VL | DIVLTQSPASLAVSPGQRATITCKASQSVDFEGFNYMNWYQ |
| | | QKPGQPPKLLIYAASNLDSGVPARFSGSGSGTDFTLTINPVEA |
| | | EDTANYYCQQSVEDPHTFGGGTKVEIK |
| 58. | 13B7 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 59. | 13B7 VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSVEDPHTFGGGTKVEIK |
| 60. | 5G11 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 61. | 5G11 VL | DIVLTQSPASLAVSPGQRATITCKASQSVDYEGFNYMNWYQ |
| | | QKPGQPPKLLIYAASNLDSGVPARFSGSGSGTDFTLTINPVEA |
| | | EDTANYYCQQSLEDPHTFGGGTKVEIK |
| 62. | 18F5 VH | QVQLVQSGAEVKKPGSSVKVSCKASGYTFTDHIIHWMRQAP |
| | | GQGLEWMGYIYPREGFTNYNENFKGRVTITADKSTSTAYME |
| | | LSSLRSEDTAVYYCARERNLEYFDYWGQGTTVTVSS |
| 63. | 18F5 VL | DIVLTQSPASLAVSPGQRATITCKASQSVDFEGFNYMNWYQ |
| | | QKPGQPPKLLIYAASNLDSGVPARFSGSGSGTDFTLTINPVEA |
| | | EDTANYYCQQSVEDPHTFGGGTKVEIK |
| 64. | 13B9 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 65. | 13B9 VL | DIVLTQSPASLAVSPGQRATITCKASQSVDFEGFNYMNWYQ |
| | | QKPGQPPKLLIYAASNLDSGVPARFSGSGSGTDFTLTINPVEA |
| | | EDTANYYCQQSNEDPHTFGGGTKVEIK |
| 66. | 8C1 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 67. | 8C1 VL | DIVLTQSPASLAVSPGQRATITCKASQSVDFEGFNYMNWYQ |
| | | QKPGQPPKLLIYAASNLDSGVPARFSGSGSGTDFTLTINPVEA |
| | | EDTANYYCQQSLEDPHTFGGGTKVEIK |
| 68. | 17D2 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 69. | 17D2 VL | DIVLTQSPASLAVSPGQRATITCKASQSVDFEGDNYMNWYQ |
| | | QKPGQPPKLLIYATSNLDSGVPARFSGSGSGTDFTLTINPVEA |
| | | EDTANYYCQQSVEDPHTFGGGTKVEIK |
| 70. | 10F5 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 71. | 10F5 VL | DIVLTQSPASLAVSPGQRATITCKASQSVDYEGDNYMNWYQ |
| | | QKPGQPPKLLIYATSNLDSGVPARFSGSGSGTDFTLTINPVEA |
| | | EDTANYYCQQSLEDPHTFGGGTKVEIK |
| 72. | 16C2 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 73. | 16C2 VL | DIVLTQSPASLAVSPGQRATITCKASQSVDYEGFNYMNWYQ |
| | | QKPGQPPKLLIYATSNLDSGVPARFSGSGSGTDFTLTINPVEA |
| | | EDTANYYCQQSVEDPHTFGGGTKVEIK |
| 74. | 19D2 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 75. | 19D2 VL | DIVLTQSPASLAVSPGQRATITCKASQSVDFEGDNYMNWYQ |
| | | QKPGQPPKLLIYATSNLDSGVPARFSGSGSGTDFTLTINPVEA |
| | | EDTANYYCQQSLEDPHTFGGGTKVEIK |
| 76. | 8H8 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 77. | 8H8 VL | DIVLTQSPASLAVSPGQRATITCKASQSVDYEGFNYMNWYQ |
| | | QKPGQPPKLLIYATSNLDSGVPARFSGSGSGTDFTLTINPVEA |
| | | EDTANYYCQQSLEDPHTFGGGTKVEIK |
| 78. | 11A11 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 79. | 11A11 VL | DIVLTQSPASLAVSPGQRATITCKASQSVDFEGFNYMNWYQ |
| | | QKPGQPPKLLIYATSNLDSGVPARFSGSGSGTDFTLTINPVEA |
| | | EDTANYYCQQSVEDPHTFGGGTKVEIK |
| 80. | 18D3 VH | QVQLVQSGAEVKKPGSSVKVSCKASGYTFTDHIIHWMRQAP |
| | | GQGLEWMGYIYPREGWTNYNENFKGRVTITADKSTSTAYM |
| | | ELSSLRSEDTAVYYCARERNLEYFDYWGQGTTVTVSS |
| 81. | 18D3 VL | DIVLTQSPASLAVSPGQRATITCKASQSVDYEGDNYMNWYQ |
| | | QKPGQPPKLLIYATSNLDSGVPARFSGSGSGTDFTLTINPVEA |
| | | EDTANYYCQQSVEDPHTFGGGTKVEIK |
| 82. | NM2 VH for | QVQLVQSGAEVKKPGSSVKVSCKASGYTFTDHIIHWMRQAP |
| | mutant library | GQGLEWMGYIYPREGNTNYNENFKGRVTITADKSTSTAYME |
| | screening | LSSLRSEDTAVYYCARERNLEYFDYWGQGTTVTVSS |
| 83. | NM2 VL for | DIVLTQSPASLAVSPGQRATITCKASQSVDYEGDNYMNWYQ |
| | mutant library | QKPGQPPKLLIYAASNLDSGVPARFSGSGSGTDFTLTINPVEA |
| | screening | EDTANYYCQQSNEDPHTFGGGTKVEIK |
| 84. | CDR grafting | QVQLVQSGAEVKKPGSSVKVSCKASGYTFTDHIIHWMRQAP |
| | VH for affinity | GQGLEWMGYIYPRDGNTNYNENFKGRVTITADKSTSTAYM |
| | maturation | ELSSLRSEDTAVYYCARERNLEYFDYWGQGTTVTVSS |
| 85. | CDR grafting | DIVLTQSPASLAVSPGQRATITCKASQSVDYDGDNYMNWYQ |
| | VL for affinity | QKPGQPPKLLIYAASNLDSGVPARFSGSGSGTDFTLTINPVEA |
| | maturation | EDTANYYCQQSNEDPYTFGGGTKVEIK |

What is claimed is:

1. An isolated, humanized antibody that specifically binds to human C5a and C5, wherein the antibody comprises a heavy chain variable domain (VH) and a light chain variable domain (VL), wherein VH comprises I48M, D54E, and N56W mutations, wherein VL comprises D28E and D30F mutations, wherein the VH mutation is in reference to SEQ ID NO:1 under the Kabat numbering system, and wherein the VL mutation is in reference to SEQ ID NO:2 under the Kabat numbering system, and wherein the antibody comprises:
i) a heavy chain CDR1 (“H-CDR1”) comprising the amino acid sequence of SEQ ID NO:3 or a variant thereof comprising one, two, or three amino acid substitutions;
ii) a heavy chain CDR2 (“H-CDR2”) comprising the amino acid sequence of SEQ ID NO:4 or a variant thereof comprising one, two, or three amino acid substitutions;
iii) a heavy chain CDR3 (“H-CDR3”) comprising the amino acid sequence of SEQ ID NO:5 or a variant thereof comprising one, two, or three amino acid substitutions;
iv) a light chain CDR1 (“L-CDR1”) comprising the amino acid sequence of SEQ ID NO:6 or a variant thereof comprising one, two, or three amino acid substitutions;
v) a light chain CDR2 (“L-CDR2”) comprising the amino acid sequence of SEQ ID NO:7 or a variant thereof comprising one, two, or three amino acid substitutions; and
vi) a light chain CDR3 (L-CDR3”) comprising the amino acid sequence of SEQ ID NO:8 or a variant thereof comprising one, two, or three amino acid substitutions.

2. The antibody of claim 1, wherein the antibody further comprises an F29H mutation in VH and a Y96H mutation in the VL, wherein the VH mutation is in reference to SEQ ID NO:1 under the Kabat numbering system, and wherein the VL mutation is in reference to SEQ ID NO:2 under the Kabat numbering system.

3. The antibody of claim 2, wherein the antibody further comprises a substitution in the VH or VL.

4. The antibody of claim 3, wherein the mutation is selected from the group consisting of: E54H of VH, N97H of VH, and N92H of VL, wherein the VH mutation is in reference to SEQ ID NO:1 under the Kabat numbering system, and wherein the VL mutation is in reference to SEQ ID NO:2 under the Kabat numbering system.

5. The antibody of claim 1, comprising:
i) a VH comprising the amino acid sequence SEQ ID NO:9 or a variant thereof that is at least about 85% identical to SEQ ID NO:9; and
ii) a V_{L} comprising the amino acid sequence of SEQ ID NO:10 or a variant thereof that is at least about 85% identical to SEQ ID NO:10.

6. The antibody of any one of claims 1-4, wherein the antibody comprises:
i) H-CDR1 comprising the amino acid sequence of SEQ ID NO:11;
ii) H-CDR2 comprising the amino acid sequence of SEQ ID NO:12;
iii) H-CDR3 comprising the amino acid sequence of SEQ ID NO:13;
iv) L-CDR1 comprising the amino acid sequence of SEQ ID NO:14;
v) L-CDR2 comprising the amino acid sequence of SEQ ID NO:15; and
vi) L-CDR3 comprising the amino acid sequence of SEQ ID NO:16.

7. The antibody of claim 6, comprising:
i) a VH comprising the amino acid sequence SEQ ID NO: 17; and
ii) a V_{L} comprising the amino acid sequence of SEQ ID NO: 18.

8. The antibody of any one of claims 1-4, wherein the antibody comprises:
i) H-CDR1 comprising the amino acid sequence of SEQ ID NO:19;
ii) H-CDR2 comprising the amino acid sequence of SEQ ID NO:20;
iii) H-CDR3 comprising the amino acid sequence of SEQ ID NO:21;
iv) L-CDR1 comprising the amino acid sequence of SEQ ID NO:22;
v) L-CDR2 comprising the amino acid sequence of SEQ ID NO:23; and
vi) L-CDR3 comprising the amino acid sequence of SEQ ID NO:24.

9. The antibody of claim 8, comprising:
i) a VH comprising the amino acid sequence SEQ ID NO:25; and
ii) a VL comprising the amino acid sequence of SEQ ID NO:26.

10. The antibody of any one of claims 1-4, wherein the antibody comprises:
i) H-CDR1 comprising the amino acid sequence of SEQ ID NO:27;
ii) H-CDR2 comprising the amino acid sequence of SEQ ID NO:28;
iii) H-CDR3 comprising the amino acid sequence of SEQ ID NO:29;
iv) L-CDR1 comprising the amino acid sequence of SEQ ID NO:30;
v) L-CDR2 comprising the amino acid sequence of SEQ ID NO:31; and
vi) L-CDR3 comprising the amino acid sequence of SEQ ID NO:32.

11. The antibody of claim 10, comprising:
i) a VH comprising the amino acid sequence SEQ ID NO:33; and
ii) a VL comprising the amino acid sequence of SEQ ID NO:34.

12. The antibody of any one of claims 1-11, wherein the antibody is selected from the group consisting of: a full length antibody, Fab, Fab′, F(ab)2, F(ab′)2, and scFv.

13. The antibody of any one of claims 1-12, wherein the antibody further comprises an Fc region.

14. The antibody of claim 13, wherein the Fc region comprises an IgG4 sequence.

15. The antibody of claim 14, wherein the Fc region comprises the amino acid sequence of SEQ ID NO:43 or a variant thereof.

16. The antibody of claim 15, wherein the Fc region comprises one or more mutations selected from the group consisting of S228P, M428L and N434A, wherein the mutations are relative to SEQ ID NO:43 under the EU numbering system.

17. The antibody of claim 16, wherein the Fc region comprises mutations S228P, M428L and N434A.

18. The antibody of claim 17, wherein the Fc region comprises amino acid sequence of SEQ ID NO:44.

19. The antibody of any one of claims 1-18, wherein the low-pH dissociation factor of the antibody dissociating from C5 is between about 40% to about 70%.

20. The antibody of any one of claims 1-19, wherein the neutral-pH dissociation factor of the antibody dissociating from C5 is between about 0% to about 10%.

21. The antibody of any one of claims 1-20, wherein the ratio of low-pH dissociation to neutral-pH dissociation is 6 or more.

22. The antibody of any one of claims 1-21, wherein the antibody inhibits binding between human C5a to C5aR.

23. The antibody of any one of claims 1-22, wherein the antibody has a serum half-life in humans that is at least about 25 days.

24. The antibody of any one of claims 1-23, wherein the antibody is manufactured in CHO cells.

25. A nucleic acid encoding the antibody of any one of claims 1-24, comprising the sequence of any one of SEQ ID Nos: 46-53.

26. A vector comprising the nucleic acid of claim 25.

27. A host cell comprising the vector of claim 26.

28. A method for producing the antibody of any one of claims 1-27 under a condition sufficient to allow expression of the antibody by cell.

29. A pharmaceutical composition comprising the antibody of any one of claims 1-24 and a pharmaceutically acceptable carrier.

30. A method for treating an individual having a complement-associated disease or condition, comprising administering to the individual an effective amount of the pharmaceutical composition of claim 29.

31. The method of claim 30, wherein the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD), bullous pemphigoid, hidradenitis suppurativa, dermatitis herpetiformis, sweets syndrome, pyoderma gangrenosum, palmo-plantar pustulosis & pustular psoriasis, rheumatoid neutrophilic dermatoses, subcorneal pustular dermatosis, bowel-associated dermatosis-arthritis syndrome, neutrophilic eccrine hidradenitis, linear IgA disease, or any combinations thereof.

32. A method for reducing the activity of a complement system in an individual, comprising administering to the individual an effective amount of the pharmaceutical composition of claim 29.

33. The antibody of any one of claims 1-24, wherein the antibody cross-reacts with a cyno-monkey C5a or C5.


SEQUENCE LISTING (85 sequences)

SEQ ID NO: 1 - protein, 118 aa, synthetic construct
  REGION 1..118: note = Synthetic construct
QVQLQQSDAELVKPGASVKISCKVSGYTFTDHIIHWMNQRPEQGLEWIGYIYPRDGNTNYNENFKGKATLTADKSSSTAYMQLNSLTSEDSAVYFCARERNLEYFDYWGQGTTLTVSS

SEQ ID NO: 2 - protein, 111 aa, synthetic construct
  REGION 1..111: note = Synthetic construct
DIVLTQSPASLAVSLGQRATISCKASQSVDYDGDNYMNWYQQKPGQPPKLLIYAASNLDSGIPARFSGSGSGTDFTLNIHPVEEEDAATYYCQQSNEDPYTFGGGTKLEIK

SEQ ID NO: 3 - protein, 10 aa, synthetic construct
  REGION 1..10: note = Synthetic construct
GYTFTDHIIH

SEQ ID NO: 4 - protein, 17 aa, synthetic construct
  REGION 1..17: note = Synthetic construct
YIYPREGWTNYNENFKG

SEQ ID NO: 5 - protein, 11 aa, synthetic construct
  REGION 1..11: note = Synthetic construct
ARERNLEYFDY

SEQ ID NO: 6 - protein, 15 aa, synthetic construct
  REGION 1..15: note = Synthetic construct
KASQSVDYEGFNYMN

SEQ ID NO: 7 - protein, 7 aa, synthetic construct
  REGION 1..7: note = Synthetic construct
AASNLDS

SEQ ID NO: 8 - protein, 9 aa, synthetic construct
  REGION 1..9: note = Synthetic construct
QQSNEDPYT

SEQ ID NO: 9 - protein, 118 aa, synthetic construct
  REGION 1..118: note = Synthetic construct
QVQLVQSGAEVKKPGSSVKVSCKASGYTFTDHIIHWMRQAPGQGLEWMGYIYPREGWTNYNENFKGRVTITADKSTSTAYMELSSLRSEDTAVYYCARERNLEYFDYWGQGTTVTVSS

SEQ ID NO: 10 - protein, 111 aa, synthetic construct
  REGION 1..111: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSNEDPYTFGGGTKVEIK

SEQ ID NO: 11 - protein, 10 aa, synthetic construct
  REGION 1..10: note = Synthetic construct
GYTHTDHIIH

SEQ ID NO: 12 - protein, 17 aa, synthetic construct
  REGION 1..17: note = Synthetic construct
YIYPRHGWTNYNENFKG

SEQ ID NO: 13 - protein, 11 aa, synthetic construct
  REGION 1..11: note = Synthetic construct
ARERNLEYFDY

SEQ ID NO: 14 - protein, 15 aa, synthetic construct
  REGION 1..15: note = Synthetic construct
KASQSVDYEGFNYMN

SEQ ID NO: 15 - protein, 7 aa, synthetic construct
  REGION 1..7: note = Synthetic construct
AASNLDS

SEQ ID NO: 16 - protein, 9 aa, synthetic construct
  REGION 1..9: note = Synthetic construct
QQSNEDPHT

SEQ ID NO: 17 - protein, 118 aa, synthetic construct
  REGION 1..118: note = Synthetic construct
QVQLVQSGAEVKKPGSSVKVSCKASGYTHTDHIIHWMRQAPGQGLEWMGYIYPRHGWTNYNENFKGRVTITADKSTSTAYMELSSLRSEDTAVYYCARERNLEYFDYWGQGTTVTVSS

SEQ ID NO: 18 - protein, 111 aa, synthetic construct
  REGION 1..111: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSNEDPHTFGGGTKVEIK

SEQ ID NO: 19 - protein, 10 aa, synthetic construct
  REGION 1..10: note = Synthetic construct
GYTHTDHIIH

SEQ ID NO: 20 - protein, 17 aa, synthetic construct
  REGION 1..17: note = Synthetic construct
YIYPREGWTNYNENFKG

SEQ ID NO: 21 - protein, 11 aa, synthetic construct
  REGION 1..11: note = Synthetic construct
ARERNLEYFDY

SEQ ID NO: 22 - protein, 15 aa, synthetic construct
  REGION 1..15: note = Synthetic construct
KASQSVDYEGFNYMN

SEQ ID NO: 23 - protein, 7 aa, synthetic construct
  REGION 1..7: note = Synthetic construct
AASNLDS

SEQ ID NO: 24 - protein, 9 aa, synthetic construct
  REGION 1..9: note = Synthetic construct
QQSHEDPHT

SEQ ID NO: 25 - protein, 118 aa, synthetic construct
  REGION 1..118: note = Synthetic construct
QVQLVQSGAEVKKPGSSVKVSCKASGYTHTDHIIHWMRQAPGQGLEWMGYIYPREGWTNYNENFKGRVTITADKSTSTAYMELSSLRSEDTAVYYCARERNLEYFDYWGQGTTVTVSS

SEQ ID NO: 26 - protein, 111 aa, synthetic construct
  REGION 1..111: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSHEDPHTFGGGTKVEIK

SEQ ID NO: 27 - protein, 10 aa, synthetic construct
  REGION 1..10: note = Synthetic construct
GYTHTDHIIH

SEQ ID NO: 28 - protein, 17 aa, synthetic construct
  REGION 1..17: note = Synthetic construct
YIYPREGWTNYNENFKG

SEQ ID NO: 29 - protein, 11 aa, synthetic construct
  REGION 1..11: note = Synthetic construct
ARERHLEYFDY

SEQ ID NO: 30 - protein, 15 aa, synthetic construct
  REGION 1..15: note = Synthetic construct
KASQSVDYEGFNYMN

SEQ ID NO: 31 - protein, 7 aa, synthetic construct
  REGION 1..7: note = Synthetic construct
AASNLDS

SEQ ID NO: 32 - protein, 9 aa, synthetic construct
  REGION 1..9: note = Synthetic construct
QQSNEDPHT

SEQ ID NO: 33 - protein, 118 aa, synthetic construct
  REGION 1..118: note = Synthetic construct
QVQLVQSGAEVKKPGSSVKVSCKASGYTHTDHIIHWMRQAPGQGLEWMGYIYPREGWTNYNENFKGRVTITADKSTSTAYMELSSLRSEDTAVYYCARERHLEYFDYWGQGTTVTVSS

SEQ ID NO: 34 - protein, 111 aa, synthetic construct; identical to SEQ ID NO: 18
  REGION 1..111: note = Synthetic construct

SEQ ID NO: 35 - protein, 445 aa, synthetic construct
  REGION 1..445: note = Synthetic construct
QVQLVQSGAEVKKPGSSVKVSCKASGYTFTDHIIHWMRQAPGQGLEWMGYIYPREGWTNYNENFKGRVTITADKSTSTAYMELSSLRSEDTAVYYCARERNLEYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 36 - protein, 218 aa, synthetic construct
  REGION 1..218: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 445 aa, synthetic construct
  REGION 1..445: note = Synthetic construct
QVQLVQSGAEVKKPGSSVKVSCKASGYTHTDHIIHWMRQAPGQGLEWMGYIYPRHGWTNYNENFKGRVTITADKSTSTAYMELSSLRSEDTAVYYCARERNLEYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 38 - protein, 218 aa, synthetic construct
  REGION 1..218: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSNEDPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 445 aa, synthetic construct
  REGION 1..445: note = Synthetic construct
QVQLVQSGAEVKKPGSSVKVSCKASGYTHTDHIIHWMRQAPGQGLEWMGYIYPREGWTNYNENFKGRVTITADKSTSTAYMELSSLRSEDTAVYYCARERNLEYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 40 - protein, 218 aa, synthetic construct
  REGION 1..218: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSHEDPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 445 aa, synthetic construct
  REGION 1..445: note = Synthetic construct
QVQLVQSGAEVKKPGSSVKVSCKASGYTHTDHIIHWMRQAPGQGLEWMGYIYPREGWTNYNENFKGRVTITADKSTSTAYMELSSLRSEDTAVYYCARERHLEYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 42 - protein, 218 aa, synthetic construct; identical to SEQ ID NO: 38
  REGION 1..218: note = Synthetic construct

SEQ ID NO: 43 - protein, 327 aa, synthetic construct
  REGION 1..327: note = Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 327 aa, synthetic construct
  REGION 1..327: note = Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 45 - protein, 74 aa, synthetic construct
  REGION 1..74: note = Synthetic construct
TLQKKIEEIAAKYKHSVVKKCCYDGACVNNDETCEQRAARISLGPRCIKAFTECCVVASQLRANISHKDMQLGR

SEQ ID NO: 46 - DNA, 1335 bp, synthetic construct
  misc_feature 1..1335: note = Synthetic construct
  source 1..1335: mol_type = other DNA
caagtgcagctggtgcagtccggagctgaagtgaagaagcccggcagcagcgtgaaggtgagctgtaaggcctccggctacacattcactgaccacatcatccactggatgaggcaagcccccggccaaggactggagtggatgggctacatctacccaagggagggatggactaactacaacgagaacttcaagggaagggtgacaatcacagccgacaagtccacaagcacagcctacatggaactcagctctctgagaagcgaggatactgccgtgtactactgcgctagggagaggaatctggagtacttcgactactggggccaaggcacaacagtgactgtgagcagcgctagcaccaagggacctagcgtgtttcctctggccccttgtagcagaagcaccagcgaaagcacagccgctctgggctgtctggtgaaagactactttcccgagcccgtgaccgtgtcttggaacagcggagccctgaccagcggagtgcacacatttccagccgtgctccagagcagcggactgtatagcctgagcagcgtggtgaccgtgccttcttctagcctgggcaccaagacctacacctgcaacgtggaccacaagcccagcaacaccaaggtggacaagcgggtggagagcaaatacggccctccttgccctccttgcccagctccagagtttctgggaggacctagcgtgttcctgttccctcccaagcccaaggacaccctgatgatcagccggaccccagaagtcacctgcgtggtggtggacgtgtctcaggaagaccccgaggtgcagttcaactggtacgtggacggcgtggaggtgcacaacgctaagaccaagcccagggaggagcagttcaacagcacctacagggtggtgtccgtgctgacagtgctgcatcaggattggctgaacggcaaggagtacaagtgcaaggtgtccaacaagggcctgcctagcagcatcgagaagaccatcagcaaggccaagggccagcctagagagcctcaggtgtacacactgcccccttctcaggaggagatgaccaagaaccaggtgtccctgacttgcctcgtgaagggcttctaccccagcgatattgccgtggagtgggagtctaacggccagcccgagaacaactacaagaccacccctcccgtgctggatagcgacggctctttcttcctgtacagccggctgacagtggacaaaagtcgctggcaggagggcaacgtgttcagttgcagcgtgctgcacgaggctctgcacgcccactatacccagaagagcctgagcctgagcctgggaaag

SEQ ID NO: 47 - DNA, 654 bp, synthetic construct
  misc_feature 1..654: note = Synthetic construct
  source 1..654: mol_type = other DNA
gatattgtgctgacacagtctccagcttctctggcagtgtctccaggacagagagctacaattacttgtaaggcctctcagtccgtggattacgagggcttcaactacatgaactggtatcagcagaaaccaggacagcctcctaaactgctgatctacgccgcttctaatctggattccggagtgccagcaagattttccggctccggctctggcaccgattttaccctgaccatcaatccagtggaagccgaggataccgctaactactattgccagcagtctaacgaggacccttatacatttggcggcggaacaaaggtggagattaagcgtacggtggccgctcctagcgtgttcatcttccctcccagcgacgagcagctgaaaagcggaacagccagcgtcgtctgcctgctgaataacttctaccccagagaggccaaagtccagtggaaagtggacaacgccctccagagcggaaactctcaggagagcgtgaccgagcaggacagcaaggacagcacctacagcctgagcagcacactgaccctgagcaaggccgactacgagaagcacaaggtgtacgcttgcgaggtcacacaccagggactgtctagcccagtgaccaagagcttcaaccgcggcgagtgt

SEQ ID NO: 48 - DNA, 1335 bp, synthetic construct
  misc_feature 1..1335: note = Synthetic construct
  source 1..1335: mol_type = other DNA
caagtgcagctggtgcagtccggagctgaagtgaagaagcccggcagcagcgtgaaggtgagctgtaaggcctccggctacacacacactgaccacatcatccactggatgaggcaagcccccggccaaggactggagtggatgggctacatctacccaaggcacggatggactaactacaacgagaacttcaagggaagggtgacaatcacagccgacaagtccacaagcacagcctacatggaactcagctctctgagaagcgaggatactgccgtgtactactgcgctagggagaggaatctggagtacttcgactactggggccaaggcacaacagtgactgtgagcagcgctagcaccaagggacctagcgtgtttcctctggccccttgtagcagaagcaccagcgaaagcacagccgctctgggctgtctggtgaaagactactttcccgagcccgtgaccgtgtcttggaacagcggagccctgaccagcggagtgcacacatttccagccgtgctccagagcagcggactgtatagcctgagcagcgtggtgaccgtgccttcttctagcctgggcaccaagacctacacctgcaacgtggaccacaagcccagcaacaccaaggtggacaagcgggtggagagcaaatacggccctccttgccctccttgcccagctccagagtttctgggaggacctagcgtgttcctgttccctcccaagcccaaggacaccctgatgatcagccggaccccagaagtcacctgcgtggtggtggacgtgtctcaggaagaccccgaggtgcagttcaactggtacgtggacggcgtggaggtgcacaacgctaagaccaagcccagggaggagcagttcaacagcacctacagggtggtgtccgtgctgacagtgctgcatcaggattggctgaacggcaaggagtacaagtgcaaggtgtccaacaagggcctgcctagcagcatcgagaagaccatcagcaaggccaagggccagcctagagagcctcaggtgtacacactgcccccttctcaggaggagatgaccaagaaccaggtgtccctgacttgcctcgtgaagggcttctaccccagcgatattgccgtggagtgggagtctaacggccagcccgagaacaactacaagaccacccctcccgtgctggatagcgacggctctttcttcctgtacagccggctgacagtggacaaaagtcgctggcaggagggcaacgtgttcagttgcagcgtgctgcacgaggctctgcacgcccactatacccagaagagcctgagcctgagcctgggaaag

SEQ ID NO: 49 - DNA, 654 bp, synthetic construct
  misc_feature 1..654: note = Synthetic construct
  source 1..654: mol_type = other DNA
gatattgtgctgacacagtctccagcttctctggcagtgtctccaggacagagagctacaattacttgtaaggcctctcagtccgtggattacgagggcttcaactacatgaactggtatcagcagaaaccaggacagcctcctaaactgctgatctacgccgcttctaatctggattccggagtgccagcaagattttccggctccggctctggcaccgattttaccctgaccatcaatccagtggaagccgaggataccgctaactactattgccagcagtctaacgaggaccctcacacatttggcggcggaacaaaggtggagattaagcgtacggtggccgctcctagcgtgttcatcttccctcccagcgacgagcagctgaaaagcggaacagccagcgtcgtctgcctgctgaataacttctaccccagagaggccaaagtccagtggaaagtggacaacgccctccagagcggaaactctcaggagagcgtgaccgagcaggacagcaaggacagcacctacagcctgagcagcacactgaccctgagcaaggccgactacgagaagcacaaggtgtacgcttgcgaggtcacacaccagggactgtctagcccagtgaccaagagcttcaaccgcggcgagtgt

SEQ ID NO: 50 - DNA, 1335 bp, synthetic construct
  misc_feature 1..1335: note = Synthetic construct
  source 1..1335: mol_type = other DNA
caagtgcagctggtgcagtccggagctgaagtgaagaagcccggcagcagcgtgaaggtgagctgtaaggcctccggctacacacacactgaccacatcatccactggatgaggcaagcccccggccaaggactggagtggatgggctacatctacccaagggagggatggactaactacaacgagaacttcaagggaagggtgacaatcacagccgacaagtccacaagcacagcctacatggaactcagctctctgagaagcgaggatactgccgtgtactactgcgctagggagaggcacctggagtacttcgactactggggccaaggcacaacagtgactgtgagcagcgctagcaccaagggacctagcgtgtttcctctggccccttgtagcagaagcaccagcgaaagcacagccgctctgggctgtctggtgaaagactactttcccgagcccgtgaccgtgtcttggaacagcggagccctgaccagcggagtgcacacatttccagccgtgctccagagcagcggactgtatagcctgagcagcgtggtgaccgtgccttcttctagcctgggcaccaagacctacacctgcaacgtggaccacaagcccagcaacaccaaggtggacaagcgggtggagagcaaatacggccctccttgccctccttgcccagctccagagtttctgggaggacctagcgtgttcctgttccctcccaagcccaaggacaccctgatgatcagccggaccccagaagtcacctgcgtggtggtggacgtgtctcaggaagaccccgaggtgcagttcaactggtacgtggacggcgtggaggtgcacaacgctaagaccaagcccagggaggagcagttcaacagcacctacagggtggtgtccgtgctgacagtgctgcatcaggattggctgaacggcaaggagtacaagtgcaaggtgtccaacaagggcctgcctagcagcatcgagaagaccatcagcaaggccaagggccagcctagagagcctcaggtgtacacactgcccccttctcaggaggagatgaccaagaaccaggtgtccctgacttgcctcgtgaagggcttctaccccagcgatattgccgtggagtgggagtctaacggccagcccgagaacaactacaagaccacccctcccgtgctggatagcgacggctctttcttcctgtacagccggctgacagtggacaaaagtcgctggcaggagggcaacgtgttcagttgcagcgtgctgcacgaggctctgcacgcccactatacccagaagagcctgagcctgagcctgggaaag

SEQ ID NO: 51 - DNA, 654 bp, synthetic construct; identical to SEQ ID NO: 49
  misc_feature 1..654: note = Synthetic construct
  source 1..654: mol_type = other DNA

SEQ ID NO: 52 - DNA, 1335 bp, synthetic construct
  misc_feature 1..1335: note = Synthetic construct
  source 1..1335: mol_type = other DNA
caagtgcagctggtgcagtccggagctgaagtgaagaagcccggcagcagcgtgaaggtgagctgtaaggcctccggctacacacacactgaccacatcatccactggatgaggcaagcccccggccaaggactggagtggatgggctacatctacccaagggagggatggactaactacaacgagaacttcaagggaagggtgacaatcacagccgacaagtccacaagcacagcctacatggaactcagctctctgagaagcgaggatactgccgtgtactactgcgctagggagaggaatctggagtacttcgactactggggccaaggcacaacagtgactgtgagcagcgctagcaccaagggacctagcgtgtttcctctggccccttgtagcagaagcaccagcgaaagcacagccgctctgggctgtctggtgaaagactactttcccgagcccgtgaccgtgtcttggaacagcggagccctgaccagcggagtgcacacatttccagccgtgctccagagcagcggactgtatagcctgagcagcgtggtgaccgtgccttcttctagcctgggcaccaagacctacacctgcaacgtggaccacaagcccagcaacaccaaggtggacaagcgggtggagagcaaatacggccctccttgccctccttgcccagctccagagtttctgggaggacctagcgtgttcctgttccctcccaagcccaaggacaccctgatgatcagccggaccccagaagtcacctgcgtggtggtggacgtgtctcaggaagaccccgaggtgcagttcaactggtacgtggacggcgtggaggtgcacaacgctaagaccaagcccagggaggagcagttcaacagcacctacagggtggtgtccgtgctgacagtgctgcatcaggattggctgaacggcaaggagtacaagtgcaaggtgtccaacaagggcctgcctagcagcatcgagaagaccatcagcaaggccaagggccagcctagagagcctcaggtgtacacactgcccccttctcaggaggagatgaccaagaaccaggtgtccctgacttgcctcgtgaagggcttctaccccagcgatattgccgtggagtgggagtctaacggccagcccgagaacaactacaagaccacccctcccgtgctggatagcgacggctctttcttcctgtacagccggctgacagtggacaaaagtcgctggcaggagggcaacgtgttcagttgcagcgtgctgcacgaggctctgcacgcccactatacccagaagagcctgagcctgagcctgggaaag

SEQ ID NO: 53 - DNA, 654 bp, synthetic construct
  misc_feature 1..654: note = Synthetic construct
  source 1..654: mol_type = other DNA
gatattgtgctgacacagtctccagcttctctggcagtgtctccaggacagagagctacaattacttgtaaggcctctcagtccgtggattacgagggcttcaactacatgaactggtatcagcagaaaccaggacagcctcctaaactgctgatctacgccgcttctaatctggattccggagtgccagcaagattttccggctccggctctggcaccgattttaccctgaccatcaatccagtggaagccgaggataccgctaactactattgccagcagtctcacgaggaccctcacacatttggcggcggaacaaaggtggagattaagcgtacggtggccgctcctagcgtgttcatcttccctcccagcgacgagcagctgaaaagcggaacagccagcgtcgtctgcctgctgaataacttctaccccagagaggccaaagtccagtggaaagtggacaacgccctccagagcggaaactctcaggagagcgtgaccgagcaggacagcaaggacagcacctacagcctgagcagcacactgaccctgagcaaggccgactacgagaagcacaaggtgtacgcttgcgaggtcacacaccagggactgtctagcccagtgaccaagagcttcaaccgcggcgagtgt

SEQ ID NO: 54 - protein, 118 aa, synthetic construct; identical to SEQ ID NO: 9
  REGION 1..118: note = Synthetic construct

SEQ ID NO: 55 - protein, 111 aa, synthetic construct
  REGION 1..111: note = Synthetic construct
DIVLTQSPASLAVSPGQRATITCKASQSVDYEGDNYMNWYQQKPGQPPKLLIYAASNLDSGVPARFSGSGSGTDFTLTINPVEAEDTANYYCQQSNEDPHTFGGGTKVEIK

SEQ ID NO: 56 - protein, 118 aa, synthetic construct; identical to SEQ ID NO: 9
  REGION 1..118: note = Synthetic construct

SEQ ID NO: 57 - protein, 111 aa, synthetic construct
  REGION 1..111: note = Synthetic construct
DIVLTQSPASLAVSPGQRATITCKASQSVDFEGFNYMNWYQQKPGQPPKLLIYAASNLDSGVPARFSGSGSGTDFTLTINPVEAEDTANYYCQQSVEDPHTFGGGTKVEIK

SEQ ID NO: 58 - protein, 118 aa, synthetic construct; identical to SEQ ID NO: 9
  REGION 1..118: note = Synthetic construct

SEQ ID NO: 59 - protein, 111 aa, synthetic construct
  REGION 1..111: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSVEDPHTFGGGTKVEIK

SEQ ID NO: 60 - protein, 118 aa, synthetic construct; identical to SEQ ID NO: 9
  REGION 1..118: note = Synthetic construct

SEQ ID NO: 61 - protein, 111 aa, synthetic construct
  REGION 1..111: note = Synthetic construct
DIVLTQSPASLAVSPGQRATITCKASQSVDYEGFNYMNWYQQKPGQPPKLLIYAASNLDSGVPARFSGSGSGTDFTLTINPVEAEDTANYYCQQSLEDPHTFGGGTKVEIK

SEQ ID NO: 62 - protein, 118 aa, synthetic construct
  REGION 1..118: note = Synthetic construct
QVQLVQSGAEVKKPGSSVKVSCKASGYTFTDHIIHWMRQAPGQGLEWMGYIYPREGFTNYNENFKGRVTITADKSTSTAYMELSSLRSEDTAVYYCARERNLEYFDYWGQGTTVTVSS

SEQ ID NO: 63 - protein, 111 aa, synthetic construct; identical to SEQ ID NO: 57
  REGION 1..111: note = Synthetic construct

SEQ ID NO: 64 - protein, 118 aa, synthetic construct; identical to SEQ ID NO: 9
  REGION 1..118: note = Synthetic construct

SEQ ID NO: 65 - protein, 111 aa, synthetic construct
  REGION 1..111: note = Synthetic construct
DIVLTQSPASLAVSPGQRATITCKASQSVDFEGFNYMNWYQQKPGQPPKLLIYAASNLDSGVPARFSGSGSGTDFTLTINPVEAEDTANYYCQQSNEDPHTFGGGTKVEIK

SEQ ID NO: 66 - protein, 118 aa, synthetic construct; identical to SEQ ID NO: 9
  REGION 1..118: note = Synthetic construct

SEQ ID NO: 67 - protein, 111 aa, synthetic construct
  REGION 1..111: note = Synthetic construct
DIVLTQSPASLAVSPGQRATITCKASQSVDFEGFNYMNWYQQKPGQPPKLLIYAASNLDSGVPARFSGSGSGTDFTLTINPVEAEDTANYYCQQSLEDPHTFGGGTKVEIK

SEQ ID NO: 68 - protein, 118 aa, synthetic construct; identical to SEQ ID NO: 9
  REGION 1..118: note = Synthetic construct

SEQ ID NO: 69 - protein, 111 aa, synthetic construct
  REGION 1..111: note = Synthetic construct
DIVLTQSPASLAVSPGQRATITCKASQSVDFEGDNYMNWYQQKPGQPPKLLIYATSNLDSGVPARFSGSGSGTDFTLTINPVEAEDTANYYCQQSVEDPHTFGGGTKVEIK

SEQ ID NO: 70 - protein, 118 aa, synthetic construct; identical to SEQ ID NO: 9
  REGION 1..118: note = Synthetic construct

SEQ ID NO: 71 - protein, 111 aa, synthetic construct
  REGION 1..111: note = Synthetic construct
DIVLTQSPASLAVSPGQRATITCKASQSVDYEGDNYMNWYQQKPGQPPKLLIYATSNLDSGVPARFSGSGSGTDFTLTINPVEAEDTANYYCQQSLEDPHTFGGGTKVEIK

SEQ ID NO: 72 - protein, 118 aa, synthetic construct; identical to SEQ ID NO: 9
  REGION 1..118: note = Synthetic construct

SEQ ID NO: 73 - protein, 111 aa, synthetic construct
  REGION 1..111: note = Synthetic construct
DIVLTQSPASLAVSPGQRATITCKASQSVDYEGFNYMNWYQQKPGQPPKLLIYATSNLDSGVPARFSGSGSGTDFTLTINPVEAEDTANYYCQQSVEDPHTFGGGTKVEIK

SEQ ID NO: 74 - protein, 118 aa, synthetic construct; identical to SEQ ID NO: 9
  REGION 1..118: note = Synthetic construct

SEQ ID NO: 75 - protein, 111 aa, synthetic construct
  REGION 1..111: note = Synthetic construct
DIVLTQSPASLAVSPGQRATITCKASQSVDFEGDNYMNWYQQKPGQPPKLLIYATSNLDSGVPARFSGSGSGTDFTLTINPVEAEDTANYYCQQSLEDPHTFGGGTKVEIK

SEQ ID NO: 76 - protein, 118 aa, synthetic construct; identical to SEQ ID NO: 9
  REGION 1..118: note = Synthetic construct

SEQ ID NO: 77 - protein, 111 aa, synthetic construct
  REGION 1..111: note = Synthetic construct
DIVLTQSPASLAVSPGQRATITCKASQSVDYEGFNYMNWYQQKPGQPPKLLIYATSNLDSGVPARFSGSGSGTDFTLTINPVEAEDTANYYCQQSLEDPHTFGGGTKVEIK

SEQ ID NO: 78 - protein, 118 aa, synthetic construct; identical to SEQ ID NO: 9
  REGION 1..118: note = Synthetic construct

SEQ ID NO: 79 - protein, 111 aa, synthetic construct
  REGION 1..111: note = Synthetic construct
DIVLTQSPASLAVSPGQRATITCKASQSVDFEGFNYMNWYQQKPGQPPKLLIYATSNLDSGVPARFSGSGSGTDFTLTINPVEAEDTANYYCQQSVEDPHTFGGGTKVEIK

SEQ ID NO: 80 - protein, 118 aa, synthetic construct; identical to SEQ ID NO: 9
  REGION 1..118: note = Synthetic construct

SEQ ID NO: 81 - protein, 111 aa, synthetic construct
  REGION 1..111: note = Synthetic construct
DIVLTQSPASLAVSPGQRATITCKASQSVDYEGDNYMNWYQQKPGQPPKLLIYATSNLDSGVPARFSGSGSGTDFTLTINPVEAEDTANYYCQQSVEDPHTFGGGTKVEIK

SEQ ID NO: 82 - protein, 118 aa, synthetic construct
  REGION 1..118: note = Synthetic construct
QVQLVQSGAEVKKPGSSVKVSCKASGYTFTDHIIHWMRQAPGQGLEWMGYIYPREGNTNYNENFKGRVTITADKSTSTAYMELSSLRSEDTAVYYCARERNLEYFDYWGQGTTVTVSS

SEQ ID NO: 83 - protein, 111 aa, synthetic construct; identical to SEQ ID NO: 55
  REGION 1..111: note = Synthetic construct

SEQ ID NO: 84 - protein, 118 aa, synthetic construct
  REGION 1..118: note = Synthetic construct
QVQLVQSGAEVKKPGSSVKVSCKASGYTFTDHIIHWMRQAPGQGLEWMGYIYPRDGNTNYNENFKGRVTITADKSTSTAYMELSSLRSEDTAVYYCARERNLEYFDYWGQGTTVTVSS

SEQ ID NO: 85 - protein, 111 aa, synthetic construct
  REGION 1..111: note = Synthetic construct
DIVLTQSPASLAVSPGQRATITCKASQSVDYDGDNYMNWYQQKPGQPPKLLIYAASNLDSGVPARFSGSGSGTDFTLTINPVEAEDTANYYCQQSNEDPYTFGGGTKVEIK